(19) United States
(12) Patent Application Publication
(10) Pub. No.: US 2013/0071881 A1
(43) Pub. Date: Mar. 21, 2013

(54) METHODS FOR PRODUCING ANTIBODY-PRODUCING CELLS THAT PRODUCE DESIRED POLYPEPTIDES

(71) Applicants: Hitoshi Ohmori, Okayama (JP); Naoki Kanayama, Okayama (JP); Shinobu Fujii, Hiroshima (JP)

(72) Inventors: Hitoshi Ohmori, Okayama (JP); Naoki Kanayama, Okayama (JP); Shinobu Fujii, Hiroshima (JP)

(73) Assignee: Immuno Tec Laboratory Co. Ltd, Okayama-shi Okayama (JP)

(21) Appl. No.: 13/510,910

(22) PCT Filed: Nov. 18, 2010
(86) PCT No.: PCT/JP2010/006759

§ 371 Date: Aug. 6, 2012

Related U.S. Application Data
(60) Provisional application No. 61/263,285, filed on Nov. 20, 2009.

(30) Foreign Application Priority Data
Nov. 19, 2009 (JP) 2009-264398

(51) Int. Cl. C12N 15/66

(52) U.S. Cl. CPC C12N 15/66
USPC 435/91.41; 435/326; 435/455; 435/320.1

(57) ABSTRACT

An objective of the present invention is to provide methods for introducing DNAs encoding a desired amino acid sequence into a region comprising a DNA encoding an antibody variable region of antibody-producing cells. The present inventors developed methods for efficiently introducing DNAs encoding a desired amino acid sequence into the antibody variable region gene locus of DT40-SW, which is a mutant line of the DT40 chicken B cell line which has the ability to spontaneously introduce mutations. This allows mutagenesis of introduced DNAs to modify the polypeptides to have superior functions. In particular, the present inventors revealed the nucleotide sequence of the antibody H chain variable region gene locus of the DT40 cell line. Based on this finding, the present inventors successfully constructed targeting vectors that allow efficient substitution of the antibody H chain variable region gene locus of the DT40 cell line with a gene encoding a desired polypeptide.

### TECHNICAL FIELD

The present application claims the benefit of Japanese Patent Application No. 2009-264398 filed on Nov. 19, 2009, and U.S. Provisional Patent Application No. 61/263,285, filed on Nov. 20, 2009, the entire contents of which are incorporated by reference herein.

The present invention relates to methods for introducing a DNA encoding a desired polypeptide into a region comprising a DNA encoding an antibody variable region of antibody-producing cells. The present invention also relates to methods for producing antibody-producing cells that produce polypeptides into which mutations are introduced, and methods for producing polypeptides into which mutations are introduced. The present invention further relates to cells and gene targeting vectors used in the above methods.

### BACKGROUND ART

The affinity of antibodies produced in response to antigen stimulation is elevated over time in vivo. This is called “affinity maturation”. In antigen-specific B cells that actively divide upon activation, somatic mutation occurs in antibody variable region genes at high frequency. The affinity maturation proceeds by strict selection of highaffinity B cell clones from the resulting population of various mutant B cells. On the basis of this principle, monoclonal antibodies are routinely produced by repeated immunization to animals and hybridoma preparation. However, antibody preparation requires a lot of effort and time, and the antigen specificity or affinity of the prepared antibodies cannot be altered. Nevertheless, in many cases, the specificity or affinity of the prepared antibodies needs to be further improved for use as pharmaceutical or diagnostic agents. For this purpose, the phage display method, which is an in vitro technique, has been commonly used to date. In the phage display method, the process of antibody selection can be performed more rapidly than in the hybridoma method. However, the phage display method is known to have problems. For example, the success of antibody production largely depends on the quality of library, and since the Fv domain is displayed as a recombinant scFv on phage, the specificity is often altered when the whole antibody is prepared from the scFv and expressed. In particular, preparation of a mutant library, which is key to this process, requires huge effort and advanced DNA recombinant technology.

It is thought that antibodies can be produced rapidly and efficiently if the in vivo antibody production system is reproduced using an in vitro culture cell system. The DT40 chicken B cell line which has the ability to introduce mutations into antibody genes is suitable for this purpose for the following reasons.

(1) A variety of antibody libraries can be produced by simply culturing the DT40 cells due to their ability to spontaneously introduce mutations.

(2) Specific clones can be selected based on antigen binding because the DT40 cells express antibodies on the cell surface and secrete them into the culture supernatant.

(3) The functions of DT40 cells can be readily modified by gene knockout or such due to the very high homologous recombination efficiency.

The present inventors established the DT40-SW cell line, in which a mutation feature of DT40 can be optionally switched on and off. Then, the present inventors developed a method for producing antibodies in vitro using DT40-SW (Patent Document 1 and Non-patent Document 1). Using this method, the present inventors successfully prepared antibodies against various antigens, including antibodies that were difficult to obtain by conventional methods, from antibody libraries obtained by switching on a mutation feature and culturing (Non-patent Documents 2 and 3). Furthermore, this method allows affinity maturation of antibodies by further introducing mutations into the prepared antibody-producing cells to produce various antibodies, and repeating selection of the antibodies (Patent Document 2). The antibodies prepared by this method are chicken IgM antibodies, and affinity maturation is only possible for these antibodies prepared by this method only. Accordingly, if this method can be applied to antibodies prepared by the hybridoma method, the phage display method, or the like, it will become a very useful technique since it can be utilized to improve the specificity and affinity of accumulated monoclonal antibodies.

In vitro systems such as the phage display method need to be used to alter the properties of antibodies obtained by the hybridoma method. However, the phage display-based functional alteration of antibodies is not necessarily a simple technique. Meanwhile, cell lines that have mutational ability, such as DT40, have been used as libraries by introducing mutations into their own antibody genes (Non-patent Documents 2, 4, and 5). However, there is no report on modification of foreign antibody genes using such cell lines.

### CITATION LIST

### Patent Literature

PTL 1: Japanese Patent Application Kokai Publication No. (JP-A) 2006-109711 (unexamined, published Japanese patent application)
PTL 2: JP-A (Kokai) 2009-60850
PTL 3: WO2007/026661

### Non Patent Literature

NPL 1: Kanayama, N., et al. Biochem. Biophys. Res. Commun. 327, 70-75
NPL 2: Todo, K., et al. J. Biosci. Bioeng. 102, 478-481 (2006)
NPL 3: Kanayama, N., et al. YAKUGAKU ZASSHI 129, 11-17 (2009)
NPL 4: Cumbers, S. J., et al. Nat. Biotechnol. 20, 1129-1134 (2002)
NPL 5: Seo, H., et al. Nat. Biotechnol. 23, 731-735 (2005)
NPL 6: Fawell et al. Characterization and colocalization of steroid binding and dimerization activities in the mouse estrogen receptor. Cell (1990) vol. 60 (6) pp. 953-62
NPL 7: Danielian et al. Identification of residues in the estrogen receptor that confer differential sensitivity to estrogen and hydroxytamoxifen. Mol Endocrinol (1993) vol. 7 (2) pp. 232-40
NPL 8: Littlewood et al. A modified oestrogen receptor ligand-binding domain as an improved switch for the regulation of heterologous proteins. Nucleic Acids Res (1995) vol. 23 (10) pp. 1686
NPL 9: Zhang et al. Inducible site-directed recombination in mouse embryonic stem cells. Nucleic Acids Res (1996) vol. 24 (4) pp. 543-8

### SUMMARY OF INVENTION

### Technical Problem

If genes encoding a polypeptide comprising a desired amino acid sequence can be introduced into antibody gene loci of antibody-producing cells such as DT40, the DNAs can be modified using the mutation-introducing ability of the antibody-producing cells. However, an effective technique for introducing foreign DNAs into antibody gene loci of antibody-producing cells has not been developed.

The present invention was achieved in view of the above circumstances. An objective of the present invention is to provide methods for introducing a DNA encoding a desired amino acid sequence into a region comprising a DNA encoding an antibody variable region of antibody-producing cells.

Another objective of the present invention is to provide methods for producing antibody-producing cells that produce polypeptides, methods for producing polypeptides, methods for producing antibody-producing cells that produce polypeptides into which mutations are introduced, and methods for producing polypeptides into which mutations are introduced.

Still another objective of the present invention is to provide cells and kits comprising the cells for use in the above methods.

Yet another objective of the present invention is to provide gene targeting vectors, and cells and kits that comprise the vectors for use in the above methods.

Another objective of the present invention is to provide DNAs comprising a DNA encoding a chicken antibody heavy chain variable region, vectors comprising the DNAs, and cells comprising the DNAs or vectors.

### Solution to Problem

The present inventors developed a method for efficiently introducing a DNA encoding a desired amino acid sequence into the antibody variable region gene locus of DT40-SW, which is a mutant cell line derived from the DT40 chicken B cell line having an autonomous ability to introduce mutations. This method allows modification of polypeptides to have superior functions by mutating the introduced DNAs. In particular, the present inventors revealed the nucleotide sequence of the antibody heavy chain variable region gene locus of the DT40 cell line. By this, the present inventors successfully constructed targeting vectors capable of efficiently replacing the antibody heavy chain variable region gene locus of the DT40 cell line with a DNA encoding a desired amino acid sequence.

The present invention is based on the above findings, and relates to the following:

[1] A method of homologously recombining a DNA construct and a region comprising a DNA encoding an antibody variable region of an antibody-producing cell, which comprises the step of introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct.

[2] The method of [1], wherein the DNA of (3) inhibits the production of a polypeptide comprising the desired amino acid sequence, and is located between two site-specific recombinase recognition sequences oriented in the same direction, and wherein the DNA of (3) comprises a promoter DNA and a marker gene that function in the cell, and the DNA of (3) can be removed from the DNA construct by a site-specific recombinase.

[3] A method for selecting a cell, which comprises the steps of:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct; and

(b) selecting a cell in which homologous recombination has occurred between the DNA construct and the region comprising a DNA encoding an antibody variable region of the antibody-producing cell.

[4] The method of [3], wherein the DNA of (3) inhibits the production of a polypeptide comprising the desired amino acid sequence, and is located between two site-specific recombinase recognition sequences oriented in the same direction, and wherein the DNA of (3) comprises a promoter DNA and a marker gene that function in the cell, and the DNA of (3) can be removed from the DNA construct by a site-specific recombinase.

[5] A method for producing an antibody-producing cell that produces a polypeptide, which comprises the steps of:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct;

(b) selecting a cell in which homologous recombination has occurred between the DNA construct and the region comprising a DNA encoding an antibody variable region of the antibody-producing cell; and

(c) removing the DNA of (3) from the genomic DNA of the cell selected in step (b).

[6] The method of [5], wherein the DNA of (3) inhibits the production of the polypeptide comprising the desired amino acid sequence and is located between two site-specific recombinase recognition sequences oriented in the same direction, and wherein the DNA of (3) comprises a promoter DNA and a marker gene that function in the cell, and the DNA of (3) can be removed from the DNA construct by a site-specific recombinase.

[7] The method of [5], wherein the steps of (a) to (c) are defined as follows:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;

(b) selecting a cell in which homologous recombination has occurred between the DNA construct and the region comprising a DNA encoding an antibody variable region of the antibody-producing cell; and

(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell.

[8] The method of any one of [5] to [7], wherein the produced polypeptide is displayed on the surface of the cell, and/or secreted to the outside of the cell.

[9] The method of any one of [4], [6], and [7], wherein the cell is selected using the expression of the marker gene as an indicator.

[10] The method of any one of [3], [4], [6], [7], and [9], wherein the cell is selected using the absence of endogenous antibody expression in the antibody-producing cell as an indicator.

[11] A method for producing an antibody-producing cell that produces a polypeptide into which a mutation is introduced, which comprises the steps of:

(a) allowing homologous recombination between a DNA construct and a region comprising a DNA encoding an antibody variable region of an antibody-producing cell expressing the AID gene, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct;

(b) selecting a cell in which homologous recombination has occurred between the DNA construct and the region comprising a DNA encoding an antibody variable region of the antibody-producing cell; and

(c) removing the DNA of (3) from the genomic DNA of the cell selected in step (b).

[12] The method of [11], wherein the steps of (a) to (c) are defined as follows:

(a) allowing homologous recombination between a DNA construct and a region comprising a DNA encoding an antibody variable region of an antibody-producing cell expressing the AID gene, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of the polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;

(b) selecting a cell in which homologous recombination has occurred between the DNA construct and the region comprising a DNA encoding an antibody variable region of the antibody-producing cell; and

(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell.

[13] A method for producing an antibody-producing cell that produces a polypeptide into which a mutation is introduced, which comprises the steps of:

(a) allowing homologous recombination between a DNA construct and a region comprising a DNA encoding an antibody variable region of an antibody-producing cell in which AID gene expression can be artificially switched on and off, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct;

(b) selecting a cell in which homologous recombination has occurred between the DNA construct and the region comprising a DNA encoding an antibody variable region of the antibody-producing cell;

(c) removing the DNA of (3) from the genomic DNA of the cell selected in step (b);

(d) switching the AID gene expression on and off; and

(e) selecting a cell expressing the AID gene.

[14] The method of [13], wherein the steps of (a) to (e) are defined as follows:

(a) allowing homologous recombination between a DNA construct and a region comprising a DNA encoding an antibody variable region of an antibody-producing cell, by introducing a targeting vector into the antibody-producing cell, wherein the targeting vector comprises a DNA construct comprising:

(1) a promoter DNA that functions in the cell;

(2) a DNA that encodes a desired amino acid sequence; and

(3) a DNA that inhibits the production of the polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase, wherein the endogenous AID gene in the antibody-producing cell is functionally destroyed, and the antibody-producing cell comprises a DNA construct comprising:

a promoter DNA that functions in the cell,

a DNA that is located between two site-specific recombinase recognition sequences oriented in opposite directions, and which can be inverted by a site-specific recombinase, and comprises an exogenous AID gene;

(b) selecting a cell in which homologous recombination has occurred between the DNA construct and the region comprising a DNA encoding an antibody variable region of the antibody-producing cell;

(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell;

(d) inverting the DNA located between two site-specific recombinase recognition sequences oriented in opposite directions by reacting a site-specific recombinase at the site-specific recombinase recognition sequences in the genome of the cell selected in step (b), to switch the AID gene expression on and off; and

(e) selecting a cell expressing the AID gene.

[15] The method of [13], wherein the steps of (a) to (e) are defined as follows:

(a) allowing homologous recombination between a DNA construct and a region comprising a DNA encoding an antibody variable region of an antibody-producing cell, by introducing a targeting vector into the antibody-producing cell,

wherein the endogenous AID gene in the antibody-producing cell is functionally destroyed and the antibody-producing cell comprises:

a DNA construct comprising

a promoter DNA that functions in the cell, and

a DNA that is located between two site-specific recombinase recognition sequences oriented in the opposite directions, and which can be inverted by a site-specific recombinase, and comprises an exogenous AID gene, and

a DNA construct comprising a promoter DNA that functions in the cell and a DNA encoding the site-specific recombinase,

wherein in the antibody-producing cell, the site-specific recombinase is activated in the presence of an extracellular stimulus and is not activated in the absence of the extracellular stimulus,

wherein the targeting vector comprises a DNA construct comprising:

(1) a promoter DNA that functions in the cell;

(2) a DNA that encodes a desired amino acid sequence; and

(3) a DNA that inhibits the production of a polypeptide comprising the desired amino acid sequence, and is located between two site-specific recombinase recognition sequences oriented in the same orientation, and which comprises a promoter DNA and a marker gene that function in the cell, and can be removed from the DNA construct by the site-specific recombinase;

(b) selecting a cell in which homologous recombination has occurred between the DNA construct and the region comprising a DNA encoding an antibody variable region of the antibody-producing cell;

(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell via activation of the site-specific recombinase activity by an extracellular stimulus to the cell;

(d) inverting the DNA located between the two site-specific recombinase recognition sequences oriented in opposite directions by reacting a site-specific recombinase at the site-specific recombinase recognition sequences in the genome of the cell selected in step (b), via activation of the site-specific recombinase activity by an extracellular stimulus to the cell, to switch the AID gene expression on and off; and

(e) selecting a cell expressing the AID gene.

[16] The method of any one of [11] to [15], wherein the produced polypeptide into which a mutation is introduced is displayed on the surface of the cell and/or secreted to the outside of the cell.

[17] The method of any one of [11] to [16], wherein the cell is selected in step (b) using the expression of the marker gene as an indicator.

[18] The method of any one of [11] to [17], wherein the cell is selected in step (b) using the absence of endogenous antibody expression in the antibody-producing cell as an indicator.

[19] The method of [15], wherein the site-specific recombinase is a fusion protein of a site-specific recombinase with an estrogen receptor or a protein comprising an estrogen-binding domain thereof; and wherein a ligand capable of binding to the estrogen-binding domain serves as the extracellular stimulus.

[20] The method of [19], wherein the estrogen receptor or estrogen-binding domain thereof is a mouse mutant estrogen receptor in which glycine is substituted with arginine at amino acid position 525, or a mouse mutant estrogen-binding domain thereof; and wherein the ligand capable of binding to the estrogen-binding domain is 4-hydroxytamoxifen.

[21] The method of any one of [11] to [20], wherein the antibody-producing cell has the following properties:

(a) only one of the two alleles of the XRCC3 gene is inactivated in the antibody-producing cell; and

(b) the frequency of introduction of a point mutation is elevated as compared to a cell having the two alleles of the XRCC3 gene.

[22] The method of [21], wherein the sixth exon is inactivated in either one of the two alleles of the XRCC3 gene.

[23] The method of any one of [11] to [22], wherein the antibody-producing cell is a B cell.

[24] The method of [23], wherein the B cell is derived from chicken.

[25] The method of any one of [2], [4], [6], [7], [9], [10], [12], [14], and [15], wherein the combination of site-specific recombinase and site-specific recombinase recognition sequence is (i) or (ii) below:

(i) the site-specific recombinase is a Cre recombinase, and the site-specific recombinase recognition sequence is a loxP sequence; and

(ii) the site-specific recombinase is an FLP recombinase, and the site-specific recombinase recognition sequence is an FRT sequence.

[26] The method of any one of [1] to [25], wherein the polypeptide comprising a desired amino acid sequence comprises an amino acid sequence of an antibody constant region and a desired amino acid sequence.

[27] The method of [26], wherein the desired amino acid sequence is an amino acid sequence of an antibody variable region.

[28] The method of any one of [1] to [25], wherein the polypeptide comprising a desired amino acid sequence is an antibody heavy or light chain.

[29] A method for producing a DNA encoding a polypeptide or a polypeptide into which a mutation is introduced, which comprises the steps of:

(a) producing an antibody-producing cell that produces a polypeptide or a polypeptide into which a mutation is introduced, by the method of any one of [5] to [8] and [11] to [24]; and

(b) isolating a DNA encoding the polypeptide or polypeptide into which a mutation is introduced from the antibody-producing cell produced in step (a).

[30] A method for producing a polypeptide or a polypeptide into which a mutation is introduced, which comprises the steps of:

(a) producing an antibody-producing cell that produces a polypeptide or a polypeptide into which a mutation is introduced, by the method of any one of [5] to [8] and [11] to [24]; and

(b) isolating the polypeptide or polypeptide into which a mutation is introduced from the antibody-producing cell produced in step (a) or a secreted product from the cell.

[31] A method for producing a polypeptide or a polypeptide into which a mutation is introduced, which comprises the steps of:

(a) producing a DNA encoding a polypeptide or a polypeptide into which a mutation is introduced by the method of [29]; and

(b) isolating the polypeptide encoded by the DNA produced in step (a).

[32] An antibody-producing cell in which a region comprising a DNA encoding an antibody variable region is homologously recombined with a DNA construct comprising:

(1) a promoter DNA that functions in the cell;

(2) a DNA that encodes a desired amino acid sequence; and

(3) a DNA that inhibits the production of a polypeptide comprising the desired amino acid sequence and can be removed from the DNA construct.

[33] The cell of [32], wherein the DNA of (3) is a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase.

[34] The cell of [32] or [33], wherein the antibody-producing cell expresses the AID gene.

[35] The cell of [32] or [33], wherein the antibody-producing cell is a cell in which the AID gene expression can be artificially switched on and off.

[36] The cell of [35], wherein the endogenous AID gene is functionally destroyed, and the cell comprises a DNA construct that comprises:

a promoter DNA that functions in the cell; and

a DNA that is located between two site-specific recombinase recognition sequences oriented in opposite directions, and which can be inverted by a site-specific recombinase, and comprises an exogenous AID gene.

[37] The cell of [36], wherein the antibody-producing cell further comprises a DNA construct comprising a promoter DNA that functions in the cell and a DNA encoding a site-specific recombinase, wherein the site-specific recombinase is activated in the presence of an extracellular stimulus and is not activated in the absence of the extracellular stimulus.

[38] The cell of [37], wherein the site-specific recombinase is a fusion protein of a site-specific recombinase with an estrogen receptor or a protein comprising an estrogen-binding domain thereof.

[39] The cell of [38], wherein the estrogen receptor or estrogen-binding domain thereof is a mouse mutant estrogen receptor in which glycine is substituted with arginine at amino acid position 525, or a mouse mutant estrogen-binding domain thereof.

[40] The cell of any one of [32] to [39], wherein the antibody-producing cell has the following properties:

(a) only one of the two alleles of the XRCC3 gene is inactivated in the antibody-producing cell; and

(b) the frequency of introduction of a point mutation is elevated as compared to a cell having the two alleles of the XRCC3 gene.

[41] The cell of [40], wherein the sixth exon is inactivated in either one of the two alleles of the XRCC3 gene.

[42] The cell of any one of [32] to [41], wherein the antibody-producing cell is a B cell.

[43] The cell of [42], wherein the B cell is derived from chicken.

[44] The cell of any one of [33] and [36] to [39], wherein the combination of site-specific recombinase and site-specific recombinase recognition sequence is (i) or (ii) below:

(i) the site-specific recombinase is a Cre recombinase, and the site-specific recombinase recognition sequence is a loxP sequence; and

(ii) the site-specific recombinase is an FLP recombinase, and the site-specific recombinase recognition sequence is an FRT sequence.

[45] The cell of any one of [32] to [44], wherein the polypeptide comprising a desired amino acid sequence comprises an amino acid sequence of an antibody constant region and a desired amino acid sequence.

[46] The cell of [45], wherein the desired amino acid sequence is an amino acid sequence of an antibody variable region.

[47] The cell of any one of [32] to [44], wherein the polypeptide comprising a desired amino acid sequence is an antibody heavy or light chain.

[48] A kit comprising the cell of any one of [32] to [47].

[49] A gene targeting vector comprising a DNA construct that comprises:

(1) a promoter DNA that functions in the cell;

(2) a DNA that comprises a cloning site; and

(3) a DNA that can be removed from the DNA construct and inhibits the production of a polypeptide comprising a desired amino acid sequence encoded by a DNA inserted into the DNA of (2);

wherein the gene targeting vector is used for homologously recombining the DNA construct and a region comprising a DNA encoding an antibody variable region of an antibody-producing cell.

[50] The vector of [49], wherein the DNA of (3) is a DNA that can be removed from the DNA construct by a site-specific recombinase, and inhibits the production of a polypeptide comprising the desired amino acid sequence encoded by the DNA inserted into the DNA of (2), and which is located between two site-specific recombinase recognition sequences oriented in the same orientation, and comprises a promoter DNA and a marker gene that function in the cell.

[51] The vector of [49] or [50], in which a DNA encoding a desired amino acid sequence is inserted into the cloning site.

[52] The vector of any one of [49] to [51], wherein the polypeptide comprising a desired amino acid sequence comprises an amino acid sequence of an antibody constant region and a desired amino acid sequence.

[53] The vector of [52], wherein the desired amino acid sequence is an amino acid sequence of an antibody variable region.

[54] The vector of any one of [49] to [51], wherein the polypeptide comprising a desired amino acid sequence is an antibody heavy or light chain.

[55] A cell comprising the targeting vector of any one of [49] to [54].

[56] A kit comprising the targeting vector of any one of [49] to [54].

[57] A DNA comprising the nucleotide sequence of SEQ ID NO: 7.

[58] A vector comprising the DNA of [57].

[59] A cell comprising the DNA of [57] or the vector of [58].

### Advantageous Effects of Invention

The present invention provides targeting vectors for introducing a DNA encoding a desired amino acid sequence into the antibody variable region gene locus of antibody-producing cells.

According to the present invention, desired polypeptides can be produced under specific conditions by introducing a DNA encoding a polypeptide comprising a desired amino acid sequence into the antibody variable region gene locus of antibody-producing cells.

Furthermore, polypeptides into which mutations are introduced can be produced using the ability of antibody-producing cells to introduce mutations.

The present invention is useful for functional modification of polypeptides.

### BRIEF DESCRIPTION OF DRAWINGS

FIG. 1 presents in schematic diagrams a method for introducing a foreign antibody variable-region gene and a chimeric antibody in which the variable region is substituted. A shows a schematic diagram of the antibody gene locus and a method for introducing a foreign antibody variable region gene. By gene targeting, the antibody gene locus in the DT40 cell genome is modified by replacing the chicken antibody variable region gene with a foreign antibody variable region gene, so that the cell expresses a chimeric antibody comprising the foreign antibody variable region and the chicken antibody constant region. B shows a schematic diagram of the chimeric antibody in which the variable region is substituted. Due to the substitution of the variable region gene, the antibody is expressed as a chimera of the foreign antibody variable region and the chicken antibody constant region.

FIG. 2 presents in diagrams a method for preparing DT40 that produces a chimeric antibody comprising a foreign antibody variable region and the chicken antibody constant region. A shows the structure of a targeting vector. B shows the antibody gene locus structure of the cell after targeting. C shows the antibody gene locus structure after removal of the drug-resistance gene by Cre recombinase, which was activated by treating the cells with 4-hydroxytamoxifen after targeting. D shows an example of the scheme for producing DT40 cells in which a foreign antibody variable region gene is introduced.

FIG. 3 shows a restriction map of an antibody heavy chain variable region gene. Restriction sites marked with asterisk (*) are derived from lambda DASH II.

FIG. 4 presents in diagrams the positions of restriction sites in VH targeting vector 1. A shows the sequence of the chicken antibody heavy chain variable region gene, and the positions of restriction sites into which a foreign antibody variable region gene is inserted. B shows a method for inserting a foreign antibody variable region gene into VH targeting vector 1. The dotted lines with an arrow indicate the regions encoding a signal peptide, VH, and JH, respectively. The vertical arrows indicate the cleavage site of the signal peptide.

FIG. 5 shows a scheme for constructing VH targeting vectors. A shows the XbaI-NotI fragment from the chicken antibody heavy chain gene, which was used to construct VH targeting vectors. B to E show a scheme for constructing VH targeting vector 1. F shows the structure of VH targeting vector 2.

FIG. 6 presents in diagrams the positions of restriction sites in VH targeting vector 2. A shows the sequence of the chicken antibody heavy chain variable region gene and the positions of restriction sites into which a foreign antibody variable region gene is inserted. B shows a method for inserting a foreign antibody variable region gene into VH targeting vector 2. The dotted lines with an arrow indicate the regions encoding a signal peptide, VH, and JH, respectively. The vertical arrows indicate the cleavage site of the signal peptide.

FIG. 7 presents in diagrams the positions of restriction sites in VL targeting vector 1. A shows the sequence of the chicken antibody light chain variable region gene and the positions of restriction sites into which a foreign antibody variable region gene is inserted. B shows a method for inserting a foreign antibody variable region gene into VL targeting vector 1. The dotted lines with an arrow indicate the regions encoding a signal peptide, VL, and JL, respectively. The vertical arrows indicate the cleavage site of the signal peptide.

FIG. 8 shows a scheme for constructing VL targeting vectors. A shows a fragment around the chicken antibody light chain gene, which was used to construct VL targeting vectors. B and C show a scheme for constructing VL targeting vector 1. D shows the structure of VL targeting vector 2.

FIG. 9 presents diagrams showing the positions of restriction sites in VL targeting vector 2. A shows the sequence of the chicken antibody light chain variable region gene and the positions of restriction sites into which a foreign antibody variable region gene is inserted. B shows a method for inserting a foreign antibody variable region gene into VL targeting vector 2. The dotted lines with an arrow indicate the regions encoding a signal peptide, VL, and JL, respectively. The vertical arrows indicate the cleavage site of the signal peptide.

FIG. 10 presents mouse VHT gene targeting in a diagram and photographs. A shows the structure of a mouse VHT gene targeting vector and the genomic structure around the chicken antibody heavy chain variable region gene after targeting. B shows a result of PCR performed to verify the structure of the endogenous antibody heavy chain gene after targeting. VHT is a clone in which the mouse VHT gene was targeted, and the band for the rearranged chain was found to be absent in this clone. SW refers to DT40-SW without targeting. C shows a result of Southern blotting performed to confirm the structure of the endogenous antibody heavy chain gene after targeting. SW is DT40-SW without targeting, while C2 and C3 are clones in which the band for the rearranged chain was found to be absent by PCR. The left pair of C2 and C3 shows the result before 4-hydroxytamoxifen treatment, while the right pair shows the result after 4-hydroxytamoxifen treatment.

FIG. 11 presents charts showing antibody expression in cells in which the mouse VHT gene was targeted. A shows a result of flow cytometry analysis of IgM antibody expression on the cell surface. The cell-surface antibody expression was absent in VHT cells in which the mouse VHT gene was successfully targeted. B shows a result of flow cytometry analysis of IgM antibody expression after 4-hydroxytamoxifen treatment. The treatment with 4-hydroxytamoxifen restored the cell-surface antibody expression in the cells of A. C shows a result of flow cytometry analysis of VHT expression. Cells in which the antibody expression was restored as shown in B expressed the introduced mouse antibody gene (VHT). D shows a result of flow cytometry analysis of antibody expression in the cloned cells (C2 and C3) after 4-hydroxytamoxifen treatment. Stable antibody expression is also observed in the cloned cells after 4-hydroxytamoxifen treatment.

FIG. 12A shows a result of mutation analysis after mutagenesis. This result was obtained by mutation analysis of DT40-SW. The pie chart indicates the total number of clones and the number of mutated clones.

FIG. 12B shows a result of mutation analysis after mutagenesis. This result was obtained by mutation analysis of C2. The underlines indicate the CDR domains of VHT. The pie chart indicates the number of clones with mutations. The table shows mutated nucleotides.

FIG. 12C shows a result of mutation analysis after mutagenesis. This result was obtained by mutation analysis of C3. The underlines indicate the CDR domains of VHT. The pie chart indicates the number of clones with mutations. The table shows mutated nucleotides.

FIG. 13 presents a diagram and photographs showing mouse lambda1 gene targeting. A shows the structure of a mouse lambda gene targeting vector and the genomic structure around the chicken antibody light chain variable region gene after targeting. B shows a result of PCR performed to verify the structure of the endogenous antibody light chain gene after targeting. VHT-lambda1-B4 is a clone in which the gene was targeted. SW refers to DT40-SW without targeting. C shows a result of PCR performed to confirm the insertion of the targeting vector into the chicken antibody light chain gene.

FIG. 14 shows a flow diagram for preparing cells in which the mouse VHT and lambda1 genes were targeted and shift of the antibody expression.

FIG. 15 presents a diagram showing the chimeric antibody expression in cells in which the mouse VHT and lambda1 genes were targeted. Cells in which the mouse VHT and lambda1 genes were targeted (VHT-lambda1 clone B4) were demonstrated to express the antibody. VHT-lambda1 clone B4, DT40-SW cells (negative control), and QM mouse spleen cells (positive control) were analyzed by flow cytometry without (−) or after staining with an antibody against mouse VHT (alpha-Id) or antibody against mouse lambda light chain (alpha-lambda1,2,3).

FIG. 16 shows in a diagram and photograph a result of PCR performed to assess the antibody light chain gene expression in cells in which the mouse VHT and lambda1 genes were targeted (VHT-lambda1B4).

FIG. 17 shows a result of ELISA performed to assess the antibody expression in cells in which the mouse VHT and lambda1 genes were targeted (VHT-lambda1B4). A shows assessment of the expression level of IgM antibody. B shows assessment of the antibody specificity.

FIG. 18 shows the frequency of mutations introduced into the mouse lambda1 antibody in cells in which the mouse lambda1 gene was targeted.

FIG. 19 shows the sequence of a fragment of the upstream region of the antibody heavy chain variable region (SEQ ID NO: 4). This diagram shows the sequence of the upstream region of the antibody heavy chain variable region gene that was identified by the DNA walking method. This sequence includes a newly identified portion alone.

FIG. 20 shows the nucleotide sequence (SEQ ID NO: 7) around the antibody heavy chain variable region gene of DT40. This sequence includes the entire nucleotide sequence of the antibody heavy chain gene whose restriction map is shown in FIG. 3. The underlines (positions 1 to 3120 and 3882 to 7891) show newly identified nucleotide sequences. Positions 1-3223, 5′ upstream region; positions 3224-3269 and 3453-3463, signal peptide-encoding sequence (the sequence of positions 3270-3452 is an intron and is removed by splicing; the sequence of positions 3224-3269 and 3453-3463 are linked together to form the signal peptide sequence); positions 3464-3839, the region of the antibody heavy chain variable region gene, excluding the signal peptide; positions 3840-7931, 3′ downstream region. The VH targeting vectors described in the Examples were prepared using the sequences of positions 1829 to 3223 (from the BamHI site to the nucleotide immediately before the start codon) and 3869 to 6548 (from the SacII site immediately after the JH-encoding region to the XhoI site) as arms, and the sequence of positions 3224 to 3463 as the signal peptide.

FIG. 21 shows the nucleotide sequence of VH targeting vector 1 (SEQ ID NO: 10). This sequence includes the entire nucleotide sequence of the structure shown in FIG. 5E, but not the vector backbone portion. The single underline indicates the signal peptide sequence, and the double underlines indicate the loxP sequences.

FIG. 22 shows the nucleotide sequence of VH targeting vector 2 (SEQ ID NO: 13). This sequence includes the entire nucleotide sequence of the structure shown in FIG. 5F, but not the vector backbone portion. The underline indicates the signal peptide sequence, and the double underlines indicate the loxP sequences.

FIG. 23 shows the nucleotide sequence of VL targeting vector 1 (SEQ ID NO: 18). This sequence includes the entire nucleotide sequence of the structure shown in FIG. 8C, but not the vector backbone portion. The underlines (positions 1869-1914 and 2040-2056) indicate the signal peptide sequence, and the double underlines indicate the loxP sequences. The arm regions homologous to the sequences around the chicken antibody light chain gene are located at positions 1 to 1868 (excluding the signal peptide sequence) and 5011 to 6870.

FIG. 24 shows the nucleotide sequence of VL targeting vector 2 (SEQ ID NO: 20). This sequence includes the entire nucleotide sequence of the structure shown in FIG. 8D, but not the vector backbone portion. The underlines (positions 1869-1914 and 2040-2056) indicate the signal peptide sequence, and the double underlines indicate the loxP sequences. The arm regions homologous to the sequences around the chicken antibody light chain gene are located at positions 1-1868 (excluding the signal peptide sequence) and 5002-6861.

FIG. 25 shows the sequence of the tamoxifen-binding site (SEQ ID NO: 40) of a mutant estrogen receptor. The mutation site is indicated by an underline. Due to the Gto-C nucleotide substitution, the amino acid encoded by the codon is altered from glycine (G) to arginine (R).

FIG. 26 shows the chicken AID cDNA nucleotide sequence used as an AID expression cassette. The underlined portion (positions 6 to 597) shows the proteinencoding region.

FIG. 27 shows the nucleotide sequence of the modified Cre recombinase gene used to fuse it to a mutant estrogen receptor. The stop codon at the end is eliminated from the fusion protein. The underlined portion (positions 4 to 21) shows the nuclear translocation signal derived from the SV40 large T antigen.

FIG. 28 presents a diagram and photographs showing chicken VL gene targeting. A shows the structure of a chicken VL gene targeting vector and the genomic structure around the chicken antibody light chain variable region gene after targeting. B shows a result of PCR performed to assess the structure of the endogenous antibody light chain gene after targeting. cVL-C4 is the targeted clone. C shows a result of PCR performed to assess the insertion of the targeting vector into the chicken antibody light chain gene.

FIG. 29 presents in a diagram shift of expression of the targeted chicken VL gene.

FIG. 30 shows the frequency of mutations introduced into the targeted chicken VL gene.

### DESCRIPTION OF EMBODIMENTS

The present invention relates to methods of homologously recombining a DNA construct and a region comprising a DNA encoding an antibody variable region of an antibody-producing cell, which comprise the steps of introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and which can be removed from the DNA construct.

Herein, the targeting vector may also be referred to as “gene targeting vector”.

The method of homologously recombining a DNA construct and a region comprising a DNA encoding an antibody variable region of an antibody-producing cell may also be referred to as “method for introducing a DNA encoding a desired amino acid sequence into the antibody variable region gene locus of an antibody-producing cell”.

The region comprising a DNA encoding an antibody variable region of an antibody-producing cell can also be referred to as “a DNA in the antibody gene locus”, “a region around a DNA encoding an antibody variable region”, “a DNA region comprising a DNA encoding an antibody variable region and a promoter region located on the 5′ side thereof”, “a region comprising a DNA encoding an antibody variable region and a DNA on the 5′ side thereof”, “a region comprising a DNA encoding an antibody variable region and a DNA on the 3′ side thereof”, “a DNA encoding an antibody variable region of an antibody-producing cell and DNA regions flanking the 5′ and 3′ sides of the DNA”, or the like.

The targeting vectors of the present invention may comprise a DNA homologous to a DNA in the antibody gene locus, in addition to the DNAs of (1) to (3) above. The DNA homologous to a DNA in the antibody gene locus can also be referred to as “a DNA comprising a nucleotide sequence homologous to a region around a DNA encoding an antibody variable region of an antibody-producing cell”, “a DNA comprising a nucleotide sequence homologous to a 5′-side region of a promoter region located on the 5′ side of a DNA encoding an antibody variable region of an antibody-producing cell”, “a DNA comprising a nucleotide sequence homologous to a 3′-side region of a DNA encoding an antibody variable region”, “a DNA homologous to a DNA on the 5′ side of a DNA encoding an antibody variable region”, “a DNA homologous to a DNA on the 3′ side of a DNA encoding an antibody variable region”, “a DNA comprising a nucleotide sequence homologous to DNA regions flanking the 5′ and 3′ sides of a DNA encoding an antibody variable region of an antibody-producing cell”, or the like.

Herein, the “DNA on the 5′ side” can also be referred to as “DNA of the 5′-side upstream region”, and the “DNA on the 3′ side” can also be referred to as “DNA of the 3′-side downstream region”.

When a vector of the present invention is introduced into antibody-producing cells, homologous recombination occurs between a DNA comprising a nucleotide sequence homologous to a region around a DNA encoding an antibody variable region comprised in the vector and a region around the DNA encoding an antibody variable region in the genome of the antibody-producing cells. As a result, the region comprising the DNA encoding an antibody variable region of the antibody-producing cells is replaced with a DNA construct comprising the DNAs of (1) to (3) above.

When the DNA of (1) above is a promoter located on the 5′ side of a DNA encoding an antibody variable region of antibody-producing cells, the DNA of (1) above can also be referred to as “a DNA comprising a nucleotide sequence homologous to a region around a DNA encoding an antibody variable region of antibody-producing cells”. Furthermore, the DNA comprising a nucleotide sequence homologous to a region around a DNA encoding an antibody variable region can also be referred to as “a DNA comprising a nucleotide sequence homologous to DNA regions flanking the 5′ and 3′ sides of a DNA encoding an antibody variable region”. In this case, the methods of the present invention can also be described as methods of homologously recombining a DNA construct and a region comprising a DNA encoding an antibody variable region of an antibody-producing cell, which comprise the step of introducing into the antibody-producing cell a targeting vector comprising a DNA construct that comprises:

(1) a DNA that encodes a desired amino acid sequence; and

(2) a DNA that inhibits the production of a polypeptide comprising the desired amino acid sequence, and can be removed from the DNA construct.

The “DNA comprising a nucleotide sequence homologous to a DNA region around a DNA encoding an antibody variable region of an antibody-producing cell” can also be referred to as “vector arm” or “arm”. In particular, the “DNA homologous to a DNA on the 5′ side of a DNA encoding an antibody variable region of an antibody-producing cell” can also be referred to as “upstream arm”, “5′-side arm, or “left arm”. Furthermore, the “DNA homologous to a DNA on the 3′ side of a DNA encoding an antibody variable region of an antibody-producing cell” can also be referred to as “downstream arm”, “3′-side arm”, or “right arm”. It is generally thought that longer arms are preferred; however, the arms are only required to have a length normally used for gene targeting (A. Joyner, “Gene Targeting”, IRL Press Practical Approach series, Oxford Univ. Press). For example, without limitation, the length of the right and left arms may be 1 kb or longer, and the combined length may be 3 kb or more. Meanwhile, the full length of the vectors is preferably 12 kbp or less, but is not limited thereto (J.-M. Buesrstedde and S. Takeda, eds., Subcellular Biochemistry Volume 40: Reviews and Protocols in DT40 Research, Springer (2006)).

In the present invention, the “DNA homologous to a DNA on the 5′ side of a DNA encoding an antibody variable region of an antibody-producing cell” includes both DNA that has a promoter located on the 5′ side of a DNA encoding an antibody variable region of an antibody-producing cell and DNA that has not.

When the “DNA homologous to a DNA on the 5′ side of a DNA encoding an antibody variable region of an antibody-producing cell” does not have a promoter located on the 5′ side of the DNA encoding an antibody variable region of an antibody-producing cell, the DNA can also be referred to as “a DNA comprising a nucleotide sequence homologous to a 5′-side region of a promoter region located on the 5′ side of a DNA encoding an antibody variable region of an antibody-producing cell”.

In the present invention, the “DNA homologous to a DNA on the 3′ side of a DNA encoding an antibody variable region of an antibody-producing cell” includes both DNA that has a DNA encoding an antibody constant region of an antibody-producing cell and DNA that has not.

In the present invention, it is not necessary for the nucleotide sequences of arms or vector arms to be completely homologous (identical) to a DNA region around a DNA encoding an antibody variable region of antibody-producing cells, and they are only required to have a degree of similarity that allows homologous recombination.

A functional promoter DNA that functions in an antibody-producing cell can be appropriately selected according to the type of antibody-producing cell. The promoter DNAs that function in antibody-producing cells include, but are not limited to, the chicken beta-actin promoter and human elongation factor 1alpha(EF-1alpha) promoter (Yang, S. Y. et al., J. Exp. Med. 203:2919-2928 (2006)). Examples of virus promoter DNAs include, but are not limited to, cytomegalovirus (CMV) promoter (Kanayama, N., et al. Nucleic Acids Res. 34, e10 (2006)) and Rous sarcoma virus (RSV) promoter (Arakawa, H., et al. Nucleic Acids Res. 36, e1 (2008)).

The promoter DNAs that function in an antibody-producing cell include promoters derived from the antibody-producing cell and antibody gene promoter DNAs located on the 5′ side of the DNA encoding an antibody variable region.

Herein, the “DNA encoding a desired amino acid sequence” can also be referred to as a “DNA encoding an arbitrary amino acid sequence” or a “DNA encoding an amino acid sequence of interest”.

The desired amino acid sequences include, but are not limited to, amino acid sequences of antibody variable regions (antibody heavy-chain variable regions and antibody light-chain variable regions), amino acid sequences of enzymes, amino acid sequences of receptors, and artificial peptide sequences.

When a DNA encoding a desired amino acid sequence does not contain a signal peptide-encoding DNA, a DNA encoding a signal peptide may be inserted on the 5′ side of the DNA encoding a desired amino acid sequence in the gene targeting vector in order to enhance cell-surface expression or secretion of the polypeptide encoded by the DNA encoding a desired amino acid sequence. A signal peptide-encoding DNA can be inserted at a site so that the two peptide-encoding regions are linked in-frame in the mRNA transcribed from the signal peptide-encoding DNA and the DNA encoding a desired amino acid sequence. For example, an intron may be inserted between the signal peptide-encoding DNA and the DNA encoding a desired amino acid sequence. Alternatively, as described below, the DNA of (3) above may be inserted between the signal peptide-encoding DNA and the DNA encoding a desired amino acid sequence. There is no limitation on the signal peptide-encoding DNA, as long as it has the ability to express a polypeptide on the surface of antibody-producing cells or to secret the polypeptide from antibody-producing cells. The signal peptide-encoding DNAs include those derived from the antibody-producing cells and DNAs encoding a signal peptide on the 5′ side of an antibody variable region.

Such signal peptides include, but are not limited to, signal peptides of chicken antibody heavy or light chains, signal peptides of antibody heavy or light chains of mammals such as humans and mice, and signal peptides of avian or mammalian cytokines or growth factors that are secreted to the outside of cells.

The DNAs encoding a desired amino acid sequence may originate from any species. The DNAs may be derived from the same species from which the antibody-producing cells are derived, or may be derived from a different species. Furthermore, the DNAs may be those encoding an artificially modified polypeptide such as a chimeric polypeptide.

The “polypeptides comprising a desired amino acid sequence” include a polypeptide comprising a desired amino acid sequence and an amino acid sequence of an antibody constant region, but are not limited thereto.

The “polypeptides comprising a desired amino acid sequence” include, but are not limited to, polypeptides (chimeric polypeptides) comprising the following amino acid sequences:

an amino acid sequence of an antibody variable region (antibody heavy-chain variable region or antibody light-chain variable region) and an amino acid sequence of an antibody constant region (antibody heavy-chain constant region or antibody light-chain constant region);

an amino acid sequence of an enzyme and an amino acid sequence of an antibody constant region (antibody heavy-chain constant region or antibody light-chain constant region); or

an amino acid sequence of a receptor and an amino acid sequence of an antibody constant region (antibody heavy-chain constant region or antibody light-chain constant region).

Herein, the “DNA that inhibits the production of a polypeptide comprising a desired amino acid sequence” refers to a DNA that inhibits the normal production of a polypeptide comprising a desired amino acid sequence by blocking any of the following steps in the presence of the DNA:

transcription of a DNA encoding a polypeptide comprising a desired amino acid sequence;

splicing of an mRNA containing the region encoding a polypeptide comprising a desired amino acid sequence;

translation of the mRNA; and

processing (for example, folding) of a polypeptide comprising a desired amino acid sequence.

The DNA of (3) above can also be described as “a DNA that inhibits the production of a polypeptide comprising a desired amino acid sequence and is located between two site-specific recombinase recognition sequences oriented in the same direction, and which comprises a promoter DNA and a marker gene that function in the cell, and can be removed from the DNA construct by a site-specific recombinase”.

The marker genes of the present invention preferably contain a poly A (adenine) addition sequence at their 3′ end. The poly A addition sequence terminates the transcription of marker genes.

Those skilled in the art can think of various promoters and select an appropriate promoter. Such promoters include, but are not limited to, for example, the beta-actin promoter, immunoglobulin promoter, cytomegalovirus promoter, CAG promoter, and EF1alpha promoter.

Selection marker genes include, but are not limited to, antibiotic-resistance genes such as the neomycin phosphotransferase gene, blasticidin S deaminase gene, puromycin N-acetyltransferase gene, histidinol dehydrogenase gene, hygromycin B phosphotransferase gene, and xanthine-guanine phosphoribosyltransferase gene; fluorescent protein genes such as GFP and DsRed; and genes of chromogenic enzymes such as beta-galactosidase (lacZ) and beta-lactamase.

It is preferable to operably link a promoter and a marker gene together so that the marker gene can be expressed by the promoter.

Herein, “operably linked” means that a marker gene is linked to a promoter DNA that functions in an antibody-producing cell, so that the expression of the marker gene is induced by binding of transcriptional factors to the promoter DNA. Thus, when a marker gene is linked with another gene and a fusion protein with its gene product is formed, and expression of the fusion protein is induced as a result of binding of transcriptional factors to the promoter region of the marker gene, this is also included in the meaning of “operably linked”.

The combinations of a site-specific recombinase and a site-specific recombinase recognition sequence include a combination of Cre recombinase and loxP and a combination of FLP recombinase and FRT, but are not limited thereto. In the present invention, the site-specific recombinase recognition sequence may be a mutant sequence, as long as it is recognized by a site-specific recombinase.

For the loxP sequence, see the following documents:
Hoess et al., P1 site-specific recombination: nucleotide sequence of the recombining sites. Proc Natl Acad Sci USA (1982) vol. 79 (11) pp. 3398-402
Hoess and Abremski, Interaction of the bacteriophage P1 recombinase Cre with the recombining site loxP. Proc Natl Acad Sci USA (1984) vol. 81 (4) pp. 1026-9 For the sequence of FLP recombinase, see the following document:
Hartley and Donelson, Nucleotide sequence of the yeast plasmid. Nature (1980) vol. 286 (5776) pp. 860-865

For the FRT sequence, see the following documents:
Hartley and Donelson, Nucleotide sequence of the yeast plasmid. Nature (1980) vol. 286 (5776) pp. 860-865
Andrews et al., The FLP recombinase of the 2 micron circle DNA of yeast: interaction with its target sequences. Cell (1985) vol. 40 (4) pp. 795-803
Gronostaj ski and Sadowski, Determination of DNA sequences essential for FLPmediated recombination by a novel method. J Biol Chem (1985) vol. 260 (22) pp. 12320-7
Senecoff et al., The FLP recombinase of the yeast 2-micron plasmid: characterization of its recombination site. Proc Natl Acad Sci USA (1985) vol. 82 (21) pp. 7270-4

There is no limitation on the gene targeting vectors of the present invention, as long as they allow homologous recombination between the above-described DNA construct and a region comprising a DNA encoding an antibody variable region of antibody-producing cells. The gene targeting vectors of the present invention include, for example, the vectors of (A) and (B) below.

(A) A gene targeting vector used for homologously recombining a DNA construct and a region comprising a DNA encoding an antibody variable region of an antibody-producing cell, wherein the vector comprises the DNA construct comprising the DNAs of (1) to (3) below from the 5′ to 3′ end of the vector DNA chain:

(1) a promoter DNA that functions in the antibody-producing cell;

(2) a DNA that inhibits the production of a polypeptide comprising a desired amino acid sequence, and can be removed from the DNA construct; and

(3) a DNA that encodes the desired amino acid sequence.

The vector of (A) may have a DNA encoding a signal peptide on the 5′ side of the DNA encoding a desired amino acid sequence. In this case, the vector may have a signal peptide-encoding DNA between the DNA of (2) and the DNA of (3). Alternatively, the vector may have a signal peptide-encoding DNA between the DNA of (1) and the DNA of (2). However, the vector is not limited to these examples.

(B) A gene targeting vector used for homologously recombining a DNA construct and a region comprising a DNA encoding an antibody variable region of an antibody-producing cell, wherein the vector comprises the DNA construct comprising the DNAs of (1) to (3) below from the 5′ to 3′ end of the vector DNA chain:

(1) a promoter DNA that functions in the antibody-producing cell;

(2) a DNA that encodes a desired amino acid sequence; and

(3) a DNA that inhibits the production of a polypeptide comprising the desired amino acid sequence, and can be removed from the DNA construct.

The vector of (B) may also have a DNA encoding a signal peptide on the 5′ side of the DNA encoding a desired amino acid sequence. In this case, the vector may have a signal peptide-encoding DNA between the DNA of (1) and the DNA of (2). However, the vector is not limited to this example.

The gene targeting vector of (A) above can also be described as a gene targeting vector for homologously recombining a DNA construct and a region comprising a DNA encoding an antibody variable region of an antibody-producing cell, wherein the vector comprises a DNA construct comprising the DNAs of (i) to (iii) below from the 5′ to 3′ end of the vector DNA chain:

(i) a DNA comprising a DNA homologous to a 5′-side region of a DNA encoding an antibody variable region in the genomic DNA of an antibody-producing cell;

(ii) a DNA encoding a desired amino acid sequence; and

(iii) a DNA comprising a DNA homologous to a 3′-side region of the DNA encoding an antibody variable region in the genomic DNA of an antibody-producing cell,

wherein the DNA of (i) comprises (alpha) and (beta) below from the 5′ to 3′ end of the vector DNA chain:

(alpha) a promoter DNA that functions in the antibody-producing cell; and

(beta) a DNA that inhibits the production of a polypeptide comprising the desired amino acid sequence and can be removed from the DNA construct.

The above gene targeting vector may have a DNA encoding a signal peptide on the 5′ side (more upstream) of the DNA of (ii). More specifically, the gene targeting vector may have a signal peptide-encoding DNA between the DNA of (alpha) and the DNA of (beta), or between the DNA of (beta) and the DNA of (ii). However, the vector is not limited to these examples.

The DNA of (iii) may have a DNA encoding an antibody constant region.

The homologous DNA of (i) can be recombined with a DNA on the 5′ side of a DNA encoding an antibody variable region of antibody-producing cells. When the target of the gene targeting vector is a heavy chain variable region, the 5′-side DNAs include, but are not limited to, for example, a DNA fragment that starts at the 5′ terminal nucleotide of a DNA encoding the signal peptide of an antibody heavy-chain variable region in the chicken antibody heavy chain gene locus and extends up to the first BamHI, or first XhoI, or first XbaI site located in the upstream (5′-side) DNA region of the starting point.

Alternatively, when the target of the gene targeting vector is a light-chain variable region, the 5′-side DNAs include, but are not limited to, for example, a DNA fragment that starts at the 5′ terminal nucleotide of a DNA encoding a signal peptide of the antibody light chain variable region in the chicken antibody light chain gene locus and extends up to the first SacI or first BamHI site located in the upstream (5′-side) DNA region of the starting point.

When a 5′-side DNA is prepared using a DNA encoding a signal peptide of an antibody variable region as the starting point, an antibody gene promoter is already included in the 5′-side DNA. In this case, it is not necessary to insert a promoter DNA into the vector.

Furthermore, the homologous DNA of (iii) can be recombined with a DNA on the 3′ side of a DNA encoding an antibody variable region of antibody-producing cells. When the target of the gene targeting vector is a heavy-chain variable region, the 3′-side DNAs include, but are not limited to, for example, a DNA fragment that starts at the 3′ terminal nucleotide of a DNA encoding an antibody heavy-chain variable region in the chicken heavy chain antibody gene locus, and extends to the first XhoI or first ClaI site located in the downstream (3′-side) DNA region of the starting point.

Alternatively, when the target of the gene targeting vector is a light-chain variable region, the 3′-side DNAs include, but are not limited to, for example, a DNA fragment that starts at the 3′ terminal nucleotide of a DNA encoding the antibody light chain variable region in the chicken light chain antibody gene locus, and extends to the first ClaI or first EcoRI site located in the downstream (3′-side) DNA region of the starting point of the fragment.

The gene targeting vector of (A) above can also be described as a gene targeting vector for homologously recombining a DNA construct and a region comprising the DNA encoding an antibody variable region of an antibody-producing cell, which comprises a DNA construct comprising the DNAs of (i) to (v) below from the 5′ to 3′ end of the vector DNA chain:

(i) a DNA homologous to a region on the 5′ side of a DNA encoding an antibody variable region in the genomic DNA of an antibody-producing cell;

(ii) a promoter DNA that functions in the antibody-producing cell;

(iii) a DNA that inhibits the production of a polypeptide comprising a desired amino acid sequence, and can be removed from the DNA construct;

(iv) a DNA that encodes the desired amino acid sequence; and

(v) a DNA homologous to a region on the 3′ side of the DNA encoding an antibody variable region in the genomic DNA of the antibody-producing cell.

The above gene targeting vector may have a DNA encoding a signal peptide on the 5′ side of the DNA of (iv).

The DNA of (v) may have a DNA encoding an antibody constant region.

Gene targeting vectors of the present invention that target a heavy-chain variable region can be designed based on the DNA fragments listed below, each of which contains a region around the chicken antibody heavy-chain gene locus. However, the gene targeting vectors are not limited to these examples.

B amHI-XhoI fragment

BamHI-ClaI fragment

XhoI-XhoI fragment

XhoI-ClaI fragment

XbaI-XhoI fragment

XbaI-ClaI fragment

Gene targeting vectors of the present invention that target a light-chain variable region can be designed based on the DNA fragments listed below, each of which contains a region around the chicken antibody light-chain gene locus. However, the gene targeting vectors are not limited to these examples.

SacI-ClaI fragment

BamHI-EcoRI fragment

SacI-ClaI fragment

BamHI-EcoRI fragment

In the present invention, the restriction sites are not limited to those naturally occurring in the chicken antibody gene locus. DNA fragments containing desired restriction sites can be obtained by PCR amplification using primers to which the restriction sites are added. The restriction sites of the present invention also include the thus produced sites.

A gene targeting vector of the present invention that targets a heavy-chain variable region can also be described as a gene targeting vector comprising a DNA construct that comprises:

a DNA fragment comprising the nucleotide sequence (SEQ ID NO: 44) from the BamHI site on the 5′ side of the coding region of an antibody variable region to the nucleotide immediately before the start codon in the chicken heavy chain antibody gene locus;

a DNA fragment comprising the SacII-XhoI fragment (SEQ ID NO: 45) on the 3′ side of the coding region of the antibody variable region; and

a DNA fragment comprising the DNA of (ii) above. The DNA fragment comprising the nucleotide sequence of SEQ ID NO: 44 includes, but is not limited to, a DNA fragment comprising a further 5′-side region. The DNA fragment comprising the nucleotide sequence of SEQ ID NO: 45 includes, but is not limited to, a DNA fragment comprising a further 3′-side region.

A gene targeting vector of the present invention that targets a light-chain variable region can also be described as a gene targeting vector comprising a DNA construct that comprises:

a DNA fragment comprising the nucleotide sequence (SEQ ID NO: 46) from the SacI site to the nucleotide immediately before the start codon in the chicken light-chain antibody gene locus;

a DNA fragment comprising the nucleotide sequence of SEQ ID NO: 47; and

a DNA fragment comprising the DNA of (ii) above. The DNA fragment comprising the nucleotide sequence of SEQ ID NO: 46 includes, but is not limited to, a DNA fragment comprising a further 5′-side region. The DNA fragment comprising the nucleotide sequence of SEQ ID NO: 47 includes, but is not limited to, a DNA fragment comprising a further 3′-side region.

A gene targeting vector of the present invention may have all of the characteristics described above. The gene targeting vector of the present invention of (A) above can be described as a gene targeting vector that targets a heavy chain variable region, specifically, as a gene targeting vector used for homologously recombining a DNA construct and a region comprising a DNA encoding an antibody heavy-chain variable region of an antibody-producing cell, wherein the vector comprises a DNA construct comprising the DNAs of (i) to (iii) below from the 5′ to 3′ end of the vector DNA chain:

(i) a DNA comprising a DNA homologous to the 5′ side of a DNA encoding an antibody heavy-chain variable region in the genomic DNA of an antibody-producing cell;

(ii) a DNA encoding a desired amino acid sequence; and

(iii) a DNA comprising a DNA homologous to the 3′ side of the DNA encoding an antibody heavy-chain variable region in the genomic DNA of the antibody-producing cell;

wherein the DNA of (i) comprises (alpha) and (beta) below from the 5′ to 3′ end of the vector DNA chain:

(alpha) a promoter DNA that functions in the antibody-producing cell; and

(beta) a DNA that inhibits the production of a polypeptide comprising the desired amino acid sequence, and can be removed from the DNA construct;

The gene targeting vector of the present invention of (A) above can be described as a gene targeting vector that targets a light-chain variable region, specifically, as a gene targeting vector used for homologously recombining a DNA construct and a region comprising a DNA encoding an antibody light-chain variable region of an antibody-producing cell, wherein the vector comprises a DNA construct comprising the DNAs of (i) to (iii) below from the 5′ to 3′ end of the vector DNA chain:

(i) a DNA comprising a DNA homologous to the 5′ side of a DNA encoding an antibody light-chain variable region in the genomic DNA of an antibody-producing cell;

(ii) a DNA encoding a desired amino acid sequence; and

(iii) a DNA comprising a DNA homologous to the 3′ side of the DNA encoding an antibody light-chain variable region in the genomic DNA of the antibody-producing cell;

wherein the DNA of (i) comprises (alpha) and (beta) below from the 5′ to 3′ end of the vector DNA chain:

(alpha) a promoter DNA that functions in the antibody-producing cell; and

(beta) a DNA that inhibits the production of a polypeptide comprising the desired amino acid sequence, and can be removed from the DNA construct.

The above gene targeting vectors that target a heavy-chain or light-chain variable region may have a DNA encoding a signal peptide on the 5′-side of the DNA of (ii). More specifically, the vectors may have a signal peptide-encoding DNA between the DNA of (alpha) and the DNA of (beta), or between the DNA of (beta) and the DNA of (ii). However, the vectors are not limited to these examples.

The DNA of (iii) may have a DNA encoding an antibody constant region.

The gene targeting vector of (B) above can also be referred to as a gene targeting vector for homologously recombining a DNA construct and a region comprising a DNA encoding an antibody variable region of an antibody-producing cell, wherein the vector comprises a DNA construct comprising the DNAs of (i) to (iii) from the 5′ end to 3′ end of the vector DNA chain:

(i) a DNA comprising a DNA homologous to the 5′ side of a DNA encoding an antibody variable region in the genomic DNA of an antibody-producing cell;

(ii) a DNA encoding a desired amino acid sequence; and

(iii) a DNA comprising a DNA homologous to the 3′ side of the DNA encoding an antibody variable region in the genomic DNA of the antibody-producing cell;

wherein the DNA of (i) comprises (alpha) a promoter DNA that functions in the antibody-producing cell; and

the DNA of (iii) comprises (beta) a DNA that inhibits the production of a polypeptide comprising the desired amino acid sequence, and can be removed from the DNA construct; and

the DNA of (alpha) is operably linked to the DNA of (ii).

The homologous DNAs of (i) and (iii) are as described above.

The above gene targeting vectors may have a DNA encoding a signal peptide on the 5′ side of the DNA of (ii). More specifically, the gene targeting vectors may have a signal peptide-encoding DNA between the DNA of (alpha) and the DNA of (ii). However, the gene targeting vectors is not limited to this example.

The DNA of (iii) may have a DNA encoding an antibody constant region.

The gene targeting vector of (B) above can also be described as a gene targeting vector used for homologously recombining a DNA construct and a region comprising a DNA encoding an antibody variable region of an antibody-producing cell, wherein the vector comprises a DNA construct comprising the DNAs of (i) to (v) from the 5′ end to 3′ end of the vector DNA chain:

(i) a DNA homologous to the 5′ side of a DNA encoding an antibody variable region in the genomic DNA of an antibody-producing cell;

(ii) a promoter DNA that functions in the antibody-producing cell;

(iii) a DNA that encodes a desired amino acid sequence;

(iv) a DNA that inhibits the production of a polypeptide comprising the desired amino acid sequence, and can be removed from the DNA construct; and

(v) a DNA homologous to the 3′ side of the DNA encoding an antibody variable region in the genomic DNA of the antibody-producing cell.

The above gene targeting vector may have a DNA encoding a signal peptide on the 5′ side of the DNA of (iii).

The DNA of (v) may have a DNA encoding an antibody constant region.

The gene targeting vector described above in (B) above can also be described as a gene targeting vector that targets a heavy chain variable region, specifically, as a gene targeting vector for homologously recombinating a DNA construct and a region comprising a DNA encoding an antibody heavy chain variable region of an antibody-producing cell, wherein the vector comprises a DNA construct comprising the DNAs of (i) to (iii) from the 5′ end to 3′ end of the vector DNA chain:

(i) a DNA comprising a DNA homologous to the 5′ side of a DNA encoding an antibody heavy chain variable region in the genomic DNA of an antibody-producing cell;

(ii) a DNA encoding a desired amino acid sequence; and

(iii) a DNA comprising a DNA homologous to the 3′ side of the DNA encoding an antibody heavy chain variable region in the genomic DNA of the antibody-producing cell;

wherein the DNA of (i) comprises (alpha) a promoter DNA that functions in the antibody-producing cell; and

the DNA of (iii) comprises (beta) a DNA that inhibits the production of a polypeptide comprising the desired amino acid sequence, and can be removed from the DNA construct; and

the DNA of (alpha) is operably linked to the DNA of (ii).

The gene targeting vector described above in (B) above can also be described as a gene targeting vector that targets a light chain variable region, specifically, as a gene targeting vector for homologously recombining a DNA construct and a region comprising a DNA encoding an antibody light chain variable region of an antibody-producing cell, wherein the vector comprises a DNA construct comprising the DNAs of (i) to (iii) from the 5′ end to 3′ end of the vector DNA chain:

(i) a DNA comprising a DNA homologous to the 5′ side of a DNA encoding an antibody light-chain variable region in the genomic DNA of an antibody-producing cell;

(ii) a DNA encoding a desired amino acid sequence; and

(iii) a DNA comprising a DNA homologous to the 3′ side of the DNA encoding an antibody light-chain variable region in the genomic DNA of the antibody-producing cell;

wherein the DNA of (i) comprises (alpha) a promoter DNA that functions in the antibody-producing cell; and

the DNA of (iii) comprises (beta) a DNA that inhibits the production of a polypeptide comprising a desired amino acid sequence, and can be removed from the DNA construct; and

the DNA of (alpha) is operably linked to the DNA of (ii).

The above gene targeting vectors that target a heavy-chain or light-chain variable region may have a DNA encoding a signal peptide on the 5′ side of the DNA of (ii). More specifically, the gene targeting vectors may have the DNA encoding a signal peptide between the DNA of (alpha) and the DNA of (ii). However, the gene targeting vectors are not limited to this example.

The DNA of (iii) may have a DNA encoding an antibody constant region.

The vector of (B) above may additionally comprise a splicing donor consensus sequence at the 3′ end of the DNA encoding a desired amino acid sequence. Such splicing donor consensus sequences include “AG GTRAGT” (R refers to A or G; the underlined portion is an intron sequence), but are not limited thereto.

When a splicing donor consensus sequence is linked to a DNA encoding a desired amino acid sequence, nucleotides need to be appropriately added or deleted before the splicing donor consensus sequence so that the region encoding the desired amino acid sequence is in-frame with the region encoding an antibody constant region in the mRNA after splicing.

In the vector of (B) above, the DNA encoding a desired amino acid sequence (the DNA of (ii)) is preferably as close as possible to the DNA (the DNA of (beta)) that inhibits the production of a polypeptide comprising the desired amino acid sequence, and can be removed from the DNA construct. Furthermore, when the DNA of (beta) comprises a marker gene, it preferably has a poly A addition sequence at the 3′ end of the marker gene.

The present invention provides the gene targeting vectors described above. The present invention also provides targeting vectors that have a cloning site instead of the DNA encoding a desired amino acid sequence in the above targeting vectors. More specifically, the present invention provides gene targeting vectors for homologously recombining a DNA construct and a region comprising a DNA encoding an antibody variable region of an antibody-producing cell, wherein the vector comprise a DNA construct comprising:

(1) a promoter DNA that functions in the cell;

(2) a DNA that comprises a cloning site; and

(3) a DNA that inhibits the production of a polypeptide comprising a desired amino acid sequence encoded by a DNA inserted into the DNA of (2), and which can be removed from the DNA construct.

The DNA of (3) can also be described as a DNA that can be removed from the DNA construct by a site-specific recombinase, and inhibits the production of a polypeptide comprising a desired amino acid sequence encoded by a DNA inserted into the DNA of (2), and which is located between two site-specific recombinase recognition sequences oriented in the same direction, and comprises a promoter DNA and a marker gene that function in the cell.

The marker gene may have a poly A sequence at its 3′ end.

A DNA encoding a desired amino acid sequence may be inserted into the cloning site of the gene targeting vectors of the present invention.

Such cloning sites include, but are not limited to, multicloning sites.

Examples of vectors in which a DNA encoding a desired amino acid sequence (the DNA of (ii)) in the vector of (B) above is substituted with a cloning site are shown hereinbelow. However, the vectors of the present invention are not limited to these examples.

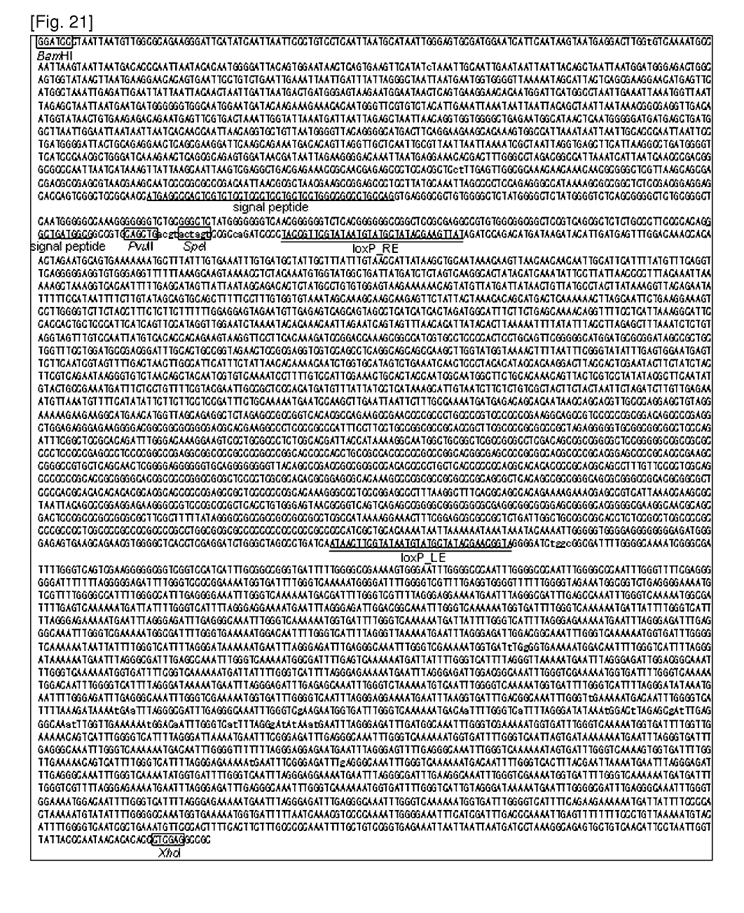

SEQ ID NO: 10 (FIG. 21) is an example of the nucleotide sequence of a gene targeting vector of the present invention that targets a heavy-chain variable region. Specifically, the present invention provides targeting vectors that comprise a DNA construct comprising the nucleotide sequence of SEQ ID NO: 10 (in particular, the nucleotide sequence of positions 1 to 7277).

In FIG. 21 (SEQ ID NO: 10), the nucleotides of positions 1 to 1395 correspond to a DNA sequence homologous to a DNA on the 5′ side of a DNA encoding an antibody variable region of an antibody-producing cell (a sequence containing a promoter until immediately before the start codon of the DNA encoding an antibody variable region);

the nucleotides of positions 1396 to 1441 correspond to a DNA encoding a signal peptide of the antibody variable region;

the nucleotides of positions 1442 to 1624 correspond to an intron DNA;

the nucleotides of positions 1625 to 1635 correspond to a DNA encoding the signal peptide of the antibody variable region;

the nucleotides of positions 1671 to 1704 correspond to the DNA sequence of loxP_RE (TACCGTTCGTATAATGTATGCTATACGAAGTTAT (SEQ ID NO: 37)); the nucleotides of positions 1711 to 2697 correspond to a DNA comprising a poly A addition sequence of a marker;

the nucleotides of positions 2720 to 3142 correspond to a DNA sequence of the marker gene;

the nucleotides of positions 3186 to 4527 correspond to a promoter sequence of the marker gene;

the nucleotides of positions 4555 to 4588 correspond to the DNA sequence of loxP_LE (ATAACTTCGTATAATGTATGCTATACGAACGGTA (SEQ ID NO: 38));

and the nucleotides of positions 4598 to 7277 correspond to a DNA sequence homologous to a DNA on the 3′ side of the DNA encoding an antibody variable region of the antibody-producing cell.

The nucleotides of positions 1442 to 1624 (intron DNA) are removed by splicing, and thus the nucleotides of positions 1397 to 1441 are linked to the nucleotides of positions 1625 to 1635 to form the signal peptide sequence.

As an example, the targeting vector described above uses a mutant loxP sequence (Albert H., et al., Plant J. 7:649 (1995); Araki K., et al., Nucleic Acids Res. 25:868 (1997)).

The present invention uses a marker gene for DT40 (Arakawa H., BMC Biotechnol. 1:7 (2001)). The marker gene for DT40 is described above.

In the above vector, the DNA encoding a desired amino acid sequence is inserted between the DNA encoding the signal peptide and the DNA sequence of loxP_RE.

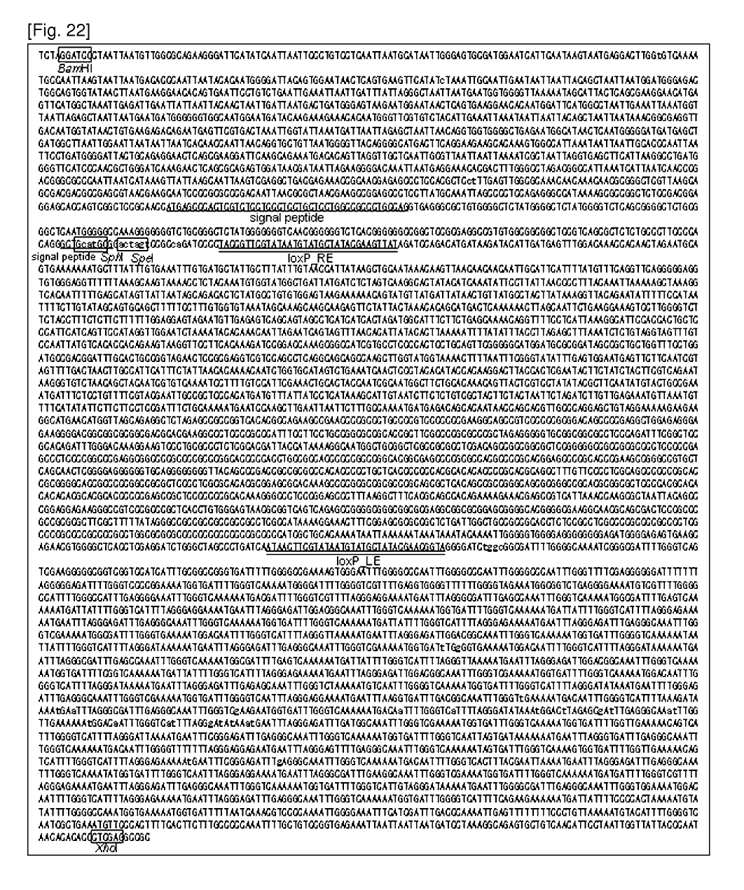

Furthermore, an example of the nucleotide sequence of a gene targeting vector of the present invention that targets a heavy chain variable region is shown in SEQ ID NO: 13 (FIG. 22). Specifically, the present invention provides targeting vectors comprising a DNA construct that comprises the nucleotide sequence of SEQ ID NO: 13 (in particular, the nucleotide sequence of positions 5 to 7266).

In FIG. 22 (SEQ ID NO: 13),

the nucleotides of positions 5 to 1399 correspond to a DNA sequence homologous to a DNA on the 5′ side of a DNA encoding an antibody variable region of an antibody-producing cell (a sequence containing a promoter until immediately before the start codon of the DNA encoding an antibody variable region);

the nucleotides of positions 1400 to 1445 correspond to a DNA encoding a signal peptide of the antibody variable region;

the nucleotides of positions 1446 to 1628 correspond to an intron DNA;

the nucleotides of positions 1629 to 1639 correspond to a DNA encoding the signal peptide of the antibody variable region;

the nucleotides of positions 1660 to 1693 correspond to the DNA sequence of loxP_RE (TACCGTTCGTATAATGTATGCTATACGAAGTTAT (SEQ ID NO: 37)); the nucleotides of positions 1700 to 2686 correspond to a DNA comprising a poly A addition sequence of a marker gene;

the nucleotides of positions 2709 to 3131 correspond to a DNA sequence of the marker gene sequence;

the nucleotides of positions 3175 to 4516 correspond to a promoter sequence of the marker gene;

the nucleotides of positions 4544 to 4577 correspond to the DNA sequence of loxP_LE (ATAACTTCGTATAATGTATGCTATACGAACGGTA (SEQ ID NO: 38));

and the nucleotides of positions 4587 to 7266 correspond to a DNA sequence homologous to a DNA on the 3′ side of the DNA encoding an antibody variable region of the antibody-producing cell.

The nucleotides of positions 1446 to 1628 (intron DNA) are removed by splicing, and thus the nucleotides of positions 1400 to 1445 are linked to the nucleotides of positions 1629 to 1639 to form the signal peptide sequence.

In the above vector, the DNA encoding a desired amino acid sequence is inserted between the signal peptide-encoding DNA and the DNA sequence of loxP_RE.

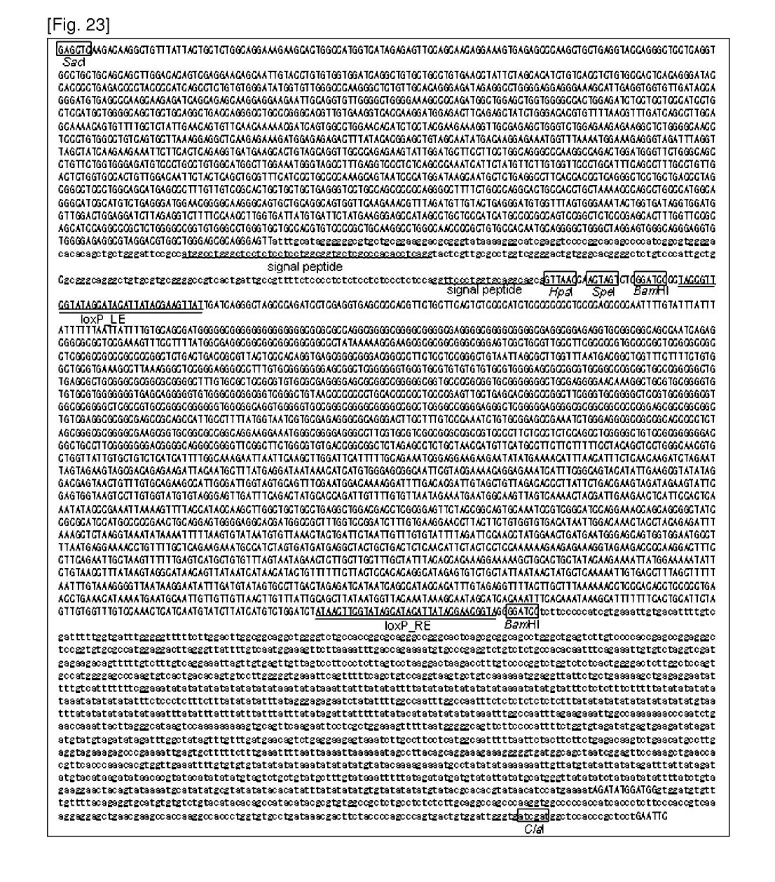

Furthermore, an example of the nucleotide sequence of a gene targeting vector of the present invention that targets a light-chain variable region is shown in SEQ ID NO: 18 (FIG. 23). Specifically, the present invention provides targeting vectors comprising a DNA construct comprising the nucleotide sequence of SEQ ID NO: 18 (in particular, the nucleotide sequence of positions 1 to 6870).

In FIG. 23 (SEQ ID NO: 18),

the nucleotides of positions 1 to 1868 correspond to a DNA sequence homologous to a DNA on the 5′ side of a DNA encoding an antibody variable region of an antibody-producing cell (a sequence containing a promoter until immediately before the start codon of the DNA encoding an antibody variable region);

the nucleotides of positions 1869 to 1914 correspond to a DNA encoding a signal peptide of the antibody variable region;

the nucleotides of positions 1915 to 2039 correspond to an intron DNA;

the nucleotides of positions 2040 to 2056 correspond to a DNA encoding the signal peptide of the antibody variable region;

the nucleotides of positions 2085 to 2118 correspond to the DNA sequence of loxP_LE;

the nucleotides of positions 2146 to 3487 correspond to a promoter sequence of a marker gene;

the nucleotides of positions 3531 to 3953 correspond to a DNA sequence of the marker gene;

the nucleotides of positions 3976 to 4962 correspond to a DNA sequence containing a poly A addition sequence of the marker gene;

the nucleotides of positions 4969 to 5002 correspond to the DNA sequence of loxP_RE; and

the nucleotides of positions 5011 to 6870 correspond to a DNA sequence homologous to a DNA on the 3′ side of the DNA encoding the antibody variable region of the antibody-producing cell.

The nucleotides of positions 1915-2039 (intron DNA) are removed by splicing, and thus the nucleotides of positions 1869 to 1914 are linked to the nucleotides of positions 2040 to 2056 to form the signal peptide sequence,

In the above vector, the DNA encoding a desired amino acid sequence is inserted between the signal peptide-encoding DNA and the DNA sequence of loxP_LE.

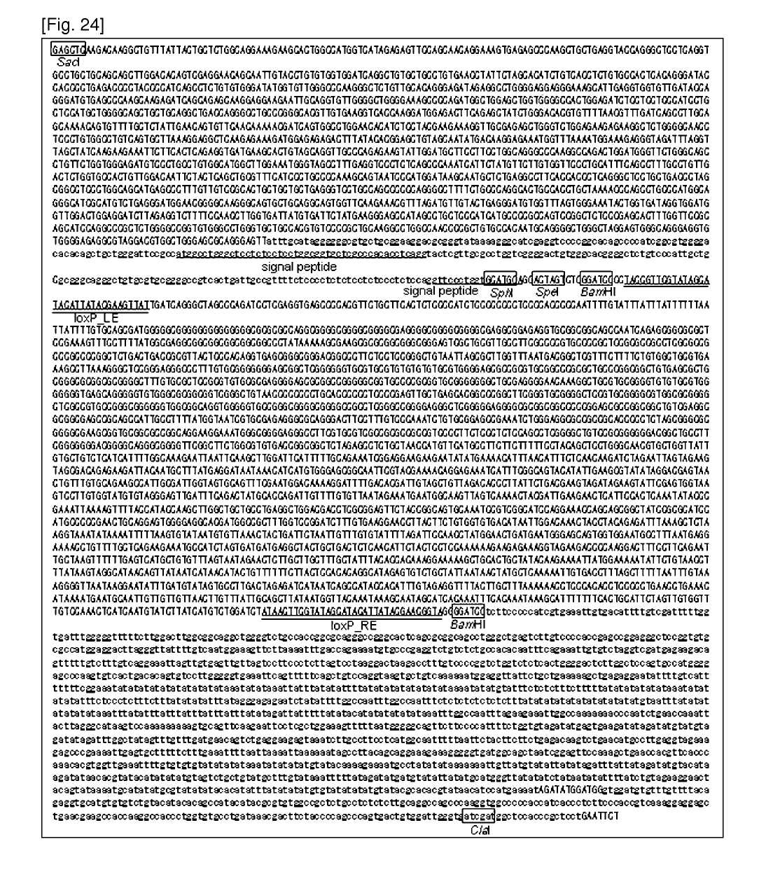

Furthermore, an example of the nucleotide sequence of a gene targeting vector of the present invention that targets a light-chain variable region is shown in SEQ ID NO: 20 (FIG. 24). Specifically, the present invention provides targeting vectors comprising a DNA construct comprising the nucleotide sequence of SEQ ID NO: 20 (in particular, the nucleotide sequence of positions 1 to 6861).

In FIG. 24 (SEQ ID NO: 20),

the nucleotides of positions 1 to 1868 correspond to a DNA sequence homologous to a DNA on the 5′ side of a DNA encoding an antibody variable region of an antibody-producing cell (a sequence containing a promoter, down to immediately before the start codon of the DNA encoding an antibody variable region);

the nucleotides of positions 1869 to 1914 correspond to a DNA encoding a signal peptide of the antibody variable region;

the nucleotides of positions 1915 to 2039 correspond to an intron DNA;

the nucleotides of positions 2040 to 2056 correspond to a DNA encoding the signal peptide of the antibody variable region;

the nucleotides of positions 2076 to 2109 correspond to the DNA sequence of loxP_LE;

the nucleotides of positions 2137 to 3478 correspond to a promoter sequence of a marker gene;

the nucleotides of positions 3522 to 3944 correspond to a DNA sequence of the marker gene;

the nucleotides of positions 3967 to 4953 correspond to a DNA sequence of a poly A addition sequence of the marker gene;

the nucleotides of positions 4960 to 4993 correspond to the DNA sequence of loxP_RE; and

the nucleotides of positions 5002 to 6861 correspond to a DNA sequence homologous to a DNA on the 3′ side of the DNA encoding the antibody variable region of the antibody-producing cell.

The nucleotides of positions 1915 to 2039 (intron DNA) are removed by splicing, and thus the nucleotides of positions 1869 to 1914 are linked to the nucleotides of positions 2040 to 2056 to form the signal peptide sequence.

In the above vector, the DNA encoding a desired amino acid sequence is inserted between the signal peptide-encoding DNA and the DNA sequence of loxP_LE.

There is no particular limitation on the methods for introducing targeting vectors of the present invention into cells. Those skilled in the art can select appropriate gene transfer methods depending on the antibody-producing cells selected. When the antibody-producing cells are, for example, mammalian cells, examples of the methods include, but are not limited to, for example, calcium phosphate precipitation methods, nuclear microinjection, protoplast fusion, DEAE-dextran methods, cell fusion, Lipofectamine (GIBCO BRL) methods, lipofection methods, methods using the FuGENE6 reagent (Boehringer-Mannheim), and electroporation.

Antibody-producing cells of the present invention may be of any cell type and derived from any animal species, as long as they can produce antibodies. Antibody-producing cells such as those derived from humans, mice, sheep, rats, rabbits, and chickens, and cell lines and mutant cell lines thereof can be used. The antibody-producing cells also include, but are not limited to, B cells, human Burkitt's lymphoma cell lines (Ramos, BL2, etc.), the mouse pre-B cell line 18-81, and the mouse premature B cell line WEHI-231. The antibody-producing cells preferably include chicken-derived B cells, for example, the DT40 and DT40-SW cell lines derived from chicken antibody-producing cells. The DT40 cell line is a cell line derived from B lymphoma, and is characterized by trisomy of chromosome 2 (Baba, T. W., Giroir, B. P. and Humphries, E. H., Virology 144: 139-151, 1985). Furthermore, one can use the wild-type DT40 line or the DT40-SW line established by the present inventors, a mutant line whose antibody mutation feature can be regulated (on and off) by reversibly switching on and off the expression of the AID gene which controls the mutation feature (the details are described in Kanayama, N., Todo, K., Reth, M., Ohmori, H. Biochem. Biophys. Res. Commn. 327:70-75 (2005) and JP-A (Kokai) 2006-109711).

The antibody-producing cells of the present invention also include cells whose antibody-producing ability is artificially conferred by gene manipulation. Such cells with artificially conferred antibody-producing ability are not necessarily capable of producing whole antibody molecules. The cells may be capable of producing an antibody fragment comprising an antibody variable region required for antigen binding. The cells may produce chimeric antibodies, humanized antibodies, or non-natural molecules capable of binding to an antigen, or fragments of the molecules.

Antibody-producing cells of the present invention may have the characteristics described below.

The present invention also relates to methods for selecting cells in which homologous recombination has occurred between a DNA construct and a region comprising a DNA encoding an antibody variable region of an antibody-producing cell, wherein the DNA construct comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct.

Homologous recombination can be achieved by the methods described above. Cells in which the recombination has occurred can be selected, for example, using marker gene expression as an index. Alternatively, the cells can be selected using the absence of endogenous antibody expression in the antibody-producing cells as an index. Alternatively, the cells can be selected by combining the two indices. However, the selection methods are not limited to the above examples.

Cells in which homologous recombination has occurred between the DNA construct and the region comprising the DNA encoding an antibody variable region of an antibody-producing cell become incapable of producing the endogenous antibody. Thus, cells in which homologous recombination has occurred can be selected using the presence or absence of the endogenous antibody on the cell surface as an indicator. In the present invention, the production of a polypeptide comprising a desired amino acid sequence is inhibited by the presence of the DNA of (3) above. More specifically, the production of a polypeptide comprising a desired amino acid sequence and the amino acid sequence of an antibody constant region is inhibited by the presence of the DNA of (3) above. For this reason, even when a signal peptide is attached to the polypeptide comprising a desired amino acid sequence to display the polypeptide on the cell surface, as long as the DNA of (3) above is present, a polypeptide comprising the polypeptide and an antibody constant region is not displayed on the cell surface. Thus, cells in which homologous recombination has occurred can be efficiently selected using an antibody against the antibody constant region to detect the presence or absence of an antibody molecule on the cell surface as an indicator. The cell selection using the presence or absence of an antibody molecule on the cell surface as an indicator can be performed, for example, by flow cytometry, magnetic beads onto which an antibody that specifically binds to the antibody produced by an antibody-producing cell is immobilized, or the like. However, the selection methods are not limited to these examples.

The DNA of (3) above preferably comprises a drug-resistance gene as the marker gene to place selection pressure on cell growth. Such drug-resistance genes include, but are not limited to, for example, antibiotic-resistance genes such as the neomycin phosphotransferase gene, the blasticidin S deaminase gene, the puromycin N-acetyltransferase gene, the histidinol dehydrogenase gene, the hygromycin B phosphotransferase gene, and the xanthine-guanine phosphoribosyltransferase gene.

The present invention also relates to methods for producing antibody-producing cells that produce a polypeptide. Antibody-producing cells that produce a polypeptide can be prepared by obtaining recombinant cells using a method described herein, and removing the DNA described below from the genomic DNA of the cells using the method described below. As necessary, the methods may comprise the step of selecting cells from which the DNA described below has been removed. Cells from which the DNA described below has been removed can be selected using the production of the polypeptide as an indicator.

Herein, the DNA to be removed is:

a DNA that inhibits the production of a polypeptide comprising a desired amino acid sequence, and which can be removed from the DNA construct; and more specifically,

a DNA that inhibits the production of a polypeptide comprising a desired amino acid sequence and is located between two site-specific recombinase recognition sequences oriented in the same direction, and which comprises a promoter DNA and a marker gene that function in the cell, and can be removed from the DNA construct by a site-specific recombinase.

The present invention also relates to methods for producing an antibody-producing cell that produce a polypeptide, which comprises the steps of:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;

(b) selecting a cell in which homologous recombination has occurred between the DNA construct and the region comprising the DNA encoding an antibody variable region of the antibody-producing cell; and

(c) removing the DNA located between two site-specific recombinase recognition sequences oriented in the same direction from the genomic DNA of the cell selected in step (b), by reacting a site-specific recombinase with the site-specific recombinase recognition sequences in the genome of the cell.

In the methods of the present invention for producing antibody-producing cells that produce a polypeptide, a site-specific recombinase is reacted with the site-specific recombinase recognition sequences in the genome of an antibody-producing cell. By this, the DNA located between two site-specific recombinase recognition sequences oriented in the same direction is removed from the genomic DNA of the antibody-producing cell.

The DNA located between two site-specific recombinase recognition sequences oriented in the same direction inhibits the transcription of both or either one of the DNA encoding a desired amino acid sequence and the DNA encoding an antibody constant region, thereby inhibiting the normal production of transcripts of the DNA encoding a polypeptide comprising the desired amino acid sequence. Alternatively, the DNA located between two site-specific recombinase recognition sequences oriented in the same direction inhibits the normal splicing of transcripts of the DNA encoding the polypeptide comprising a desired amino acid sequence, or translation or processing (for example, folding) of the translation product, thereby inhibiting the normal production of the polypeptide comprising a desired amino acid sequence. Thus, the production of the polypeptide comprising a desired amino acid sequence is restored by removing the DNA located between two site-specific recombinase recognition sequences oriented in the same direction. When a signal peptide is attached to the polypeptide comprising a desired amino acid sequence, the polypeptide is displayed on the cell surface or secreted to the outside of the cells. In this case, whether the DNA located between two site-specific recombinase recognition sequences oriented in the same direction has been removed is assessed based on the presence or absence of the polypeptide displayed on the cell surface. Furthermore, successful targeting in the region encoding the antibody variable region can be assessed by the presence of the polypeptide displayed on the cell surface.

In the methods of the present invention for producing antibody-producing cells that produce a polypeptide, when a signal peptide is attached to or originally present in the polypeptide, the peptide is displayed on the cell surface or secreted to the outside of the cells.

In the methods of the present invention, (i) a gene targeting vector comprising a DNA encoding the amino acid sequence of an antibody heavy-chain variable region as the desired amino acid sequence can be introduced into antibody-producing cells.

Furthermore, (ii) a gene targeting vector comprising a DNA encoding the amino acid sequence of an antibody light-chain variable region as the desired amino acid sequence can be introduced into antibody-producing cells.

In the methods of the present invention, when the vector of (i) is introduced into antibody-producing cells, the cells produce an antibody that comprises the endogenous antibody light chain and an antibody heavy chain that has the amino acid sequence of the endogenous antibody heavy-chain constant region and the amino acid sequence of the exogenous antibody heavy-chain variable region inserted into the vector.

Alternatively, when the vector of (ii) is introduced into antibody-producing cells, the cells produce an antibody that comprises the endogenous antibody heavy chain and an antibody light chain that has the amino acid sequence of the endogenous antibody light-chain constant region and the amino acid sequence of the exogenous antibody light-chain variable region inserted into the vector.

Alternatively, when the vectors of (i) and (ii) are introduced into antibody-producing cells, the cells produce an antibody that comprises an antibody heavy chain that has the amino acid sequence of the endogenous antibody heavy-chain constant region and the amino acid sequence of the exogenous antibody heavy chain variable region inserted into the vector of (i), and an antibody light chain that has the amino acid sequence of the endogenous antibody light-chain constant region and the amino acid sequence of the exogenous antibody light-chain variable region inserted into the vector of (ii).

The present invention relates to methods for producing antibody-producing cells that produce such antibodies.

The present invention also provides methods for producing an antibody-producing cell that produces a polypeptide into which a mutation is introduced, which comprise the steps of:

(a) allowing homologous recombination between a DNA construct and a region comprising a DNA encoding an antibody variable region of an antibody-producing cell expressing the AID gene, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence and which can be removed from the DNA construct;

(b) selecting a cell in which homologous recombination has occurred between the DNA construct and the region comprising the DNA encoding an antibody variable region of the antibody-producing cell; and

(c) removing the DNA of (3) from the genomic DNA of the cell selected in step (b).

As necessary, the methods of the present invention may further comprise the step of:

(d) selecting a cell from which the DNA of (3) has been removed. Cells from which the DNA of (3) has been removed can be selected using the production of the polypeptide as an indicator.

The targeting vectors of the present invention may comprise a DNA homologous to a DNA of the antibody gene locus, in addition to the DNAs of (1) to (3) above.

Furthermore, when the DNA encoding a desired amino acid sequence has no signal peptide-encoding DNA, a DNA encoding a signal peptide may be inserted at the 5′ side of the DNA encoding a desired polypeptide in the gene targeting vectors to enhance the cell-surface expression or secretion of the polypeptide encoded by the DNA.

The “polypeptide into which a mutation is introduced” can also be referred to as a “modified polypeptide”.

Mutations include substitutions, insertions, deletions, additions, and combinations thereof.

In the methods of the present invention, (i) a gene targeting vector comprising a DNA encoding the amino acid sequence of an antibody heavy-chain variable region as the desired amino acid sequence can be introduced into antibody-producing cells.

Furthermore, (ii) a gene targeting vector comprising a DNA encoding the amino acid sequence of an antibody light-chain variable region as the desired amino acid sequence can be introduced into antibody-producing cells.

In the methods of the present invention, when the vector of (i) is introduced into antibody-producing cells, the cells produce an antibody that comprises the endogenous antibody light chain and an antibody heavy chain that has the amino acid sequence of the endogenous antibody heavy-chain constant region and the amino acid sequence of the exogenous antibody heavy-chain variable region inserted into the vector.

Alternatively, when the vector of (ii) is introduced into antibody-producing cells, the cells produce an antibody that comprises the endogenous antibody heavy chain and an antibody light chain that has the amino acid sequence of the endogenous antibody light-chain constant region and the amino acid sequence of the exogenous antibody light-chain variable region inserted into the vector.

Alternatively, when the vectors of (i) and (ii) are introduced into antibody-producing cells, the cells produce an antibody that comprises an antibody heavy chain that has the amino acid sequence of the endogenous antibody heavy-chain constant region and the amino acid sequence of the exogenous antibody heavy-chain variable region inserted into the vector of (i), and an antibody light chain that has the amino acid sequence of the endogenous antibody light-chain constant region and the amino acid sequence of the exogenous antibody light-chain variable region inserted into the vector of (ii).

As a result of AID gene expression, mutations are introduced at a frequency not only into the DNA encoding the amino acid sequence of an endogenous antibody variable region but also into the DNA encoding the amino acid sequence of an antibody heavy-chain variable region inserted into the vector of (i) and the DNA encoding the amino acid sequence of an antibody light-chain variable region inserted into the vector of (ii). Thus, the cells produce antibodies in which mutations are introduced into a desired antibody variable region.

The present invention relates to methods for producing antibody-producing cells that produce the antibodies into which mutations are introduced.

In the above methods for producing an antibody-producing cell that produces a polypeptide into which a mutation is introduced, the DNA of (3) in step (a) can also be described as a DNA that inhibits the production of a polypeptide comprising a desired amino acid sequence and is located between two site-specific recombinase recognition sequences oriented in the same direction, and which comprises a promoter DNA and a marker gene that function in the cell, and can be removed from the DNA construct by a site-specific recombinase.

Furthermore, the step of (c) can also be described as removing the DNA located between two site-specific recombinase recognition sequences oriented in the same direction from the genomic DNA of the cell selected in step (b), by reacting a site-specific recombinase at the site-specific recombinase recognition sequences in the genome of the cell.

In the present invention, cells expressing the AID gene include cells that have the endogenous AID gene and can express the gene. More specifically, such cells include the chicken B cell line DT40, human Burkitt's lymphoma cell lines (Ramos, BL2, etc.), the mouse pre-B cell line 18-81, and the mouse premature B cell line WEHI-231, but are not limited thereto. When the cells express the endogenous AID gene, mutations are introduced into a DNA encoding a desired amino acid sequence inserted into the antibody variable region gene locus via recombination.

The present invention also provides methods for producing an antibody-producing cell that produces a polypeptide into which a mutation is introduced, which comprise the steps of:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and which can be removed from the DNA construct,

wherein the AID gene expression can be artificially switched on and off in the antibody-producing cell;

(b) selecting a cell in which homologous recombination has occurred between the DNA construct and the region comprising a DNA encoding an antibody variable region of the antibody-producing cell;

(c) removing the DNA of (3) from the genomic DNA of the cell selected in step (b);

(d) switching the AID gene expression on and off; and

(e) selecting a cell expressing the AID gene.

In the above methods for producing an antibody-producing cell that produces a polypeptide into which a mutation is introduced, step (b) can also be described as a step of selecting a cell that does not display the endogenous antibody on the cell surface, a step of selecting a cell that expresses the marker gene, or a step of selecting a cell that expresses the marker gene and does not display the endogenous antibody on the cell surface.

The present invention also relates to methods for producing an antibody-producing cell that produces a polypeptide into which a mutation is introduced, which comprise the steps of:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;

wherein the endogenous AID gene in the antibody-producing cell is functionally destroyed, and the antibody-producing cell comprises a DNA construct comprising

a promoter DNA that functions in the cell, and

a DNA that is located between two site-specific recombinase recognition sequences oriented in opposite directions, and which comprises an exogenous AID gene and can be inverted by a site-specific recombinase;

(b) selecting a cell in which homologous recombination has occurred between the DNA construct and the region comprising a DNA encoding an antibody variable region of the antibody-producing cell;

(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell;

(d) inverting the DNA located between the site-specific recombinase recognition sequences oriented in opposite directions, by reacting a site-specific recombinase at the site-specific recombinase recognition sequences in the genome of the cell selected in step (b), to switch the AID gene expression on and off; and

(e) selecting a cell expressing the AID gene.

The present invention also relates to methods for producing an antibody-producing cell that produces a polypeptide into which a mutation is introduced, which comprise the steps of:

(a) allowing homologous recombination between a DNA construct and a region comprising a DNA encoding an antibody variable region of an antibody-producing cell, by introducing into the antibody-producing cell a targeting vector comprising a DNA construct that comprises:

(1) a promoter DNA that functions in the cell;

(2) a DNA that encodes a desired amino acid sequence; and

(3) a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct,

wherein the endogenous AID gene in the antibody-producing cell is functionally destroyed, and the antibody-producing cell comprises

a DNA construct comprising

a promoter DNA that functions in the cell; and

a DNA that is located between two site-specific recombinase recognition sequences oriented in opposite directions, and which can be inverted by a site-specific recombinase, and that comprises an exogenous AID gene; and

a DNA construct comprising a promoter DNA that functions in the cell and a DNA encoding the site-specific recombinase,

wherein in the antibody-producing cell, the site-specific recombinase is activated in the presence of an extracellular stimulus, and is not activated in the absence of the extracellular stimulus;

(b) selecting a cell in which homologous recombination has occurred between the DNA construct and the region comprising a DNA encoding an antibody variable region of the antibody-producing cell;

(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell via activation of the site-specific recombinase activity by applying an extracellular stimulus to the cell;

(d) switching the AID gene expression on and off by inverting the DNA located between the two site-specific recombinase recognition sequences oriented in opposite directions, by reacting a site-specific recombinase at the site-specific recombinase recognition sequences in the genome of the cell selected in step (b) via activation of the site-specific recombinase activity by applying an extracellular stimulus to the cell; and

(e) selecting a cell expressing the AID gene.

In the methods of the present invention for producing antibody-producing cells that produce a polypeptide into which a mutation is introduced, when a signal peptide is attached to or originally present in the polypeptide, the peptide is displayed on the cell surface or secreted to the outside of the cell.

In antibody-producing cells of the present invention, the presence or absence of AID gene expression can be regulated (the AID gene expression can be switched on and off) by reacting a site-specific recombinase at the site-specific recombinase recognition sequences in the genome of the cells. Thus, one can start or stop introducing mutations in the DNA encoding a desired amino acid sequence. When the AID gene expression is switched on, the mutation-introducing function becomes active and the mutagenesis occurs in a sustained manner in the DNA encoding a desired amino acid sequence. On the other hand, the DNA mutagenesis is kept blocked when the AID gene expression is switched off. That is, once the expression is switched off, the mutated DNA can not be further mutated after the switch-off, and thus the mutations are maintained.

Sometimes it takes time for the mutation to be introduced in to a DNA after initiation of AID gene expression. If the DNA mutagenesis does not start immediately after initiation of AID gene expression, it is preferable to culture the cells for a certain period (for example, for one month). The culture conditions may be “40 degrees C. in the presence of 5% CO₂”, but are not limited thereto.

By switching the AID gene expression on/off as a mechanism for regulating gene mutation, mutations can be introduced into target DNAs encoding a desired amino acid sequence. Alternatively, one can prevent the occurrence of further mutation events in a DNA after a certain number of mutations have been introduced.

By reacting a site-specific recombinase with antibody-producing cells, the DNA located between site-specific recombinase recognition sequences is removed, and thus the production of a polypeptide comprising a desired amino acid sequence is restored. By selecting cells in which this has occurred, one can obtain antibody-producing cells that produce a polypeptide comprising a desired amino acid sequence. Furthermore, by selecting such cells, one can obtain the polypeptide comprising a desired amino acid sequence and a DNA encoding the polypeptide. As necessary, cells reacted with a site-specific recombinase may be cultured for a certain period until the cells produce the polypeptide into which mutations are introduced.

Meanwhile, the antibody-producing cells of the present invention that express the AID gene may be cells in which the presence or absence of AID gene expression can be artificially regulated. Such cells include, but are not limited to, cells prepared by introducing an exogenous AID gene into antibody-producing cells that do not have the endogenous AID gene, and cells prepared by introducing an exogenous AID gene into antibody-producing cells that have an inactivated endogenous AID gene. The cells into which an exogenous AID gene is introduced can be obtained by introducing into antibody-producing cells a vector comprising a DNA in which the AID gene is operably linked to a promoter that functions in the cells. Those skilled in the art can think of various promoters and select an appropriate promoter. Such promoters include, but are not limited to, for example, the beta-actin promoter, immunoglobulin promoter, cytomegalovirus promoter, CAG promoter, and EF1alpha promoter.

Furthermore, the antibody-producing cells of the present invention include cells whose endogenous AID gene is functionally destroyed, and which comprise a DNA construct comprising

a promoter DNA that functions in the cells; and

a DNA that comprises an exogenous AID gene and is located between two site-specific recombinase recognition sequences oriented in the opposite directions,

wherein the DNA located between the site-specific recombinase recognition sequences can be inverted by a site-specific recombinase. Herein, the DNA construct is sometimes referred to as “exogenous AID gene construct”.

“Gene that is functionally destroyed” can also be referred to as “gene that is inactivated”, “gene that is deleted”, or “gene that is knocked out”. Gene inactivation includes both complete and partial suppression of gene expression.

Herein, gene inactivation means that gene expression is suppressed by partial deletion, substitution, insertion, addition, or the like in the nucleotide sequence of a gene. The “suppression of gene expression” also includes the case where a gene is expressed but the produced protein does not have the normal function.

In the present invention, heterozygous knockout of the AID gene can be achieved since only one of the two alleles is inactivated (JP 2006-109711; Biochem. Biophys. Res. Commun. 327:70 (2005)). AID gene knockout can be achieved, for example, by homologous recombination using a gene targeting vector. Homologous recombination is a method for modifying a gene of interest by homologously recombining a gene in the chromosome and a foreign DNA. The gene targeting vectors may carry a selection marker as an insert to identify cells in which homologous recombination has occurred. Such selection marker genes include, but are not limited to, antibiotic-resistance genes such as the neomycin phosphotransferase gene, blasticidin S deaminase gene, puromycin N-acetyltransferase gene, histidinol dehydrogenase gene, hygromycin B phosphotransferase gene, and xanthine-guanine phosphoribosyltransferase gene; fluorescent protein genes such as GFP and DsRed; and genes of chromogenic enzymes such as beta-galactosidase (lacZ) and beta-lactamase. Poly A may be attached to the 3′ end of the marker genes.

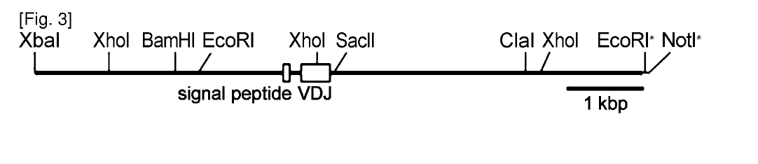

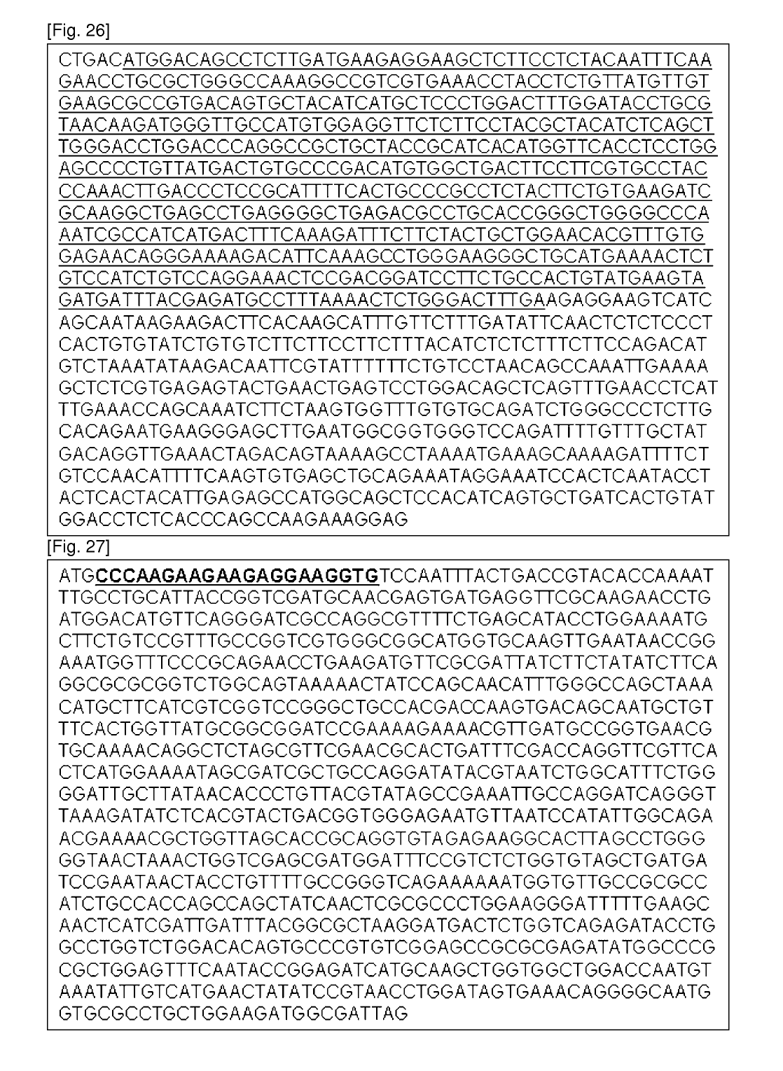

The exogenous AID gene construct of the present invention is preferably integrated into the genome of antibody-producing cells. There is no particular limitation on the integration position of the exogenous AID gene construct in the genome. However, the construct may be integrated, for example, into a position within one of the alleles of the endogenous gene locus (JP 2006-109711; Biochem. Biophys. Res. Commun. 327:70 (2005)). In this case, for example, based on the information in FIG. 3 of JP 2006-109711, an AID gene targeting vector is constructed to comprise a DNA construct comprising a DNA in which a promoter DNA that functions in the antibody-producing cells is operably linked to an exogenous AID gene (for example, SEQ ID NO: 50 (FIG. 26), GeneBank accession NO. XM_{—}416483) located between two site-specific recombinase recognition sequences oriented in opposite directions. This vector is introduced into the cells. As a result, homologous recombination occurs between the above DNA construct and the endogenous AID gene of the antibody-producing cells. Thus, antibody-producing cells that have the exogenous AID gene construct integrated into the genome can be obtained.

The promoters include, but are not limited to, those described above.

The exogenous AID gene located between site-specific recombinase recognition sequences can be inverted by reaction of a site-specific recombinase. When the exogenous AID gene is oriented in the same direction as the promoter DNA, the AID gene is expressed because the promoter DNA is operably linked to the exogenous AID gene. Meanwhile, when the exogenous AID gene is oriented in the opposite direction relative to the promoter DNA, the AID gene is not expressed.

If the endogenous AID gene is inactive but an exogenous AID gene construct is present in cells, and a site-specific recombinase is reacted with the cells, the recombinase recognizes site-specific recombinase recognition sequences in the cells, and inverts the region located between the two site-specific recombinase recognition sequences (i.e., the orientation of the AID gene relative to the promoter is altered from opposite direction to the same direction, or the same direction to opposite direction).

Thus, when the orientation of the AID gene relative to the promoter is altered from opposite direction to the same direction, the AID gene expression is switched from off to on. Conversely, when the orientation of the AID gene relative to the promoter is altered from the same direction to opposite direction, the AID gene expression is switched from on to off.

More specifically, when cells contain an exogenous AID gene construct, and the promoter and the AID gene are oriented in the same direction in the construct, the orientation of the exogenous AID gene is reverted to the opposite direction relative to the promoter in the construct by reaction of a site-specific recombinase with the cells. This terminates the AID gene expression, and thus stops the introduction of mutations into the polypeptide expressed from the DNA encoding a desired amino acid sequence that has been integrated into the genome of the antibody-producing cells.

If the orientation of the exogenous AID gene of the exogenous AID gene construct is not inverted even when a site-specific recombinase is reacted with the cells, the AID gene continues to be expressed by the promoter, since the AID gene is oriented in the same direction as the promoter of the gene.

According to the present invention, antibody-producing cells that produce a polypeptide comprising a desired amino acid sequence can be obtained by selecting cells in which the promoter and the AID gene are oriented in the same direction after reacting a site-specific recombinase with the cells. The present invention also allows production of a polypeptide comprising a desired amino acid sequence and a DNA encoding the polypeptide.

Alternatively, when cells contain an exogenous AID gene construct, and the promoter and the AID gene are oriented in the opposite directions in the construct, the orientation of the exogenous AID gene is reverted to the same direction as the promoter in the construct by reacting a site-specific recombinase with the cells. This starts the AID gene expression, and mutations are introduced into the polypeptide expressed from the DNA encoding a desired amino acid sequence that has been integrated into the genome of the antibody-producing cells.

If the orientation of the exogenous AID gene of the exogenous AID gene construct is not inverted even when a site-specific recombinase is reacted with the cells, the orientation of the AID gene remains in the opposite direction relative to the promoter of the gene, and the promoter-mediated AID gene expression remains terminated.

According to the present invention, antibody-producing cells that produce a modified polypeptide can be obtained by selecting cells in which the promoter and the AID gene are oriented in the same direction after reaction of a site-specific recombinase with the cells. The present invention also allows production of DNAs encoding modified polypeptides.

It is advantageous to design an exogenous AID gene construct to contain two marker genes, of which one is in the same orientation as the AID gene and the other is inserted in the opposite orientation relative to the AID gene, because it allows selection using one of the marker genes, regardless of whether the AID gene is placed in the same or opposite orientation relative to the promoter. Such constructs include exogenous AID gene constructs having the following characteristics:

an exogenous AID gene construct comprising a DNA sequence that contains the following sequences from the 5′ to 3′ end:

a first site-specific recombinase recognition sequence,

a first marker gene in the forward orientation,

a second marker gene in the reverse orientation,

an AID gene in the reverse orientation, and

a second site-specific recombinase recognition sequence.

It is necessary to design the construct to have either marker gene to be expressed only when it is oriented in the same direction as the promoter. For example, when the GFP gene is used as a second marker gene in the above construct, an IRES sequence is preferably inserted between the second marker gene and the AID gene to obtain highlevel expression of the GFP gene. For example, one can use DNA constructs that contain the following sequences from the 5′ to 3′ end:

a promoter in the forward orientation,

a first site-specific recombinase recognition sequence,

a first marker gene in the forward orientation,

a poly A addition sequence in the forward orientation,

a poly A addition sequence in the reverse orientation,

a second marker gene in the reverse orientation,

an IRES sequence in the reverse orientation,

an AID gene in the reverse orientation, and

a second site-specific recombinase recognition sequence that is oriented in the opposite direction relative to the first site-specific recombinase recognition sequence.

In an alternative embodiment, it is possible to use cells introduced with a DNA construct in which the AID gene is located between two site-specific recombinase recognition sequences oriented in the same direction. In this case, the AID gene is excised out by the action of a site-specific recombinase, and thus the cells become irreversibly AID-deficient. In order to again switch on the AID gene expression in the cells, the AID gene needs to be re-introduced into the cells, and this method may be used because it can completely shut off of the AID gene at 100% efficiency. The exogenous AID gene constructs may have various types of marker genes to efficiently select clones in which the AID gene is oriented in a particular direction. Such selection marker genes include, but are not limited to, antibiotic-resistance genes such as the neomycin phosphotransferase gene, blasticidin S deaminase gene, puromycin N-acetyltransferase gene, histidinol dehydrogenase gene, hygromycin B phosphotransferase gene, and xanthine-guanine phosphoribosyltransferase gene; fluorescent protein genes such as GFP and DsRed; and genes of chromogenic enzymes such as beta-galactosidase (lacZ) and beta-lactamase.

When a promoter that functions in antibody-producing cells and the AID gene are oriented in the same direction in an exogenous AID gene construct, and thus the AID gene is placed in an expressible manner, the DNA construct is designed to place the marker gene in the same orientation as the AID gene so that the marker gene can also be expressed. In this case, antibody-producing cells expressing the marker gene can also express the AID gene. Thus, cells expressing the AID gene can be selected using the expression of the marker gene as an indicator (based on the phenotype).

On the other hand, when a promoter that functions in antibody-producing cells is oriented in the opposite direction relative to the AID gene in an exogenous AID gene construct, and thus the AID gene is in an inexpressible state, the DNA construct is designed to place the marker gene in the opposite orientation relative to the AID gene (to place the marker gene in the same orientation as the promoter) so that the marker gene can be expressed. In this case, antibody-producing cells expressing the marker gene do not express the AID gene. Thus, cells without AID gene expression can be selected using the expression of the marker gene as an index (based on the phenotype).

In the present invention, cells that produce a polypeptide into which mutations are introduced can be selected whether the AID gene expression in an ON or OFF state. A polypeptide into which desired mutations are introduced or a DNA encoding the polypeptide is isolated from isolated cells, and they are tested to see whether they are the desired polypeptide or DNA encoding the polypeptide. If an isolated polypeptide or DNA is not the polypeptide into which a desired mutation is introduced or a DNA encoding the polypeptide, and the AID gene expression in the cells is in an ON state, it is possible to further culture the cells. As the cells are cultured for a longer period, more mutations are accumulated in the polypeptide. Thus, it is possible to isolate a polypeptide or a DNA encoding the polypeptide from the further cultured cells, and test whether it is the polypeptide into which desired mutations are introduced or a DNA encoding the polypeptide.

On the other hand, if the isolated polypeptide or DNA is not the polypeptide into which desired mutations are introduced or a DNA encoding the polypeptide, and the cellular AID gene expression in an OFF state, then the expression of the AID gene is switched on in the cells. This leads the cells to produce polypeptides into which mutations are further introduced. It is possible to isolate a polypeptide into which mutations are introduced or DNA encoding the polypeptide from the cells, and test whether it is the polypeptide into which desired mutations are introduced or a DNA encoding the polypeptide.

An example of the methods of the present invention is described below. However, the methods are not limited to this example.

(1) For example, a gene targeting vector of the present invention is introduced into antibody-producing cells that do not express the AID gene (antibody-producing cells in which the AID gene expression is switched off). Cells into which the vector has been successfully introduced do not express an endogenous antibody molecule. Even if a gene targeting vector of the present invention is introduced into antibody-producing cells that express the AID gene (antibody-producing cells in which AID gene expression is switched on), cells into which the vector has been successfully introduced do not express an endogenous antibody molecule.

(2) Cells that do not express the endogenous antibody molecule are selected.

(3) Then, a site-specific recombinase is reacted with site-specific recombinase recognition sequences in the genome of the cells into which the vector is introduced. The DNA located between the site-specific recombinase recognition sequences is removed by the recombinase, and thus the cells express a polypeptide (a chimeric protein comprising a desired amino acid sequence and an antibody constant region amino acid sequence). Furthermore, the site-specific recombinase that reacted with the cells inverts the orientation of the AID gene in the exogenous AID gene construct. The inversion of the AID gene results in expression of the gene (the AID gene expression is switched on). However, the inversion of the AID gene may not occur in some cases. This results in a mixture of cells in which the AID gene expression is switched on and off.

(4) Next, cells that produce the polypeptide (a chimeric protein comprising a desired amino acid sequence and an antibody constant-region amino acid sequence) and express the AID gene are selected. Alternatively, cells that produce the polypeptide (a chimeric protein comprising a desired amino acid sequence and an antibody constant-region amino acid sequence) but do not express the AID gene are selected. When the latter cells are selected, the site-specific recombinase is further reacted with the cells. Cells in which the AID gene expression is switched on by the treatment are selected.

(5) Then, cells that produce the polypeptide having a desired property are isolated from the selected cells expressing the AID gene and the polypeptide (a chimeric protein comprising a desired amino acid sequence and an antibody constant-region amino acid sequence). Alternatively, the selected cells are cultured for a certain period, and then cells that produce the polypeptide having a desired property are selected from the cultured cells. The AID gene expression may be switched off by reacting a site-specific recombinase with the cells before or after isolating the cells.

(6) When cells that produce the polypeptide having a desired property (a chimeric protein comprising a desired amino acid sequence and an antibody constant-region amino acid sequence) are not obtained, the step of (5) may be repeated after culturing the cells for a certain period. If the AID gene expression is switched off in (5), the expression is switched on again before culturing.

(7) a DNA can be isolated from isolated cells to confirm the presence of mutations introduced into the DNA.

A polypeptide into which mutations are introduced or a DNA encoding the polypeptide can be isolated using the methods described below. Whether a polypeptide into which mutations are introduced or a DNA encoding the polypeptide is of interest can be assessed by determining the activity of the polypeptide into which mutations are introduced or the nucleotide sequence of the polypeptide-encoding DNA. Alternatively, when a polypeptide into which mutations are introduced is an antibody, cytokine receptor, or such, the activity (specificity, affinity, etc.) of binding to an antigen or ligand can be used as an indicator, or when the polypeptide is an enzyme or such, an enzymatic activity can be used as an indicator, to test whether the polypeptide into which mutations are introduced or a DNA encoding the polypeptide is of interest.

Using the above methods, clones that produce a polypeptide into which desired mutations are introduced can be isolated from a prepared cell population. Furthermore, clones that produce a polypeptide into which more desired mutations are introduced can be obtained by switching on the AID gene expression and repeating mutagenesis in the obtained clones.

Cells that produce a polypeptide having a desired property can be selected by screening using the presence or absence of the desired property in a polypeptide produced as an indicator. Such screening can be carried out after isolating polypeptides from cells, or using the cells without polypeptide isolation. Herein, a DNA encoding a signal peptide may be inserted between a DNA that functions in the antibody-producing cells and a DNA encoding a desired amino acid sequence, so that the polypeptide is displayed on the cell surface or secreted to the outside of the cells. When the polypeptide is secreted to the outside of the cells, screening may be performed using culture supernatants. After selecting cells as described above, DNAs may be isolated from the cells to determine mutations introduced into the nucleotide sequences.

In the present invention, a site-specific recombinase can be reacted with cells, for example, by the methods described below. However, the methods are not limited to these examples.

(1) Tetracycline, doxycycline, or the like is added to cells containing a DNA construct in which a DNA encoding a site-specific recombinase attached to (or containing) a nuclear translocation signal is placed on the 3′ side of a promoter regulated by tetracycline, doxycycline, etc.
Gossen, M. Bujard, H. (1992) Tight control of gene expression in mammalian cells by tetracycline responsive promoters. Proc. Natl. Acad. Sci. USA 89:5547-5551.
Gossen, M., Freundlieb, S., Bender, G., Muller, G., Hillen, W. Bujard, H. (1995) Transcriptional activation by tetracycline in mammalian cells. Science 268:1766-1769.
Urlinger, S., Baron, U., Thellmann, M., Hasan, M. T., Bujard, H. Hillen, W. (2000) Exploring the sequence space for tetracycline-dependent transcriptional activators: Novel mutations yield expanded range and sensitivity. Proc. Natl. Acad. Sci. USA 97(14):7963-7968.

(2) A vector for expressing the site-specific recombinase is transiently transfected into the cells.

(3) The site-specific recombinase attached to (or containing) a nuclear translocation signal is introduced into the cells.

(4) An extracellular stimulus (for example, 4-hydroxytamoxifen) is applied to cells containing a DNA construct comprising a DNA encoding a site-specific recombinase that is activated in the presence of the extracellular stimulus and not in the absence of the stimulus.

Antibody-producing cells of the present invention may contain a DNA construct comprising a DNA in which a promoter DNA that functions in the cells is operably linked to a DNA encoding a site-specific recombinase. Hereinbelow, this DNA construct is sometimes referred to as “a site-specific recombinase gene construct”.

As described above, the promoter that functions in antibody-producing cells includes, but is not limited to, the beta-actin promoter, immunoglobulin promoter, cytomegalovirus promoter, CAG promoter, and EF1alpha promoter.

The site-specific recombinase may be in a form that is activated in the presence of an extracellular stimulus but not in the absence of the stimulus. Such site-specific recombinases include, but are not limited to, a fusion protein of a site-specific recombinase with an estrogen receptor or a polypeptide comprising an estrogen-binding domain thereof.

In the site-specific recombinase gene construct of the present invention, a nuclear translocation signal may be inserted at the N terminus of the site-specific recombinase. Furthermore, it is preferred that a poly A addition sequence is placed at the 3′ end of a DNA encoding the site-specific recombinase or the fusion protein of a site-specific recombinase with a polypeptide comprising an estrogen-binding domain.

Meanwhile, the site-specific recombinase gene constructs of the present invention may comprise selection marker genes. Such selection marker genes include, but are not limited to, antibiotic-resistance genes such as the neomycin phosphotransferase gene, blasticidin S deaminase gene, puromycin N-acetyltransferase gene, histidinol dehydrogenase gene, hygromycin B phosphotransferase gene, and xanthine-guanine phosphoribosyltransferase gene; fluorescent protein genes such as GFP and DsRed; and genes of chromogenic enzymes such as beta-galactosidase (lacZ) and beta-lactamase. Poly A may be attached to the 3′ end of the marker genes.

There is no limitation on the estrogen receptors of the present invention as long as they have ligand-binding activity. The estrogen receptors include both wild-type (for example, SEQ ID NOs: 52 and 53) and mutant estrogen receptors. Mutant estrogen receptors include those that respond to only 4-hydroxytamoxifen but not to estradiol which is the authentic ligand. More specifically, mutant estrogen receptors include, but are not limited to, mutant mouse estrogen receptors comprising an amino acid substitution of glycine with arginine at position 525.

When estrogen serves as a sex hormone in the species from which the antibody-producing cells are derived, or when the antibody-producing cells are responsive to estrogen, it is preferable to use a mutant estrogen receptor with impaired estrogen-binding activity. When the receptor is used in a form of fusion protein, its ligand-binding domain alone may be fused with a site-specific recombinase.

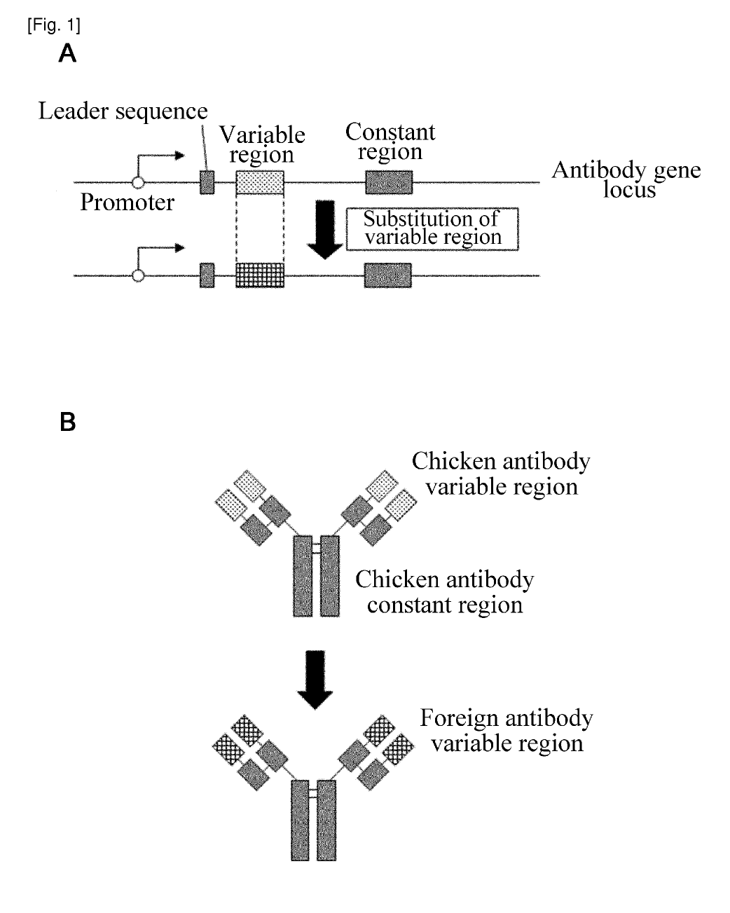

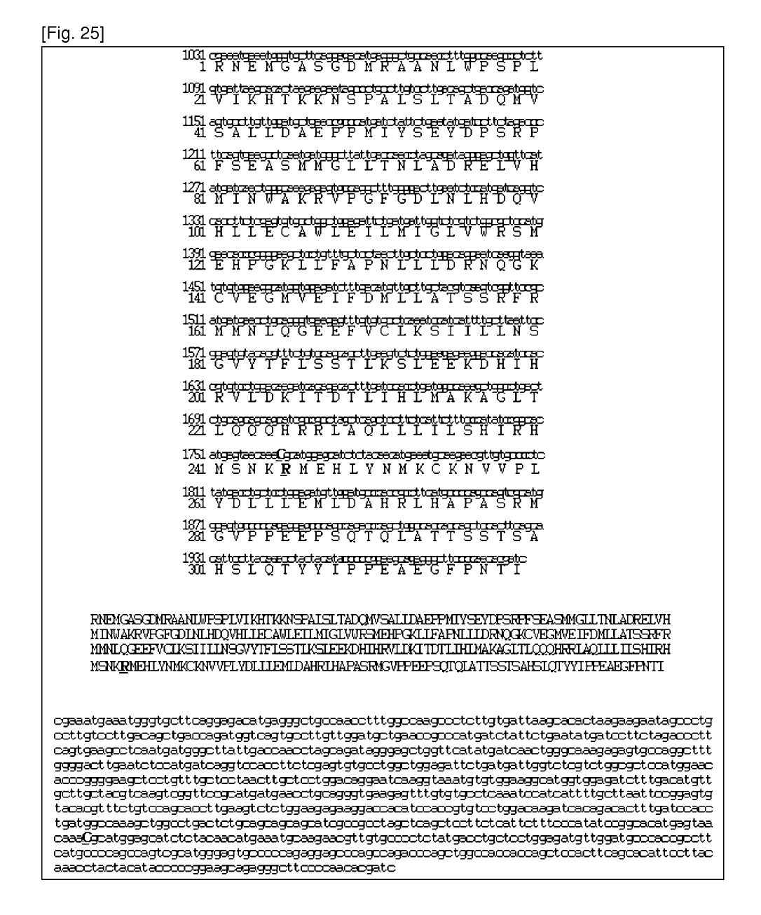

Those skilled in the art can prepare mutant estrogen receptors, for example, according to the method described in document (1) shown below (this document describes preparation of multiple types of mutants). Meanwhile, document (2) reports that a mutant estrogen receptor comprising a substitution of glycine with arginine at position 525 in its amino acid sequence responds to only 4-hydroxytamoxifen but not to estradiol which is the authentic ligand. Furthermore, documents (3) and (4) disclose that the tamoxifen-binding domain of a mutant estrogen receptor is used in a form of fusion protein. Those skilled in the art can prepare site-specific recombinase gene constructs by appropriately referring to these documents. In the Examples described herein, a Cre recombinase (for example, SEQ ID NO: 51 (FIG. 27)) fused with the tamoxifen-binding domain (SEQ ID NOs: 40 and 41 (FIG. 25)) of a mutant estrogen receptor is used. The mutant estrogen receptor used in the Examples is described in document (4) (for example, FIG. 1).
(1) Fawell et al. Characterization and colocalization of steroid binding and dimerization activities in the mouse estrogen receptor. Cell (1990) vol. 60 (6) pp. 95-362
(2) Danielian et al. Identification of residues in the estrogen receptor that confer differential sensitivity to estrogen and hydroxytamoxifen. Mol Endocrinol (1993) vol. 7 (2) pp. 232-40
(3) Littlewood et al. A modified oestrogen receptor ligand-binding domain as an improved switch for the regulation of heterologous proteins. Nucleic Acids Res (1995) vol. 23 (10) pp. 1686
(4) Zhang et al. Inducible site-directed recombination in mouse embryonic stem cells. Nucleic Acids Res (1996) vol. 24 (4) pp. 543-8

The site-specific recombinase gene constructs of the present invention include, but are not limited to, constructs having the structures described below (those corresponding to the constructs illustrated in FIG. 1 of document (4) above).

A construct comprising the DNAs of (1) and (2) below, in which the DNA of (1) is operably linked to the DNA of (2) (a construct named “pANCreMer” in FIG. 1a of document (4) above):

(1) a promoter DNA that functions in an antibody-producing cell;

(2) a DNA in which the DNAs of (i) and (ii) below are linked in-frame;

(i) a DNA encoding a site-specific recombinase, which comprises a DNA fragment encoding a nuclear translocation signal immediately after the start codon; and

(ii) a DNA encoding an estrogen receptor or a polypeptide comprising an estrogen-binding domain thereof.

A construct comprising the DNAs of (1) and (2) below, in which the DNA of (1) is operably linked to the DNA of (2) (a construct named “pANMerCreMer” in FIG. 1b of document (4) above):

(1) a promoter DNA that functions in an antibody-producing cell;

(2) a DNA in which the DNAs of (i) and (ii) below are linked in-frame in the order of (ii)-(i)-(ii) from the 5′ to 3′ side of the vector DNA chain:

(i) a DNA encoding a site-specific recombinase, which comprises a DNA fragment encoding a nuclear translocation signal immediately after the start codon; and

(ii) a DNA encoding an estrogen receptor or a polypeptide comprising an estrogen-binding domain thereof.

It is preferred that the DNA encoding a site-specific recombinase comprises a poly A addition sequence at its 3′ end.

Cells containing such a site-specific recombinase gene construct can be obtained by introducing a prepared site-specific recombinase gene construct into antibody-producing cells using methods known to those skilled in the art. Preferably, the introduced site-specific recombinase gene construct is integrated into the genome.

The site-specific recombinase is activated in the presence of an extracellular stimulus but not in the absence of the stimulus.

Herein, “activated in the presence of an extracellular stimulus” means that by the stimulus, the site-specific recombinase is expressed in an active form capable of translocating into the nucleus, or the expressed site-specific recombinase translocates into the nucleus. In other words, it means that the site-specific recombinase becomes in a state capable of reacting with and specifically cleaving site-specific recombinase recognition sequences in the genome of the cells. Thus, the site-specific recombinase may be in any form as long as a DNA encoding the site-specific recombinase is expressed or the site-specific recombinase is activated in response to the stimulus. Those skilled in the art can think of various regulatory systems for stimulus-responsive expression or activation, and select an appropriate system from them. The site-specific recombinase constructs include, for example, a DNA construct in which a gene encoding an estrogen receptor or a polypeptide comprising an estrogen-binding domain thereof is linked in-frame with a site-specific recombinase cDNA, so that the site-specific recombinase can be expressed as a fusion protein with an estrogen receptor or a polypeptide comprising an estrogen-binding domain thereof (for details, see the Examples of JP-A (Kokai) 2006-109711). In this case, the site-specific recombinase is activated when an extracellular stimulus is applied to the cells. Thus, only when the stimulus is applied, the site-specific recombinase is activated (which means that the above fusion protein translocates into the nucleus and thus the site-specific recombinase can react with site-specific recombinase recognition sequences), and thus inverts the region that contains the AID gene and is located in between the site-specific recombinase recognition sequences. At the same time, the DNA located between two site-specific recombinase recognition sequences oriented in the same direction, and which contains a promoter DNA and a marker gene that function in the cells is removed.

The extracellular stimuli include, but are not limited to, stimuli for an estrogen receptor or estrogen-binding domain thereof. The “stimuli for an estrogen receptor or estrogen-binding domain thereof” include, but are not limited to, stimuli with estrogen (for example, estradiol) and derivatives thereof (for example, 4-hydroxytamoxifen which is an estrogen antagonist).

The antibody-producing cells of the present invention may have the characteristics of (a) and (b) below.

(a) Only one of the two alleles of the XRCC3 gene is inactivated in the antibody-producing cells.

(b) The frequency of introduction of point mutation is elevated as compared to cells having both alleles of the XRCC3 gene.

The AID gene-mediated mutagenesis of DNAs encoding a protein derived from antibody-producing cells occurs mainly by the mechanism called gene conversion. Point mutations due to single nucleotide substitution also occur but at a low frequency. Meanwhile, a method has been reported for converting the mode of mutation from gene conversion to point mutation in DT40 cells which are predominant in gene conversion mutation. It is known that point mutation can be induced in cells by inactivating only one of the two alleles of the XRCC3 (X-ray repair complementing defective repair in Chinese hamster cells 3) gene (JP-A (Kokai) 2009-60850). Thus, the present invention also includes antibody-producing cells in which only one of the two alleles of the XRCC3 gene is inactivated.

XRCC3 is a gene involved in the maintenance of chromosomal stability and repair of damaged DNA, and participates in homologous recombination in cells. The chicken XRCC3 gene has eight exons. The nucleotide sequence of the chicken XRCC3 gene is shown in SEQ ID NO: 39. The positions of the eight exons in SEQ ID NO: 39 are shown below:

exon 1, positions 1 to 215;

exon 2, positions 216 to 284;

exon 3, positions 285 to 422;

exon 4, positions 423 to 635;

exon 5, positions 636 to 790;

exon 6, positions 791 to 1003;

exon 7, positions 1004 to 1050; and

exon 8, positions 1051 to 1935.

In the antibody-producing cells of the present invention, only one of the two alleles of the XRCC3 gene which are present in the homologous chromosomes of the cells may be inactivated. The gene inactivation is described herein above. Gene-inactivated cells can be prepared, for example, by methods known to those skilled in the art, such as a method of homologous recombination using a targeting vector as described above.

In the present invention, it is possible to split any of the eight exons of the chicken XRCC3 gene. Multiple exons may be split. The sixth exon is preferably split.

Antibody-producing cells used in the present invention include, but are not limited to, B cells, human Burkitt's lymphoma cell lines (Ramos, BL2, etc.), the mouse pre-B cell line 18-81, and the mouse premature B cell line WEHI-231. The antibody-producing cells preferably include chicken-derived B cells such as DT40 and DT40-SW cell lines derived from chicken antibody-producing cells.

The present invention also relates to methods for producing a DNA encoding a polypeptide or a polypeptide into which a mutation is introduced, which comprise the steps of:

(a) producing an antibody-producing cell that produces a polypeptide or a polypeptide into which a mutation is introduced, by a method for producing an antibody-producing cell described herein; and (b) isolating a DNA encoding the polypeptide or the polypeptide into which a mutation is introduced from the antibody-producing cell of step (a).

DNAs can be isolated by methods known to those skilled in the art.

The antibody-producing cells of the present invention that produce a polypeptide or a polypeptide into which a mutation is introduced include:

antibody-producing cells that display a polypeptide or a polypeptide into which a mutation is introduced on the cell surface;

antibody-producing cells that secrete a polypeptide or a polypeptide into which a mutation is introduced to the outside of the cells; and

antibody-producing cells containing a polypeptide or a polypeptide into which a mutation is introduced in the cytoplasm.

The present invention also relates to methods for producing a polypeptide or a polypeptide into which a mutation is introduced, which comprise the steps of:

(a) producing an antibody-producing cell that produces a polypeptide or a polypeptide into which a mutation is introduced, by a method for producing an antibody-producing cell described herein; and

(b) isolating a polypeptide or a polypeptide into which a mutation is introduced from the antibody-producing cell of step (a) or a secretion material thereof.

When the antibody-producing cells display a polypeptide or a polypeptide into which a mutation is introduced on the cell surface, the polypeptide can be isolated by affinity chromatography after solubilization of the membrane fraction.

Alternatively, when the antibody-producing cells secrete a polypeptide or a polypeptide into which a mutation is introduced to the outside of the cells, the polypeptide can be isolated by affinity chromatography after concentration of the culture supernatant.

Alternatively, when the antibody-producing cells contain a polypeptide or a polypeptide into which a mutation is introduced in the cytoplasm, the polypeptide can be isolated by purification using affinity chromatography or the like after preparation of cell-free extracts.

The present invention also relates to methods for producing a DNA encoding a polypeptide or a polypeptide into which a mutation is introduced, which comprise the steps of:

(a) producing a DNA encoding a polypeptide or a polypeptide into which a mutation is introduced, by the above method for producing a DNA encoding a polypeptide or a polypeptide into which a mutation is introduced; and

(b) collecting the polypeptide encoded by the DNA produced in step (a).

Once a DNA encoding a desired polypeptide is obtained, those skilled in the art can prepare the polypeptide from the DNA by well-known methods. Such methods include, but are not limited to, for example, a method in which a gene expression vector carrying the DNA as an insert is introduced into cells to collect the polypeptide expressed in the cells.

The present invention also provides an antibody-producing cell in which homologous recombination has occurred between a region comprising a DNA encoding an antibody variable region and a DNA construct, wherein the DNA construct comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct.

The DNA constructs contained in the cells of the present invention are described above.

The cells of the present invention may have all of the characteristics described herein. Such cells include, but are not limited to, the cells described below.

Antibody-producing cells comprising the DNA constructs of (a) to (c) or (a) to (d) below, in which homologous recombination has occurred between a region comprising a DNA encoding an antibody variable region and the DNA constructs of (a) below, and whose endogenous AID gene is functionally destroyed:

(a) a DNA construct that comprises

(1) a promoter DNA that functions in the cell;

(2) a DNA that encodes a desired amino acid sequence;

(3) a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;

(b) a DNA construct that comprises

a promoter DNA that functions in the cell; and

a DNA that is located between two site-specific recombinase recognition sequences oriented in opposite directions, and which can be inverted by a site-specific recombinase and comprises an exogenous AID gene;

(c) a DNA construct that comprises a promoter DNA that functions in the cell and a DNA encoding a site-specific recombinase, wherein the site-specific recombinase is activated in the presence of an extracellular stimulus but not in the absence of the stimulus; and

(d) a DNA construct that comprises an XRCC3 gene wherein the sixth exon is inactivated in one of the two alleles of the gene.

The DNA construct of (c) can also be described as a DNA construct that comprises

a promoter DNA that functions in the cell, and a DNA encoding a fusion protein of a site-specific recombinase with an estrogen receptor or a protein comprising an estrogen-binding domain thereof.

Such cells can be prepared by the methods described above.

Cells containing the DNA constructs of (a) to (c) above include, but are not limited to, DT40-SW cells.

The present invention also relates to kits that comprise the cells described herein. The kits can be used, for example, to insert a DNA encoding a desired amino acid sequence into a region comprising a DNA encoding an antibody variable region of antibody-producing cells. Alternatively, the kits can be used to produce antibody-producing cells that produce polypeptides into which mutations are introduced.

The kits of the present invention may comprise a targeting vector described herein in addition to the cells described herein.

The present invention also provides the targeting vectors described herein. The present invention also provides cells comprising a targeting vector of the present invention. The cells of the present invention can be used, for example, to produce cells that produce polypeptides into which mutations are introduced. Such cells include, but are not limited to, those described below.

The present invention also provides kits comprising a targeting vector of the present invention. The kits of the present invention can be used to allow homologous recombination between a region comprising a DNA encoding an antibody variable region of an antibody-producing cell and a DNA construct that comprises:

(1) a promoter DNA that functions in the cell;

(2) a DNA that encodes a desired amino acid sequence; and

(3) a DNA that inhibits the production of a polypeptide comprising the desired amino acid sequence, and can be removed from the DNA construct.

The kits of the present invention may comprise the antibody-producing cells described herein.

The present invention also provides DNAs comprising the nucleotide sequence of SEQ ID NO: 7. Such DNAs are useful as materials for constructing the gene targeting vectors of the present invention, and can be isolated from chicken-derived antibody-producing cells, for example, by the methods described in the Examples.

The sequences newly identified in the present invention are the nucleotides of positions 1 to 3120 and positions 3882 to 7891 in the nucleotide sequence of SEQ ID NO: 7.

The present invention also provides vectors into which a DNA of the present invention is inserted. For example, when E. coli is used as a host, there is no particular limitation on the vectors of the present invention, as long as they have an “ori” for amplification in E. coli (for example, JM109, DH5alpha, HB101, and XL1Blue) to allow large-scale amplification and preparation of the vectors in E. coli or such, and they further have a gene for selecting transformed E. coli (for example, a drug resistance gene that allows discrimination using an agent such as ampicillin, tetracycline, kanamycin, and chloramphenicol). Such vectors include, for example, M13 vectors, pUC vectors, pBR322, pBluescript, and pCR-Script. For cDNA subcloning and excision, it is possible to use, for example, pGEM-T, pDIRECT, and pT7, in addition to the above vectors. When using vectors to produce a polypeptide encoded by a DNA of the present invention, expression vectors are particularly useful. For example, when the objective is to express in E. coli, the expression vectors need to have the above properties for their amplification in E. coli. Additionally, when E. coli such as JM109, DH5alpha, HB101, and XL1-Blue are used as the host, the vectors need to have a promoter for efficient expression in E. coli, for example, the lacZ promoter (Ward et al., Nature 341, 544-546, 1989; FASEB J. 6, 2422-2427, 1992), araB promoter (Better et al., Science 240, 1041-1043, 1988), or T7 promoter. Such vectors include pGEX-5X-1 (Pharmacia), “QIAexpress system” (QIAGEN), pEGFP, and pET, in addition to the above vectors.

Furthermore, the vectors may contain a signal sequence for polypeptide secretion. When producing polypeptides into the periplasm of E. coli, the pelB signal sequence (Lei, S. P. et al., J. Bacteriol. 169, 4379, 1987) may be used as a signal sequence for polypeptide secretion. The vectors can be introduced into host cells, for example, by calcium chloride methods or electroporation methods.

In addition to E. coli vectors, vectors for expressing DNAs of the present invention include, for example, expression vectors derived from mammals (for example, pcDNA3 (Invitrogen), pEGF-BOS (Nucleic Acids Res. 18 (17), 5322, 1990), pEF, and pCDM8), insect cells (for example, “Bac-to-BAC baculovirus expression system” (GIBCO-BRL) and pBacPAK8), plants (for example, pMH1 and pMH2), animal viruses (for example, pHSV, pMV, and pAdexLcw), retroviruses (for example, pZIPneo), yeasts (for example, “Pichia Expression Kit” (Invitrogen), pNV11, and SPQ01), and Bacillus subtilis (for example, pPL608 and pKTH50).

For expression in animal cells such as CHO, COS, and NIH3T3 cells, the vectors need to have a promoter necessary for expression in such cells, for example, the SV40 promoter (Mulligan et al. (1979) Nature 277:108), MMLV-LTR promoter, EF1alpha promoter (Mizushima et al. (1990) Nucleic Acids Res. 18:5322), or CMV promoter. It is more preferred that the vectors have a gene for selecting transformants (for example, drug-resistance genes that allow discrimination using a drug such as neomycin and G418). The vectors having such properties include, for example, pMAM, pDR2, pBKRSV, pBK-CMV, pOPRSV, and pOP13.

DNAs of the present invention can be introduced into cells by methods known to those skilled in the art, for example, electroporation methods.

The present invention also provides cells into which a DNA or vector of the present invention is introduced. There is no particular limitation on the host cells into which a vector of the invention is introduced. It is possible to use, for example, E. coli and various types of animal cells. The host cells of the present invention can be used, for example, as a production system for producing and expressing polypeptides of the present invention. Polypeptide production systems include in vitro and in vivo systems. Such in vitro production systems include those using eukaryotic cells or prokaryotic cells.

Eukaryotic cells, for example, animal cells, plant cells, and fungi cells can be used as the host. Known animal cells include, for example, mammalian cells such as CHO (J. Exp. Med. (1995) 108:945), COS, 3T3, myeloma, BHK (baby hamster kidney), HeLa, and Vero; amphibian cells such as Xenopus oocytes (Valle et al. (1981) Nature 291, 358-340); and insect cells such as Sf9, Sf21, and Tn5. In particular, for CHO cells, dhfr-CHO which is deficient in the DHFR gene (Proc. Natl. Acad. Sci. USA (1980) 77:4216-4220) and CHO K-1 (Proc. Natl. Acad. Sci. USA (1968) 60:1275) can be preferably used. Of animal cells, CHO cells are particularly preferable for large-scale expression. Vectors can be introduced into host cells, for example, by calcium phosphate methods, DEAE-dextran methods, methods using cationic liposome DOTAP (Boehringer-Mannheim), electroporation methods, lipofection methods, etc.

Plant cells include, for example, Nicotiana tabacum-derived cells are known as a polypeptide production system. It is possible to use callus cultures from these cells. Known fungal cells include yeast cells, for example, the genus Saccharomyces such as Saccharomyces cerevisiae, and filamentous fungi such as the genus Aspergillus including Aspergillus niger.

Production systems using prokaryotic cells include those using bacterial cells. Known bacterial cells include E. coli (for example, JM109, DH5alpha, and HB101) and Bacillus subtilis.

All prior art documents cited in this specification are incorporated herein by reference.

### EXAMPLES

Hereinbelow, the present invention will be specifically described with reference to the Examples, but the technical scope of the present invention is not to be construed as being limited thereto.

The present inventors and other research groups demonstrated that genes of interest are mutated when they are integrated into the antibody gene locus of DT40 or Ramos cells (Wang, L., et al. Proc. Natl. Acad. Sci. USA 101, 16745-16749 (2004); Kanayama, N., et al. Nucleic Acids Res. 34, e10 (2006); Arakawa, H., et al. Nucleic Acids Res. 36, e1 (2008)). Based on the fact, the present inventors considered that, if a system for introducing foreign antibody genes into DT40 that has the following characteristics is established, efficient affinity maturation of foreign antibodies can be achieved using the DT40-based antibody producing system.

(1) Mutations occur in introduced antibody genes.

(2) Antibodies encoded by introduced genes are expressed and displayed on the cell surface.

(3) Antibodies encoded by introduced genes are expressed and secreted into the culture supernatant.

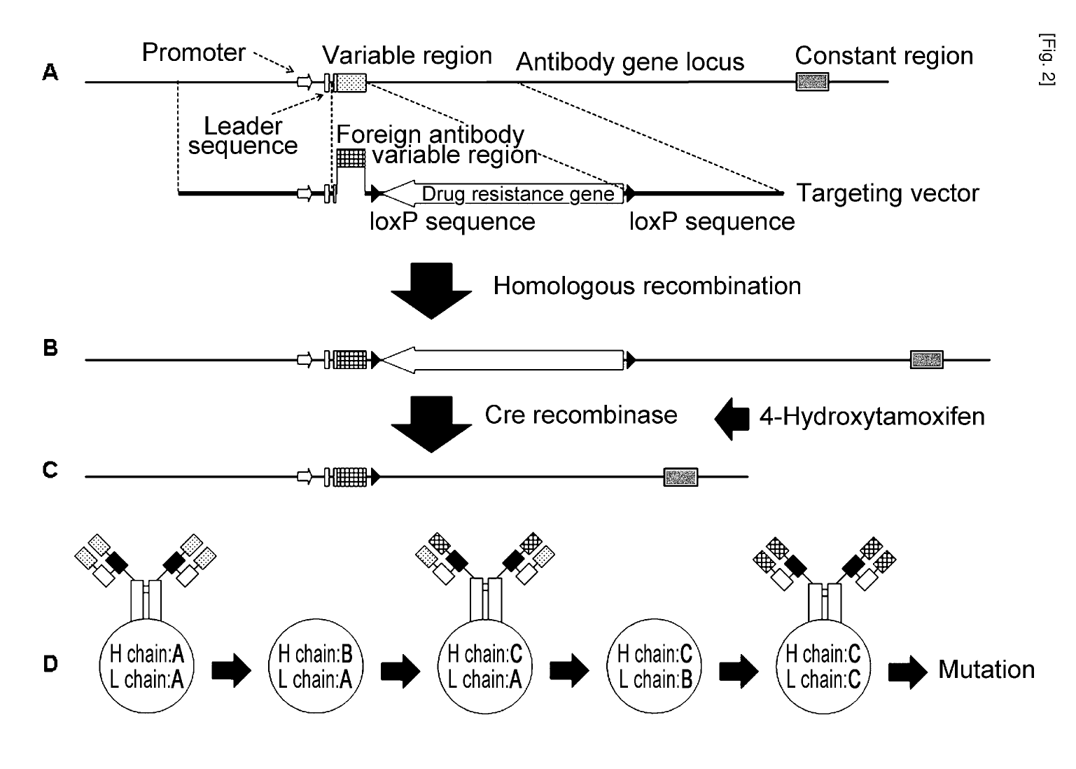

The antibody gene locus is composed of a promoter and downstream exons including: an exon encoding the leader peptide containing a signal required for endoplasmic reticulum targeting; an exon encoding the variable region; and an exon encoding the constant region (FIG. 1). In the heavy chain, the constant region is composed of multiple exons. Whether an antibody is expressed in a secretory form or membrane-bound form is determined depending on the usage of exons. Thus, the present inventors conceived that the most efficient method for constructing a system that has the above characteristics is substituting the variable region of a foreign antibody gene for the variable region exons in the chicken antibody gene locus (FIG. 1A). The present inventors considered that DT40 that expresses a chimeric antibody having a foreign antibody-derived variable region and a chicken-derived constant region can be produced by substituting a foreign antibody-derived variable region for the variable region of each of the heavy-chain and light-chain genes (FIG. 2).

Production of DT40 expressing a chimeric antibody requires the following:

(1) isolation and structural analysis of a chicken antibody gene; and

(2) construction of a targeting vector for substituting the variable region exons in the chicken antibody gene locus. As the genome analysis has advanced, the nucleotide sequences are available for the antibody light chain gene regions; however, only partial information is available on the heavy chain. Thus, the present inventors isolated and sequenced unidentified antibody heavy-chain regions. Based on the revealed information, the present inventors constructed various targeting vectors for substitution of antibody variable region genes, and introduced them into DT40-SW cells. However, the efficiency of obtaining cells with gene modification of interest was found to be lower than that in conventional gene knockout. Then, the present inventors discovered an effective solution and found that cells that efficiently produce chimeric antibodies can be established only by the method of the present invention. Furthermore, the present inventors tried introducing mutations into foreign antibody genes by using the mutation mechanism of the prepared cells, and successfully introduced mutations into the antibody genes with an efficiency comparable to that of wild-type DT40-SW to introduce mutations into its endogenous antibody genes.

The Examples of the present invention are illustrated in detail below.

(1) Cloning and Analysis of the Chicken Antibody Heavy-Chain Gene

Gene fragments upstream and downstream of a variable region gene are required to construct targeting vectors for the variable region gene. The chicken antibody heavy-chain gene has been analyzed to some extent (Reynaud, C-A., et al. Cell 59, 171-183 (1989); Kitao, H., Immunol. Lett. 52, 99-104 (1996); Kitao, H. et al., Int. Immunol. 12, 959-968 (2000)). However, there is little information on the regions around the variable region gene. The antibody heavy chain gene is considered to be very unstable (Reynaud, C-A., et al. Cell 59, 171-183 (1989)). However, the reason remains unclear. In the present invention, the chicken antibody heavy-chain variable region gene was isolated from a genomic DNA library (provided by Dr. Shunichi Takeda of Kyoto University) prepared from DT40.

To prepare probes, the region upstream of the antibody heavy-chain variable region gene was isolated by the DNA walking method. PCR was carried out in 25 micro 1 of reaction mixture using KOD-plus DNA polymerase (TOYOBO). In the first-round PCR, a primer

(cJH1R1: 5′-GGGGTACCCGGAGGAGACGATGACTTCGG-3′; SEQ ID NO: 1)

for a region downstream of JH was used as an antisense primer, while various sequences were used as a sense primer. Using the DT40 genomic DNA as a template, PCR was carried out as follows: 15 cycles of: [94 degrees C. for 15 sec, 68 degrees C. (−0.7 degrees C./cycle) for 30 sec, and 68 degrees C. 4 min] followed by 15 cycles of: [94 degrees C. for 15 sec, 57 degrees C. for 30 sec, and 68 degrees C. for 4 min]. The second-round PCR used 5 micro 1 of the first-round PCR mixture as a template, an antisense primer

(cJH-R: 5′-CTTCGGTCCCGTGGCCCCATGCGTCGAT-3′; SEQ ID NO: 2),

and the same sense primer as the first-round PCR. The second-round PCR was carried out with 30 cycles of: [94 degrees C. for 15 sec, 62 degrees C. for 30 sec, and 68 degrees C. for 4 min]. PCR using

TdT-2 (5′-GGTTCAATGTAGTCCAGTCC-3′; SEQ ID NO: 3)

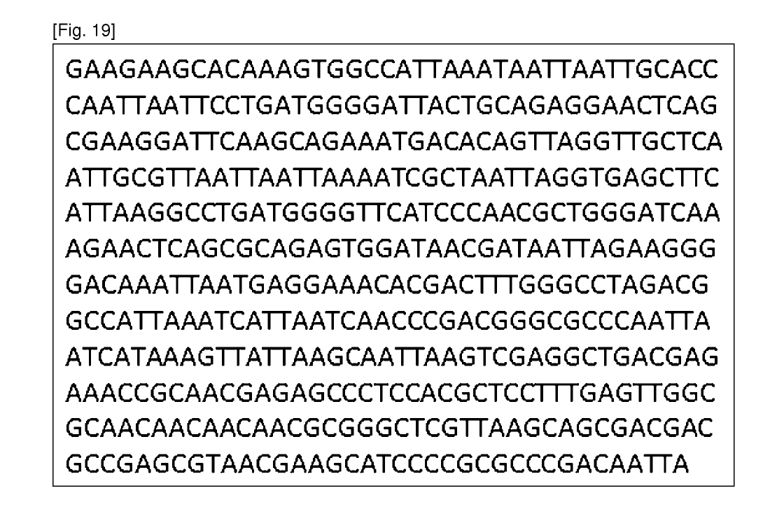

as a sense primer yielded a PCR product of about 1 kbp. Thus, the product was cloned into the pCR-Blunt vector (Invitrogen). The sequence analysis of the product revealed that it contained a chicken antibody heavy chain VDJ gene sequence as well as a known sequence in the region upstream of the variable region gene (M30319; Reynaud, C-A., et al. Cell 59, 171-183 (1989)) and a further upstream sequence of 464 by (FIG. 19; SEQ ID NO: 4). To clone genes in the regions around the antibody heavy chain variable region, a DIG-labeled probe was prepared using a PCR DIG Probe Synthesis Kit (Roche), together with the above gene fragment as a template, and a sense primer

(CHCupF-Xba: 5′-GTGGCCATTCTAGAATTAATTGCACC-3; SEQ ID NO: 5),

and an antisense primer

(CHCupR-Bam: 5′-GGAGGGATCCGGCTTCGTTAGC-3′; SEQ ID NO: 6).

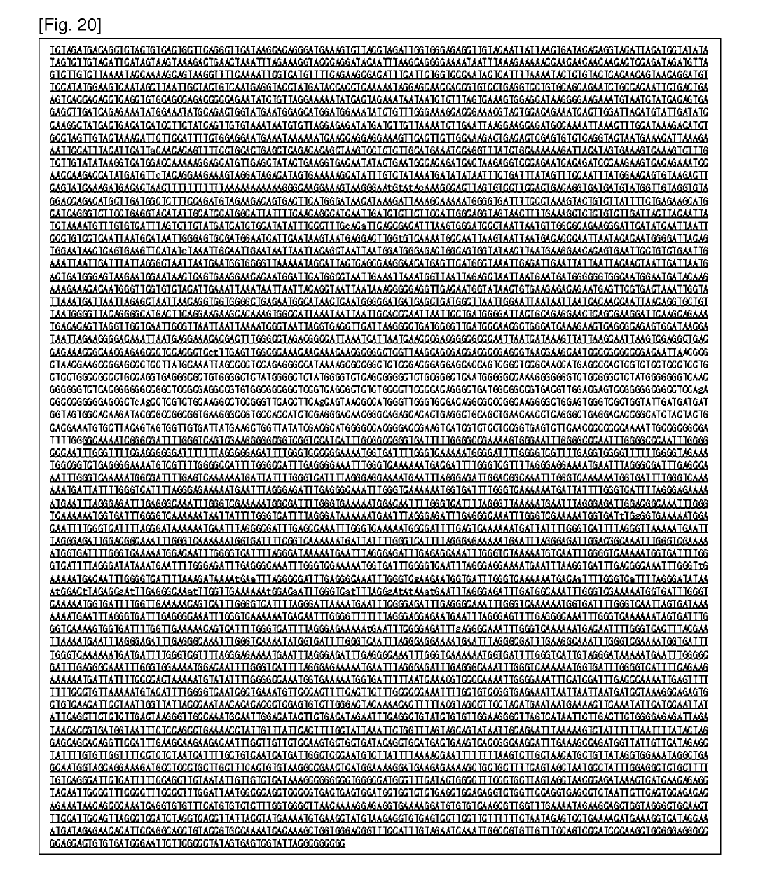

The lambda DASH II phage library, into which fragments of about 20 kbp from the DT40 genomic DNA are inserted, was screened using the above probe according to the standard protocol provided by Roche. Two positive clones were obtained from a phage library (200,000 pfu). A NotI fragment of about 20 kbp derived from one of the two clones was subcloned into pBluescript II SK. Then, the XbaI-NotI fragment (a total of about 8 kbp) containing the heavy-chain variable region gene and its upstream (about 3.5 kbp) and downstream (about 4.0 kbp) regions was obtained from the subclone. A restriction map (FIG. 3) was constructed for the XbaI-NotI fragment, and the sequence was analyzed. The result showed that the fragment contains the chicken antibody heavy-chain gene of 7891 bp (FIG. 20; SEQ ID NO: 7). The entire region, excluding the region of 761 bps around the variable region gene including the sequence reported by Reynaud, C-A et al. (Cell 59, 171-183 (1989)), was found to be a novel sequence previously unreported. This sequence was used to construct targeting vectors for the heavy-chain variable region gene.

(2) Construction of Gene Targeting Vectors for the Heavy Chain Variable Region

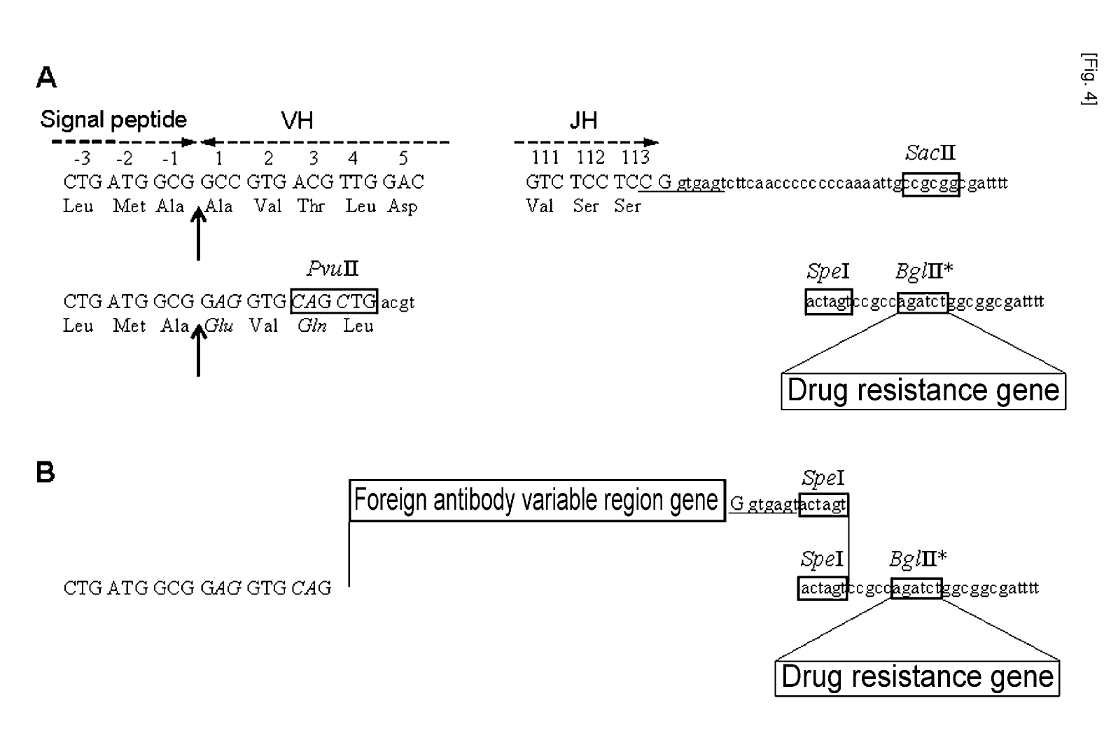

To insert a foreign antibody variable region gene, restriction sites were introduced around the signal peptide sequence and in the intron downstream of JH (FIGS. 4 to 6, 21, and 22). Two types of targeting vectors were constructed, which differ in the restriction sites introduced in the signal peptide sequence.

(VH targeting vector 1; FIGS. 4, 5, and 21)

The codons encoding the third and fourth amino acids of VH were modified to constitute a PvuII site (FIG. 4A). This results in alteration of the pair of encoded amino acids from threonine/leucine to glutamine/leucine. Furthermore, the first amino acid of VH was substituted with glutamic acid, which is relatively frequently used in mouse monoclonal antibodies. In addition, an SpeI site was introduced downstream of JH. Since PvuII digestion yields a blunt end, the 5′ end of the foreign antibody variable region gene to be inserted is designed to be a blunt end. Furthermore, a splicing donor consensus sequence and an SpeI site

(5′-ggtgagtactagt-3′; SEQ ID NO: 42)

were attached to the 3′ end of JH (FIG. 4B). Alternatively, instead of an SpeI site, an AvrII, XbaI, or NheI site may be attached to the foreign antibody variable region gene, because digestion with AvrII, XbaI, or NheI produces the same cohesive end as SpeI digestion. The above design allows insertion of a wide range of antibody gene fragments.

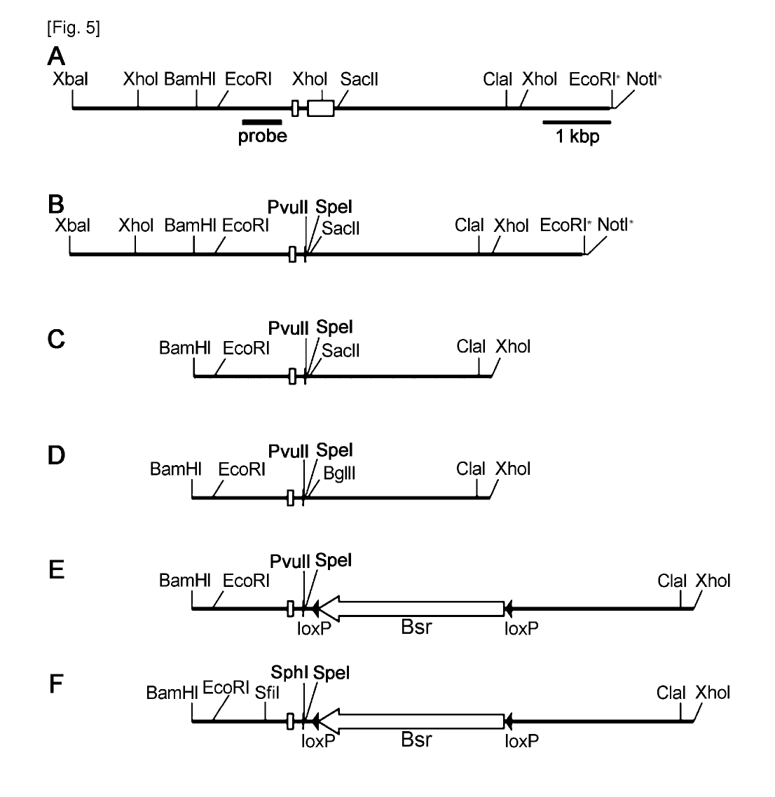

The XbaI-NotI fragment of the chicken antibody heavy chain gene was inserted via an oligonucleotide linker into the pBluescript II SK vector that was digested in advance with PvuII to remove the multicloning site (FIG. 5A). A PCR fragment was prepared using the chicken antibody heavy-chain gene as a template, together with a sense primer

(CHFup1k-Bam: 5′-GTGGGATCCCTAATTAATGTTGGCG-3′; SEQ ID NO: 8) near the BamHI site and an antisense primer

(CJH4R-PSS: 5′-TATTCCGCGGACTAGTACGTCAGCTG AACCTCCGCCATCAGCCCTGTGGGGA-3′; SEQ ID NO: 9)

near the signal peptide sequence, which contains PvuII, SpeI, and SacII sites. The PCR fragment was substituted for the BamHI-SacII portion of the mouse antibody heavy chain gene (FIG. 5B). The upstream XbaI-BamHI and downstream XhoI-NotI portions were removed in succession (FIG. 5C). A BglII site was introduced at the SacII site using a linker

(5′-CAGATCTGGC-3′; SEQ ID NO: 43) (FIG. 5D).

A DNA fragment containing the beta-actin promoter DNA, blasticidin S-resistance gene, and poly A addition sequence that are located between loxP sequences, was excised from pLoxBsr (Arakawa, H., et al. BMC Biotechnol. 17 (2001)) using BamHI, and inserted into the BglII site (FIG. 5E). Other DNA constructs may be used as long as they contain a promoter DNA and a marker gene such as a drug-resistance gene located between loxP sequences. There is no limitation on the orientation of the DNA construct containing a promoter DNA and a marker gene located between loxP sequences, relative to the target gene. For the vector used in this Example, the blasticidin S-resistance gene was inserted in the opposite orientation relative to the direction of transcription of the antibody heavy-chain gene. The sequence of VH targeting vector 1 is shown in FIG. 21 (SEQ ID NO: 10).

(VH targeting vector 2; FIGS. 5, 6, and 22)

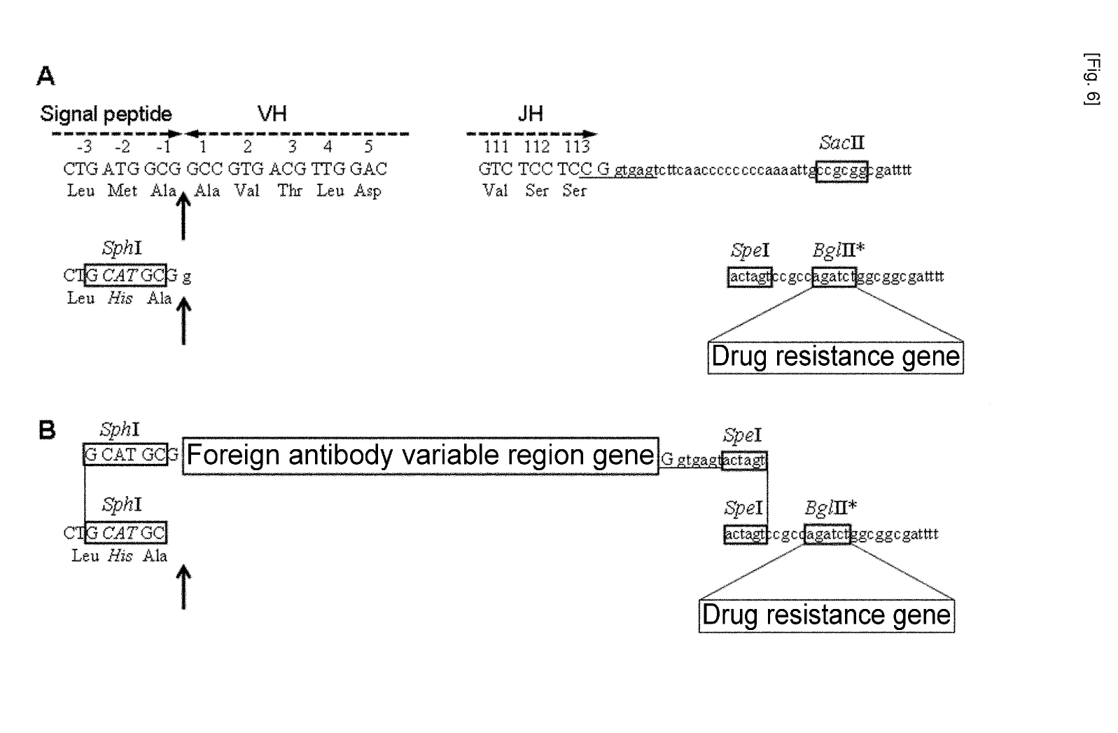

To allow insertion of a foreign antibody variable-region gene immediately after the signal peptide, the nucleotide sequence of the end of the signal peptide was modified to have an SphI site (FIG. 6A). The structure on the JH side was the same as VH targeting vector 1. When a foreign antibody gene is inserted into the vector without altering the sequence of the structural gene region, an SphI site and a nucleotide “g” (5′-gcatgcg-3′) are attached to the 5′ end of the gene, and a splicing donor consensus sequence and an SpeI site

(5′-ggtgagtactagt-3′; SEQ ID NO: 42)

are attached to the 3′ end, as in the case of VH targeting vector 1 (FIG. 6B). Alternatively, instead of an SpeI site, an AvrII, XbaI, or NheI site may be attached to the foreign antibody variable-region gene, because digestion with AvrII, XbaI, or NheI produces the same cohesive end as SpeI digestion.

A gene fragment was prepared by PCR using the chicken antibody heavy-chain gene as a template, together with a sense primer for the upstream region (VHupF2: 5′-TTAGAAGGGGACAAATTAATGAGGAAACACGACTTTGG-3′; SEQ ID NO: 11)

and an antisense primer having SpeI and SphI sites

(VHupR2: 5′-CGACTAGTCCGCATGC AGCCCTGTGGGGAAGGGCAGAGAGCGCTGAC-3′; SEQ ID NO: 12).

The fragment was digested at the SfiI and SpeI sites therein, and was substituted for the SfiI-SpeI fragment of VH targeting vector 1 (FIG. 5F). There is no limitation on the orientation of the DNA construct containing a promoter DNA and a marker gene located between loxP sequences, relative to the target gene. For the vector used in this Example, the blasticidin S-resistance gene was inserted in the opposite orientation relative to the direction of transcription of the antibody heavy chain gene. The sequence of VH targeting vector 2 is shown in FIG. 22 (SEQ ID NO: 13).

(3) Construction of Gene Targeting Vectors for the Light-Chain Variable Region

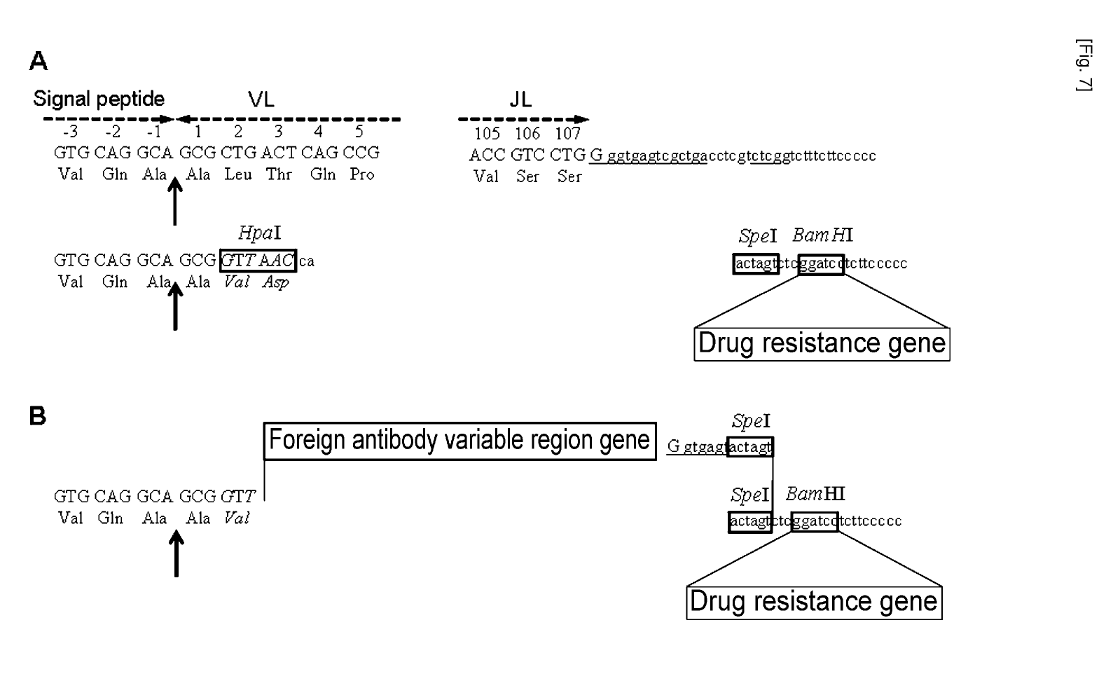

As in the case of the antibody heavy chain, to insert a foreign antibody variable region gene, restriction sites were introduced around the signal peptide sequence and in the intron downstream of JL (FIGS. 7-9, 23, and 24). Two types of targeting vectors were constructed, which differ in the restriction sites introduced in the signal peptide sequence.

(VL targeting vector 1; FIGS. 7, 8, and 23)

The codons encoding the second and third amino acids of VL were modified to constitute an HpaI site (FIG. 7A). This results in alteration of the pair of encoded amino acids from leucine/threonine to valine/aspartic acid. In addition, an SpeI site was introduced downstream of JL as described above for the VH targeting vectors. Since HpaI digestion yields a blunt end, the 5′ end of the foreign antibody variable region gene to be inserted is designed to be a blunt end. Furthermore, a splicing donor consensus sequence and an SpeI site

(5′-ggtgagtactagt-3′; SEQ ID NO: 42)

were attached to the 3′ end of JL (FIG. 7B). Alternatively, instead of an SpeI site, an AvrII, XbaI, or NheI site may be attached to the foreign antibody variable region gene, because digestion with AvrII, XbaI, or NheI produces the same cohesive end as SpeI digestion. The above design allows insertion of a wide range of antibody gene fragments.

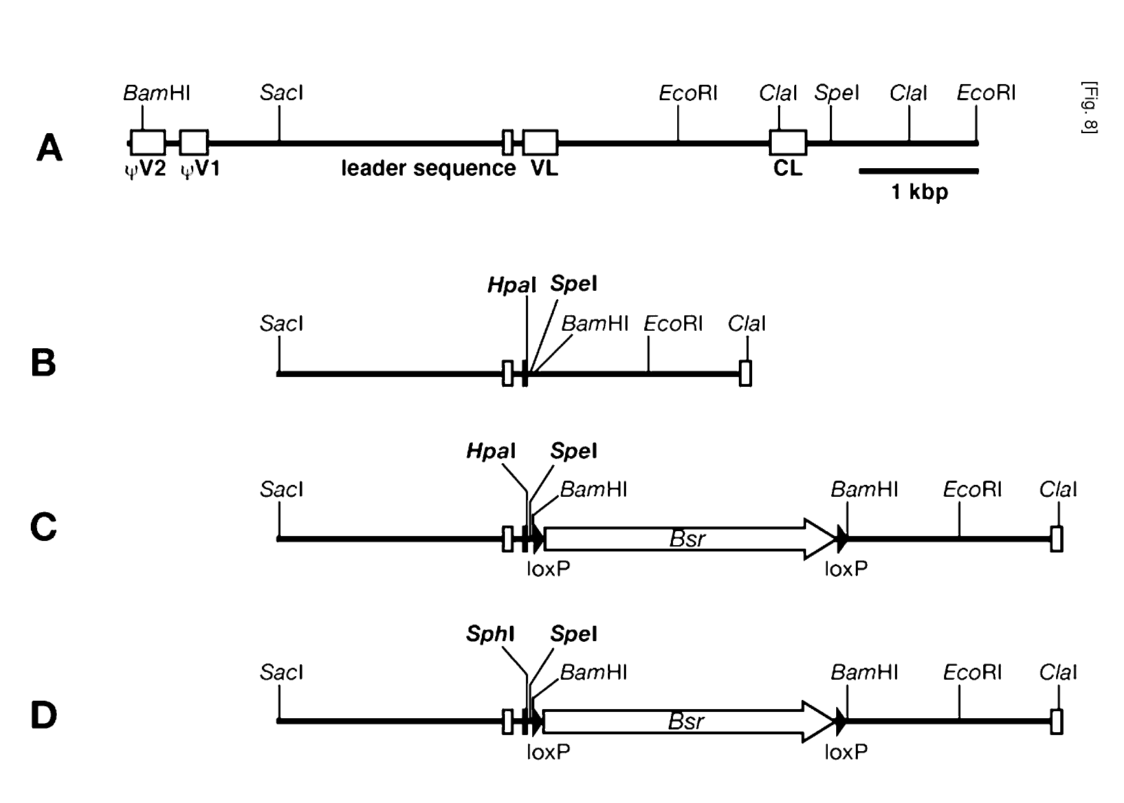

The chicken light-chain gene has already been cloned (Reynaud, C.-A., et al., Cell 40, 283-291 (1985)), and the nucleotide sequences around the gene have also been revealed by genomic analysis (International Chicken Genome Sequencing Consortium, Nature 432, 695-716 (2004)) (FIG. 8A).

Thus, utilizing the primers described below, gene fragments were prepared by PCR using the genomic DNA extracted from DT40 cells as a template. The 5′ upstream region of the antibody light-chain variable region gene was amplified using a sense primer

(IgLU53: 5′-ACGACCCTGGCACCAACAGAGACCTGC-3′; SEQ ID NO: 14), and an antisense primer

(IgLU32: 5′-ACTAGTTGGTTAACCGCTGCCTGCACCAGGGAACCTGGAG-3′; SEQ ID NO: 15)

having HpaI and SpeI sites. The 3′ downstream region was amplified using a sense primer

(IgLD53: 5′-ACTAGTCTCGGATCCTCTTCCCCCATCGTGAAATTGTGAC-3′; SEQ ID NO: 16)

having SpeI and BamHI sites, and an antisense primer (IgLD34: 5′-AGCGGGTGGAGCCATCGATGACCCAATCCACAGTCA-3′; SEQ ID NO: 17).

The resulting PCR products were cloned into the pCR-Blunt vector (Invitrogen). The 5′-side fragment was excised with SacI and SpeI, and inserted into a vector containing the 3′-side fragment as an insert (FIG. 8B). A DNA fragment (Arakawa, H., et al. BMC Biotechnol. 17 (2001)) containing a beta-actin promoter DNA, the blasticidin S-resistance gene, and a poly A addition sequence located between loxP sequences, or a DNA fragment (Arakawa, H., et al. PLoS Biol., 2E179 (2004)) containing a beta-actin promoter DNA, the xanthine-guanine phosphoribosyltransferase gene, and a poly A addition sequence located between loxP sequences, was excised with BamHI and inserted at the BamHI site downstream of the SpeI site (FIG. 8C). Other DNA constructs may be used as long as they contain a promoter DNA and a marker gene such as a drug-resistance gene located between loxP sequences. There is no limitation on the orientation of the DNA construct containing a promoter DNA and a marker gene located between loxP sequences, relative to the target gene. For the vector used in this Example, a marker gene was inserted in the same orientation as the direction of transcription of the antibody light chain gene. The sequence of VL targeting vector 1 is shown in FIG. 23 (SEQ ID NO: 18).

(VL targeting vector 2: FIGS. 8, 9, and 24)

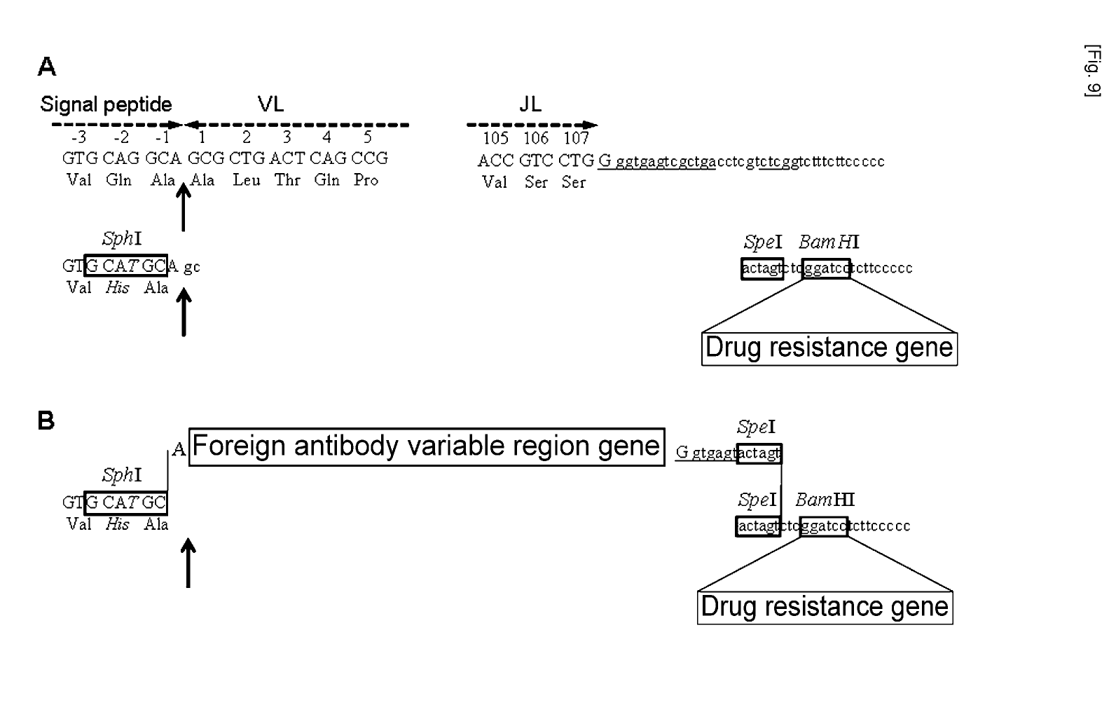

As in the case of VH targeting vector 2, to allow insertion of a foreign antibody variable region gene immediately after the signal peptide, the nucleotide sequence of the end of the signal peptide was modified to have an SphI site (FIG. 9A). The structure on the JL side was the same as VL targeting vector 1. When a foreign antibody gene is inserted into the vector without altering the sequence of the structural gene region, an SphI site and a nucleotide “a” (5′-gcatgca-3′) are attached to the 5′ end, and a splicing donor consensus sequence and an SpeI site

(5′-ggtgagtactagt-3′; SEQ ID NO: 42)

are attached to the 3′ end as in the case of VL targeting vector 1 (FIG. 9B). Alternatively, instead of an SpeI site, an AvrII, XbaI, or NheI site may be attached to the foreign antibody variable region gene, because digestion with AvrII, XbaI, or NheI produces the same cohesive end as SpeI digestion.

As in the case of VL targeting vector 1, a gene fragment was prepared by PCR using the genomic DNA of DT40 as a template, together with the above-described upstream sense primer for the 5′ region, and an antisense primer

(IgLU33: 5′-GAACTAGTGCTGCATGCACCAGGGAACCTGGAGAGGGAG-3′; SEQ ID NO: 19)

having SpeI and SphI sites, and then this was cloned into the pCR-Blunt vector. The cloned fragment was excised with SacI and SpeI, and substituted for the SacI-SpeI fragment of VL targeting vector 1 (FIG. 8D). There is no limitation on the orientation of the DNA construct containing a promoter DNA and a marker gene located between loxP sequences, relative to the target gene. For the vector used in this Example, a marker gene was inserted in the same orientation as the direction of transcription of the antibody light chain gene. The sequence of VL targeting vector 2 is shown in FIG. 24 (SEQ ID NO: 20).

(4) Introduction and Mutagenesis of a Mouse Monoclonal Antibody Variable Region Gene

As a model, the variable region gene derived from the monoclonal antibody 17.2.25 against 4-hydroxy-3-nitrophenylacetyl (NP), a hapten, was introduced into DT40 via a vector, and the cells were assessed for antibody expression and mutations.

(Introduction of a Mouse Antibody Heavy-Chain Variable Region Gene)

Using TRIzol (Invitrogen), total RNA was extracted from an anti-NP IgM antibody-producing hybridoma (Kanayama, N., et al., J. Immunol. 169, 6865-6874 (2002)) prepared from spleen cells of mice in which the antibody heavy chain of the anti-NP monoclonal antibody 17.2.25 is knocked in (Quasi-monoclonal mice, Cascalho, M., et al., Science 272, 1649-(1996)). cDNA was synthesized from the RNA using Superscript II reverse transcriptase (Invitrogen) and an oligo-dT primer. The anti-NP antibody heavy-chain variable region (VHT) was amplified by PCR using the cDNA as a template, together with a sense primer

(VHTF: 5′-GAGGTTCAGCTGCAGCAGTCTGGG-3′; SEQ ID NO: 21), and an antisense primer

(VHT-Spe_S: 5′-ACTAGTACTCACCTGAGGAGACGGTGACT-3′; SEQ ID NO: 22)

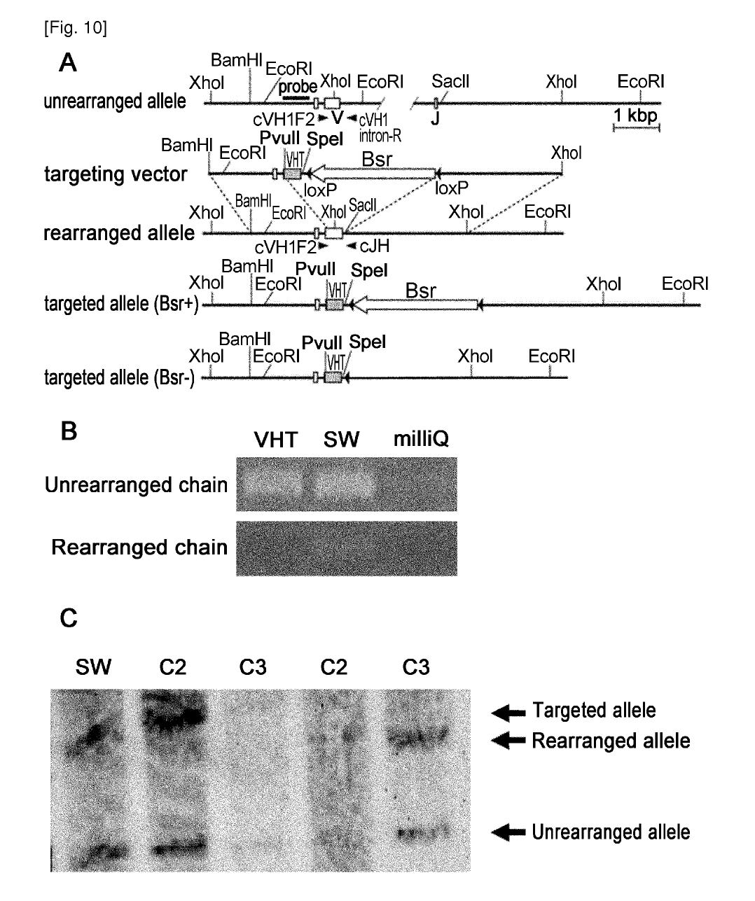

having an SpeI site. The PCR fragment was digested with SpeI, and this was inserted into VH targeting vector 1 digested with PvuII and SpeI (FIG. 10A).

DT40-SW, which was produced by modification of the DT40 chicken B cell line having the ability to spontaneously introduce mutations into antibody genes, was used as the host cell for introduction of the constructed targeting vector. DT40-SW has the following characteristics.

(i) When a DNA construct for expression of a Cre recombinase/estrogen receptor fusion protein gene under the control of the CMV promoter is introduced, the cells constitutively express an inactive Cre recombinase/estrogen receptor fusion protein.

(ii) One of the two alleles of the endogenous AID gene locus is deficient, and the other has been substituted with an AID gene that is expressed by the CAG promoter and located between two loxP sequences oriented in opposite directions.

(iii) The AID gene is linked to, in the order of, an IRES, the GFP gene and a poly A addition sequence in the same orientation as the AID gene, and a poly A addition sequence and the puromycin-resistance gene in the opposite orientation relative to the AID gene, and this is inserted between two loxP sequences.

(iv) Upon addition of 4-hydroxytamoxifen to the cells, the Cre recombinase of (i) is activated, and inverts the DNA construct of (ii) or (iii) which contains the AID gene and is located between the loxP sequences. Thus, the AID gene is expressed when it is oriented in the same direction as the CAG promoter, but the gene is not expressed when it is oriented in the opposite direction relative to the promoter.

(v) Cells that express the AID gene can be isolated as GFP gene-expressing cells, while cells that do not express the AID gene can be selected based on the drug resistance by expression of the puromycin-resistance gene (Kanayama, N., et al. Biochem. Biophys. Res. Commun. 327, 70-75; JP-A (Kokai) 2006-109711).

Targeting vectors were transfected into DT40-SW by the following method. 15 micro g of a vector was linearized by BamHI digestion, and mixed with 1×10⁷ cells of DT40-SW. 500 micro 1 of the resulting suspension was placed into an electroporation cuvette with a 4-mm gap. Electroporation was carried out at 550 V and 25 micro F using Gene Pulser Xcell (Bio-Rad). After electroporation, the cells were suspended in 10 ml of growth medium (PRMI 1640 (Invitrogen), 10% fetal bovine serum (Invitrogen), and 1% chicken serum (Sigma)), and cultured for 24 hours. Then, 10 ml of 2× selection medium (growth medium supplemented with blasticidin S (Kaken Pharmaceutical Co.)) was added to the cells. The final concentration of blasticidin S was 20 micro g/ml. The cells were aliquoted into 96-well plates and cultured for 10 to 14 days.

16 clone colonies were obtained by selection using blasticidin S at a final concentration of 20 micro g/ml. Cells that formed the colonies were stained with a phycoerythrin-labeled anti-chicken IgM mouse monoclonal antibody (Southern Biotechnology) and analyzed using FACS Calibur (BD Bioscience). When the gene is successfully targeted, cells become incapable of expressing the antibody heavy chain. Thus, five clones that were negative for staining with the anti-chicken IgM antibody were selected. To assess these clones at the gene level, of the alleles of the endogenous antibody heavy chain gene, the allele resulting from VDJ recombination was amplified using a sense primer

(cVH1F2: 5′-GGCGGCTCCGTCAGCGCTCTCT-3′; SEQ ID NO: 23) and an antisense primer

(cJH-R: 5′-CTTCGGTCCCGTGGCCCCATGCGTCGAT-3′; SEQ ID NO: 2), while the embryonic allele was amplified using the sense primer cVH1F2 and an antisense primer

(cVH1 intron-R: 5′-TTCACCGCCTTGGGTTGCAACGGTGG-3′; SEQ ID NO: 24) (FIG. 10B).

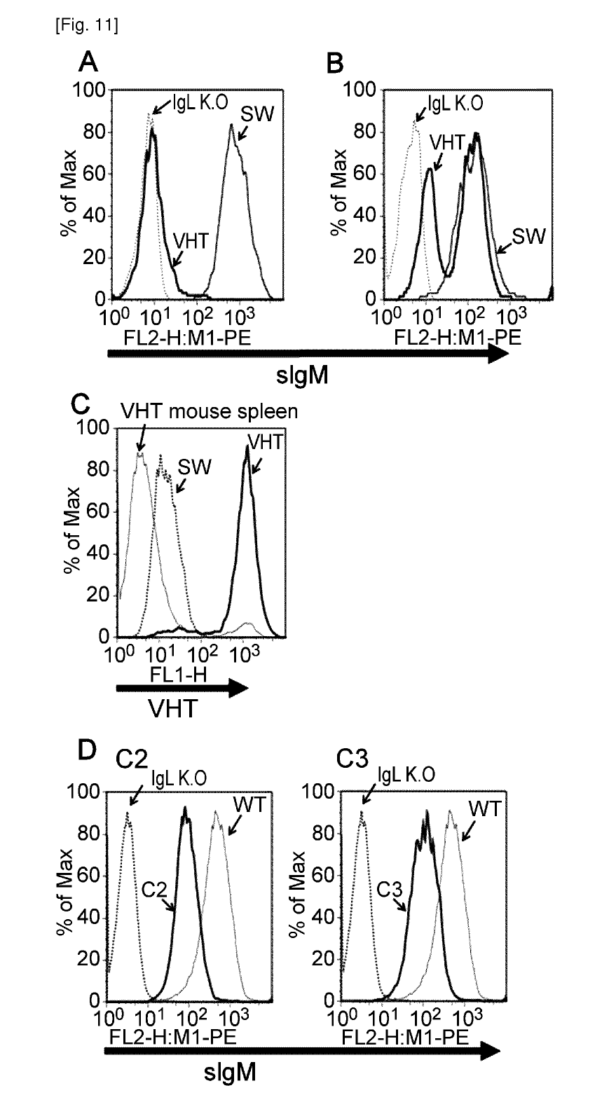

Based on the result, three clones that did not produce the band corresponding to the endogenous heavy-chain variable region gene after VDJ recombination were selected (FIG. 10B). This suggests that the allele resulting from VDJ recombination was targeted with the gene of interest. The targeting was confirmed by genomic Southern blot analysis (FIG. 10C). Genomic DNA was isolated from each clone. After EcoRI digestion, the DNA was electrophoresed and transferred onto a Hybond N nylon membrane (GE Healthcare). Then, detection was carried out using as a probe the DIG-labeled gene fragment which was used to clone the genomic DNA. From the three clones analyzed, clones C2 and C3 were selected because they clearly showed a longer band resulting from insertion of the marker gene. By flow cytometry, the selected clones were confirmed not to produce the antibody (FIG. 11A), as expected from the vector design. Thus, based on the lack of antibody expression on the cell surface, cells into which a foreign gene of interest is introduced can be efficiently prepared by selecting cells in which targeting with the foreign gene has been achieved.

(Expression of the Mouse Antibody Heavy-Chain Variable Region)

When these clones were treated with 50 nM 4-hydroxytamoxifen as previously reported (Kanayama, N., et al. Biochem. Biophys. Res. Commun. 327, 70-75 (2005)), cells with restored expression of the IgM antibody on the cell surface were found by flow cytometry (FIG. 11B), and they lacked the drug resistance. Consistent with this result, the band shift due to loss of the drug-resistance gene was seen in the Southern blot analysis (FIG. 10D). The cells were stained with a VHT CDR3-specific anti-idiotype rat monoclonal antibody (R2.438; provided by Dr. T. Imanishi-Kari) (Kanayama, N., et al. J. Immunol. 169, 6865-6874 (2002)) (FIG. 11C).

The staining intensity observed was comparable to that of mouse B cells into which VHT is integrated, which were used as a positive control. Thus, the cells were demonstrated to express the VHT gene integrated into DT40-SW. After switching on the antibody expression, clones C2 and C3 were subcloned by limiting dilution. The subcloned cells were also analyzed by flow cytometry for the cell-surface antibody expression (FIG. 11D). RNA was isolated from each clone, and cDNA was prepared from the RNA. Gene fragments were amplified by PCR using primers. The splicing between the variable region gene and constant region gene was assessed by sequencing analysis. The result demonstrated that the splicing site attached to the 3′ end of the foreign variable region gene was functioning as designed, and thus the exons of the variable region gene and constant region gene were linked together as expected. That is, it is demonstrated that a foreign antibody heavy-chain variable region gene introduced by the above method can be normally transcribed and translated, and thus expressed as a chimeric antibody with the chicken antibody heavy-chain constant region. Although the surface antibody expression was confirmed, the expression level was slightly lower than that of the wild type. This suggests that the efficiency of expression of the chimeric antibody was reduced because the light chain was derived from a chicken antibody. A possible solution is substitution of the light chain with a light chain corresponding to the foreign antibody heavy chain.

(Introduction of Mutations into the Mouse Antibody Heavy-Chain Variable Region)

To analyze mutations in the VHT gene of the prepared VHT-expressing cells, the mutation mechanism of the cells were activated by switching on the AID gene expression using a method previously reported (Kanayama, N., et al. Biochem. Biophys. Res. Commun. 327, 70-75 (2005)). After treating the cells with 4-hydroxytamoxifen, GFP⁺ cells (i.e., cells whose AID expression which is essential for mutagenesis was switched on) were isolated as single cells by flow cytometry, and cultured for 30 days.

Genomic DNA was isolated from the cultured cells. The heavy-chain variable region was amplified by PCR using the primers

CVH1F2 (5′-GGCGGCTCCGTCAGCGCTCTCT-3′; SEQ ID NO: 25) and

CJH1R2 (5′-GCCGCAAATGATGGACCGAC-3′; SEQ ID NO: 26),

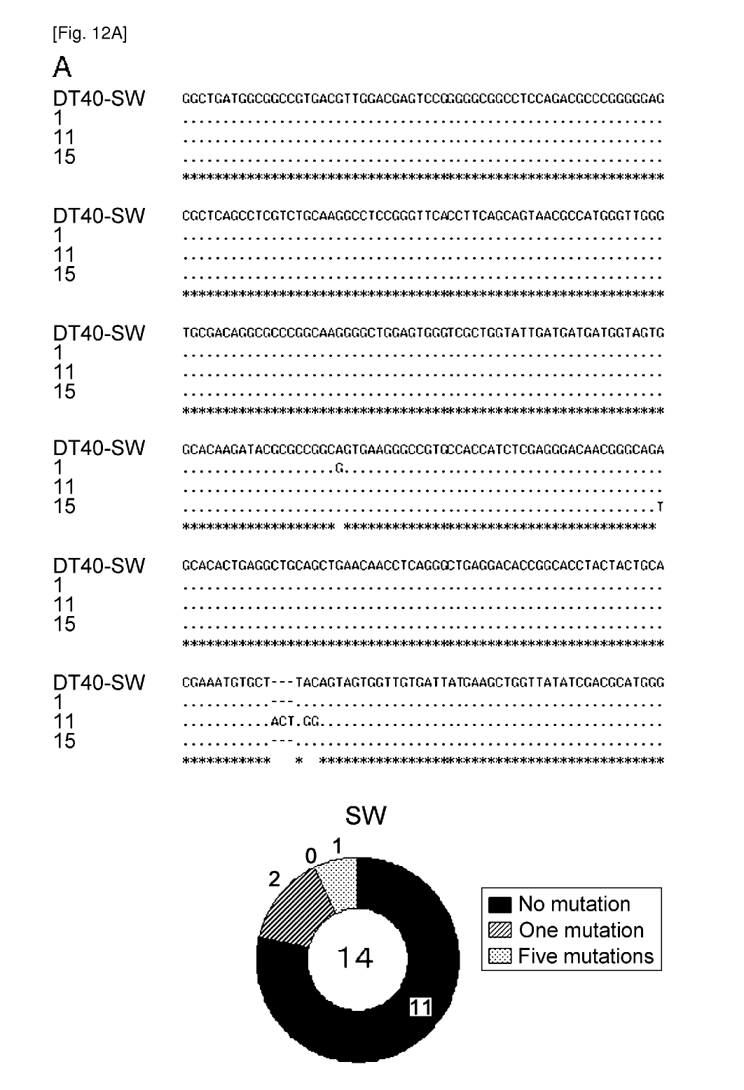

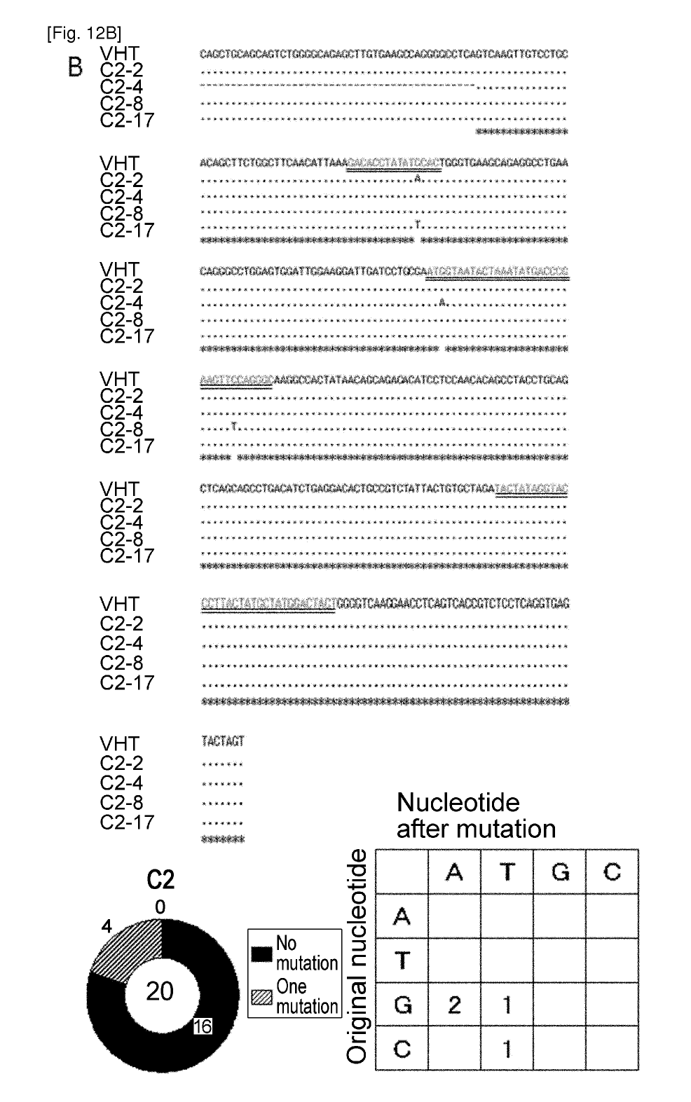

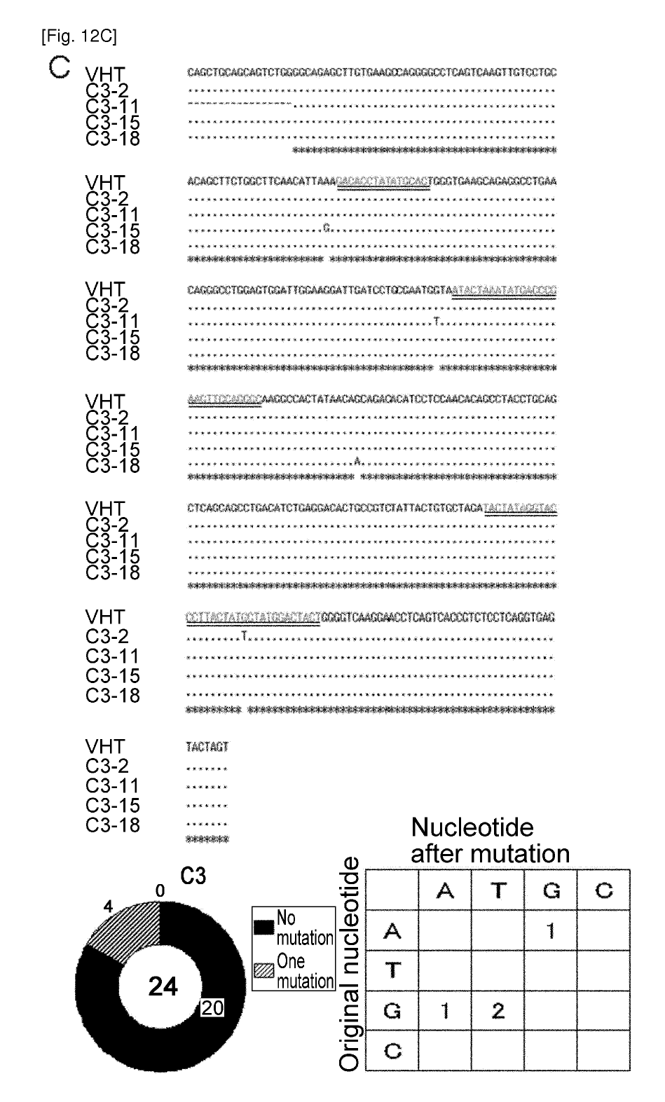

and this was cloned into the pCR-Blunt vector. The clones were analyzed by sequencing. Mutations were observed in clones C2 and C3 (FIGS. 12B and C) at a frequency comparable to that in wild-type DT40-SW (FIG. 12A). That is, it was proven that the above method can be used to introduce mutations and thus modify the variable region gene of an arbitrary foreign antibody introduced into the chicken heavy chain variable region gene locus of DT40. The same effect can be produced by using VH targeting vector 2.

(Introduction of a Mouse Antibody Light-Chain Variable Region Gene)

cDNA was prepared from total RNA of the above-described hybridoma producing an anti-NP IgM antibody. A lambda1 variable region gene fragment was amplified using the cDNA as a template, together with the sense primer

mIgVL51 (5′-ACTCAGGAATCTGCACTCACCACATCACCT-3′; SEQ ID NO: 27) and the antisense primer

mIgVL133 (5′-GTTCTAGACACTCACCTAGGACAGTCAGTTTGGTTCCT-3′; SEQ ID NO: 28)

for the lambda1 light-chain variable region gene. Then, the fragment was digested with XbaI, and inserted into the HpaI-SpeI site of VL targeting vector 1. 15 micro g of a vector was linearized by digestion with SacI, and introduced by electroporation into clone C2 expressing mouse VHT, which was prepared as described above. As in the case of the heavy chain, the antibody expression on the surface of cells from colonies (45 clones) formed under selection with blasticidin S was assessed by flow cytometry. Ten positive clones were obtained by selecting cells lacking the antibody expression. Five clones were selected from the ten clones, and genomic DNA was extracted from the cells. The integration of the targeting vector was assessed by PCR using the following upstream primers:

IgLU-up (5′-TGCCTGGGGTAAGGGTAGTACTCTGTGC-3′; SEQ ID NO: 29) and

cJL12 (5′-AACGGTAGGGGATCCGAGACTAG-3′; SEQ ID NO: 30), and the following downstream primers:

BSR1 (5′-GAGAAAGGTAGAAGACCCCAAGGACTTTCCTTCAGAATTGC-3′; SEQ ID NO: 31) and

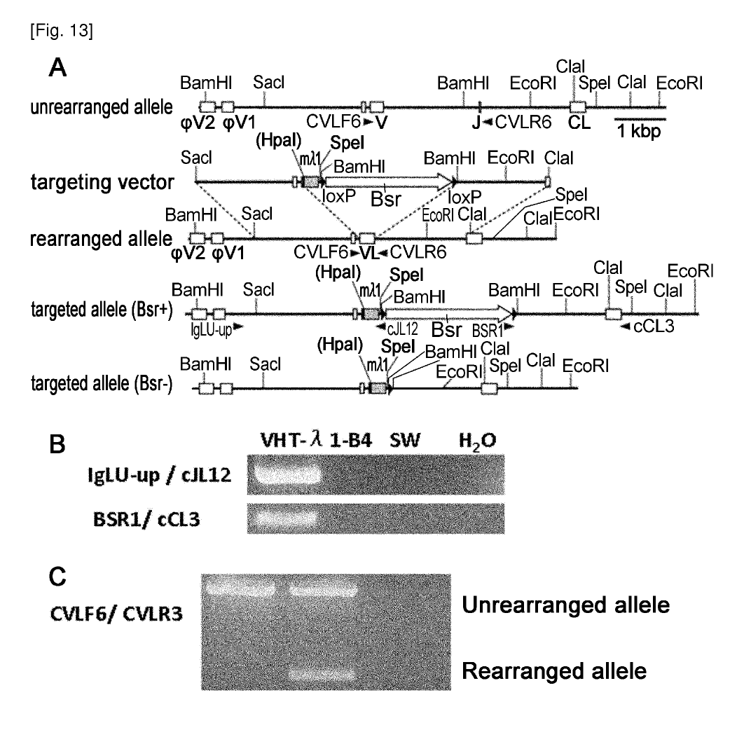

cCL3 (5′-GCAGAGTCAGCACTAGTTCAGTGTCGTGTT-3′; SEQ ID NO: 32) (FIG. 13B).

Furthermore, PCR was used to assess the targeting of the allele that resulted from VJ recombination and producing an antibody using the primers

CVLF6 (5′-CAGGAGCTCGCGGGGCCGTCACTGATTGCCG-3′; SEQ ID NO: 33) and

CVLR3 (5′-GCGCAAGCTTCCCCAGCCTGCCGCCAAGTCCAAG-3′; SEQ ID NO: 34) (FIG. 13C).

Specific amplification of a PCR product was observed in successfully targeted cells (FIG. 13B). Furthermore, clones were selected using band loss due to integration into the antibody production allele as an indicator (FIG. 13C). The result showed that targeting at the desired position with the gene of interest was achieved in all of the clones. In addition, it is demonstrated that cell surface antibody expression is very useful as an indicator for screening for cells of interest.

(Expression of the Antibody Light-Chain Variable Region Gene)

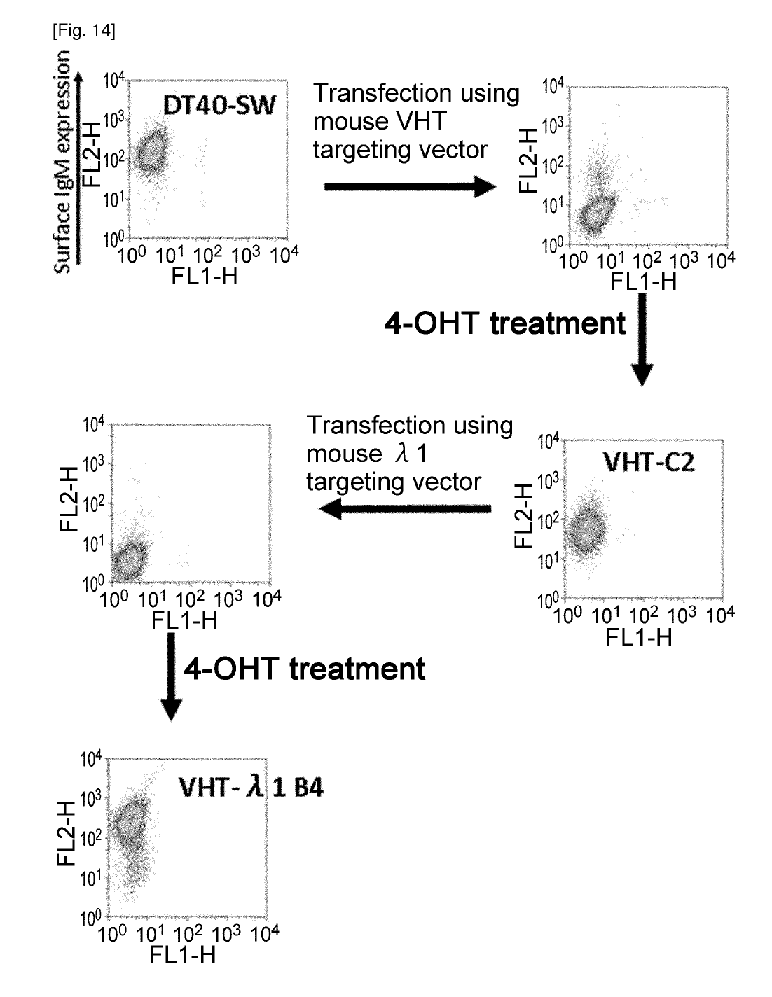

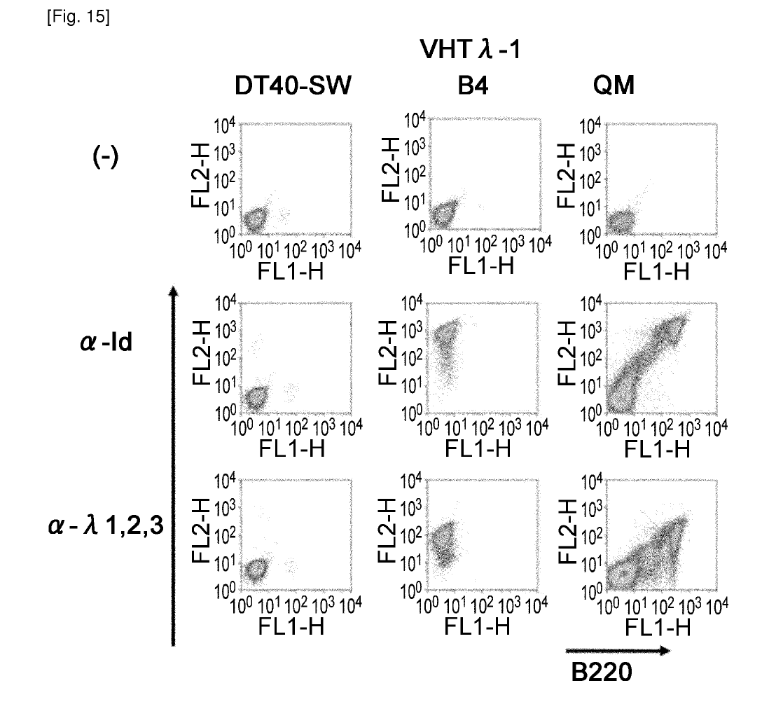

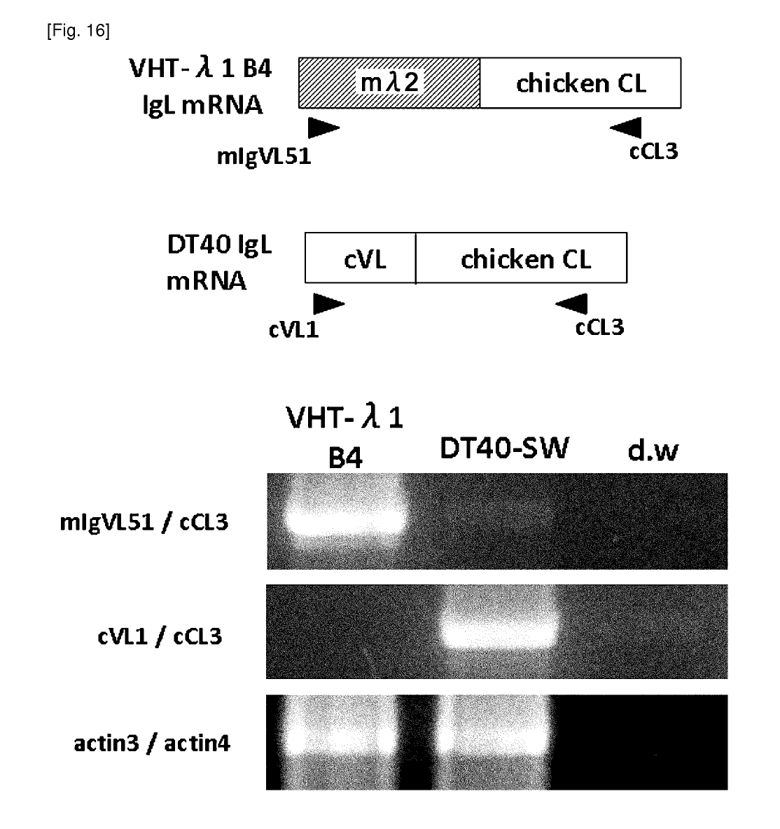

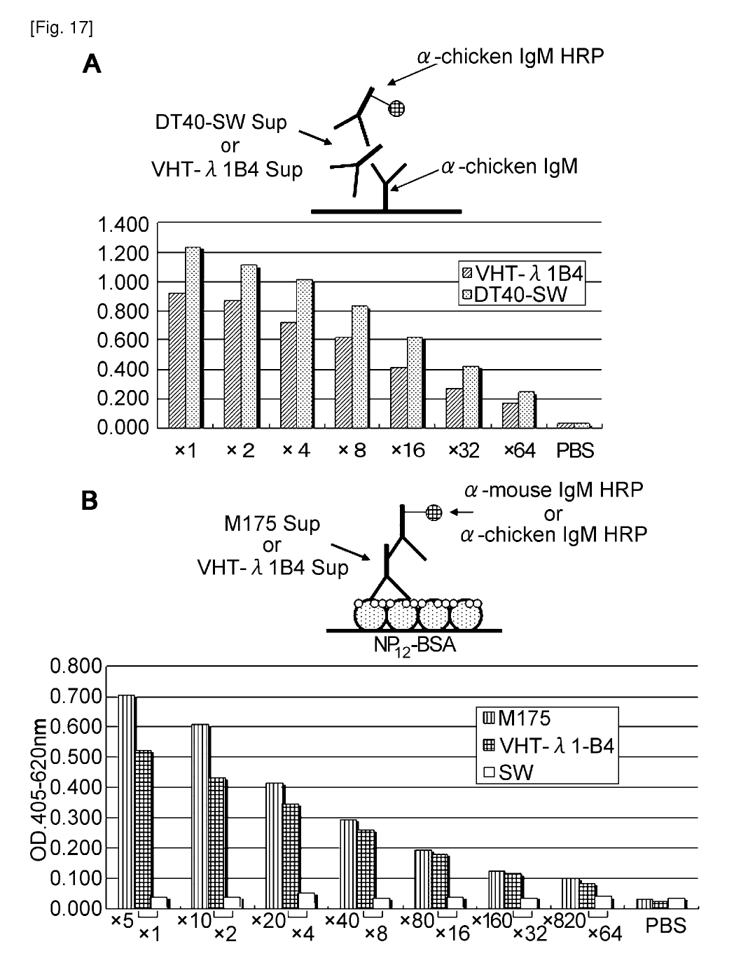

Of the cells prepared by introducing mouse lambda1 into C2 having mouse VHT, clone B4 was used in the subsequent experiments. As in the case of the heavy chain, when the cells were treated with 4-hydroxytamoxifen, cells with restored expression of the chicken IgM antibody on the cell surface were found by flow cytometry, and they lacked the drug resistance (FIG. 14). Furthermore, B4 in which expression of the antibody was switched on was subjected to limiting dilution, and expression of the mouse lambda1 chain variable region in the cells was assessed by flow cytometry. The cells were treated with a biotinylated rat monoclonal antibody (anti-Ig lambda1, lambda2, and lambda3 light chains; clone R26-46; BD Bioscience) which is expected to bind to the mouse lambda chain variable region, and then visualized with a phycoerythrin-labeled streptavidin. The result showed that the cells expressed the mouse lambda chain variable region (FIG. 15). The expression of VHT was also detected with an anti-idiotype antibody. Thus, it is considered that the introduced mouse antibody heavy chain and light chain were expressed on the cell surface in association with each other. RNA was isolated from each clone, and cDNA was prepared from the RNA. The splicing between the introduced variable region gene and the constant region gene was confirmed by PCR amplification and sequencing analysis of the amplified gene fragments. The production of mRNA of the chimeric light chain gene was assessed using the primers, mIgVL51 and cCL3, while the production of mRNA of the endogenous IgL gene was assessed using the primers, cVL1 and cCL3. The chimeric light-chain cDNA alone was amplified for clone B4. This result demonstrated that the splicing site attached to the 3′ end of the foreign variable region gene was functioning as designed, and thus the exons of the variable region gene and constant region gene were linked together as expected. (FIG. 16). Amplification of the beta-Actin gene using “actin3” (5′-CTGACTGACCGCGTTACTCCCACAGCCAGC-3′; SEQ ID NO: 35) and “actin4” (5′-TTCATGAGGTAGTCCGTCAGGTCACGGCCA-3′; SEQ ID NO: 36) was used as an internal standard. Sequencing analysis revealed that accurate splicing occurred at the splicing junctions. The culture supernatant of clone B4 into which mouse VHT/lambda1 was introduced was assayed for the secreted antibody using ELISA. 96-Well plates were coated with a goat anti-chicken IgM antibody (Bethyl). Using an HRP-labeled goat anti-chicken IgM antibody (Bethyl), the amount of antibody produced in the culture supernatant was compared to that of DT40-SW. The result showed that the cells produced the antibody at a level comparable to that of DT40-SW (FIG. 17A). Furthermore, NP was conjugated with bovine serum albumin by a previously reported method (Kanayama, N., et al., J. Immunol. 169, 6865-6874 (2002)), and 96-well plates were coated with this conjugate as an antigen. The antigen binding of antibodies in the culture supernatant was detected using an HRP-labeled goat anti-chicken IgM antibody. The culture supernatant of VHT/lambda1 antibody-producing clone B4 showed binding to the NP-conjugated antigen, similarly to the positive control in which the supernatant of a mouse anti-NP IgM antibody-producing hybridoma was analyzed using an HRP-labeled goat anti-mouse IgM antibody (Vector) (FIG. 17B). Meanwhile, the antibody produced by DT40-SW did not bind to the NP-conjugated antigen. Thus, it is demonstrated that clone B4 produces an NP-specific antibody. That is, it is shown that a foreign antibody heavy-chain variable region gene introduced by the above method can be normally transcribed and translated as a chimeric antibody with the chicken antibody light-chain constant region. Furthermore, the present inventors successfully prepared DT40 cells that express a chimeric antibody that retains the original function, in which both of the heavy chain and light chain variable regions are replaced with those of a mouse-derived foreign antibody.

(Introduction of Mutations into the Mouse Antibody Light Chain Variable Region)

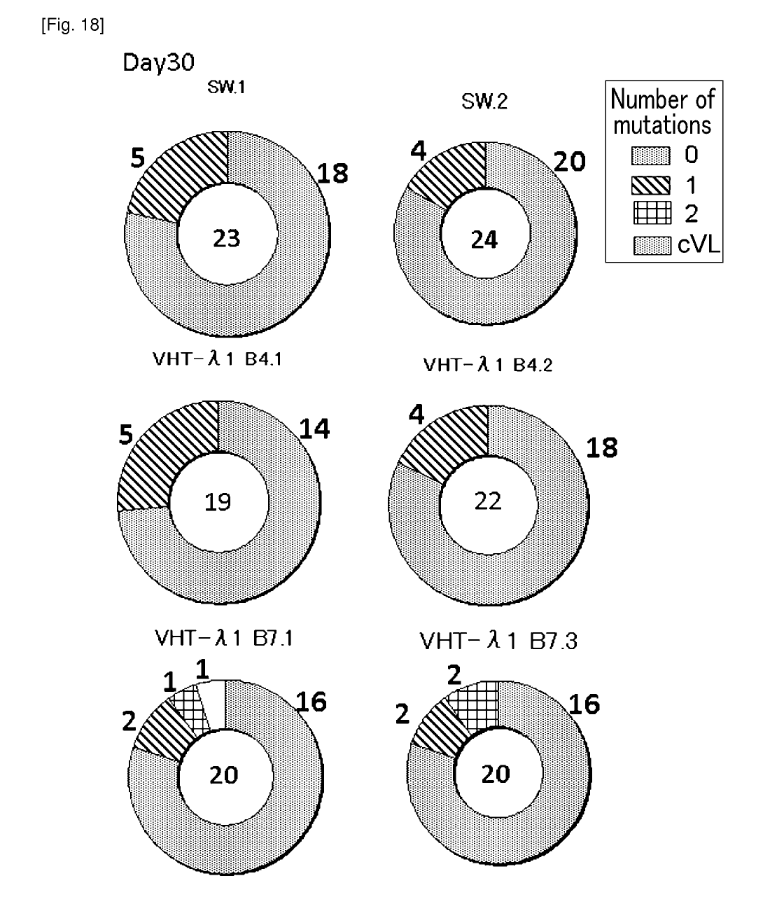

Cells of clones B4 and B7 expressing mouse VHT/lambda1 were treated with 4-hydroxytamoxifen. Then, GFP+ cells (i.e., cells whose AID expression which is essential for mutagenesis was switched on) were isolated as single cells by flow cytometry, and cultured for 30 days. Genomic DNA was isolated from the cultured cells. The light-chain variable region was amplified by PCR using the primers CVLF6 (SEQ ID NO: 37) and CVLR3 (SEQ ID NO: 38), and this was cloned into the pCR-Blunt vector and sequenced. Mutations were observed in clones B4 and B7 at a frequency comparable to that in wild-type DT40-SW (FIG. 18).

That is, it was proven that the above method can be used to introduce mutations and thus modify the variable region gene of an arbitrary foreign antibody introduced into the chicken light-chain variable region gene locus of DT40. The same effect can be produced by using VL targeting vector 2.

(5) Introduction of a Chicken Antibody Light Chain Variable Region

The antibody light chain variable region gene of DT40 was prepared as described below. cDNA was synthesized from total RNA of DT40-SW in which the mutation mechanism had never been switched on. The chicken antibody light-chain variable region (cVL) was amplified by PCR using the cDNA as a template, together with a sense primer

(cVL1: 5′-ACTCAGCCGTCCTCGGTGTCAGCAAACCCGGGA-3′; SEQ ID NO: 48), and an antisense primer

(cJL1: 5′-CGAGACTAGTTCAGCGACTCACCTAGGACGGTCAG-3′; SEQ ID NO: 49)

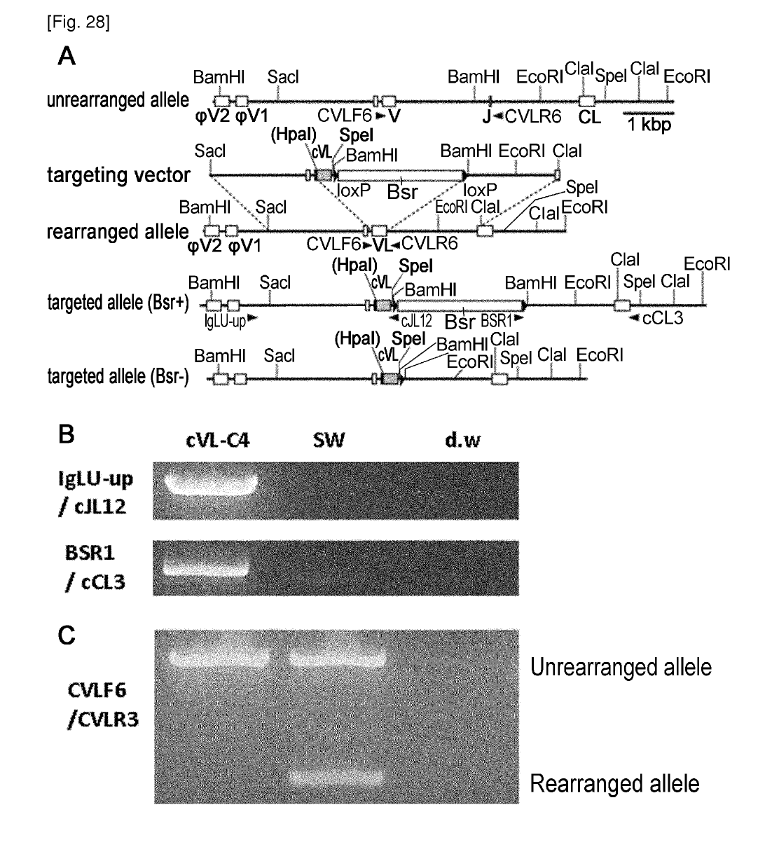

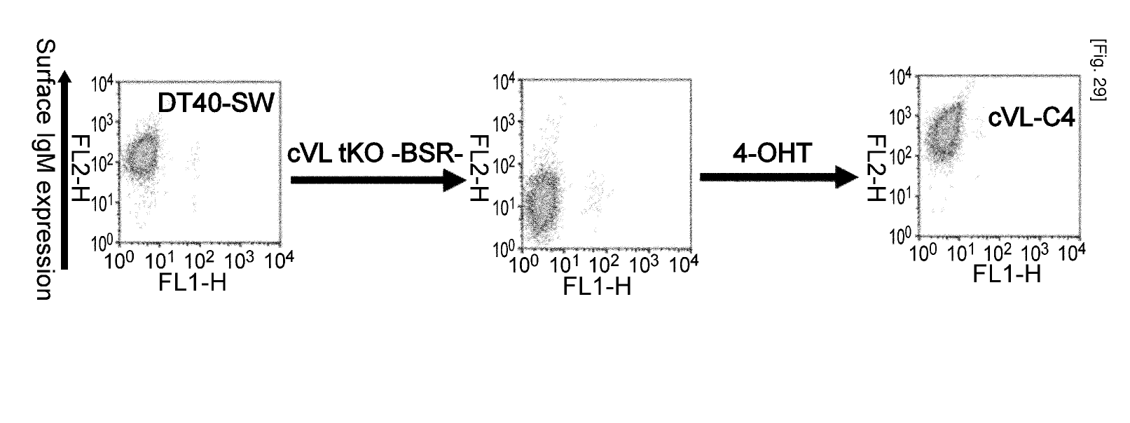

having a SpeI site. The resulting PCR fragment was digested with SpeI, and inserted into the HpaI-SpeI site of VL targeting vector 1 (FIG. 28A). The vector was linearized by SacI digestion, and introduced into DT40-SW cells by electroporation. After electroporation, selection was carried out using blasticidin S at a final concentration of 20 micro g/ml. Cells that formed colonies (54 clones) were stained with a phycoerythrin-labeled anti-chicken IgM mouse monoclonal antibody and analyzed using FACS Calibur. To obtain targeted cells, clones that became incapable of expressing the antibody heavy chain were selected by staining with an anti-chicken IgM antibody (3 clones). To assess these clones at the gene level, genomic DNA was extracted from the cells and tested for integration of the targeting vector by PCR using the primer pairs of IgLU-up and cJL12, and BSR1 and cCL3 (FIG. 28B). Furthermore, PCR was used to assess the targeting of the allele that resulted from VJ recombination using primers CVLF6 and CVLR3 (FIG. 28C). The result showed that targeting with the gene of interest was achieved in all of the clones.

Clone C2 was selected from them. As in the above case, when the clone was treated with 4-hydroxytamoxifen, cells with restored expression of the chicken IgM antibody on the cell surface were found by flow cytometry (FIG. 29).

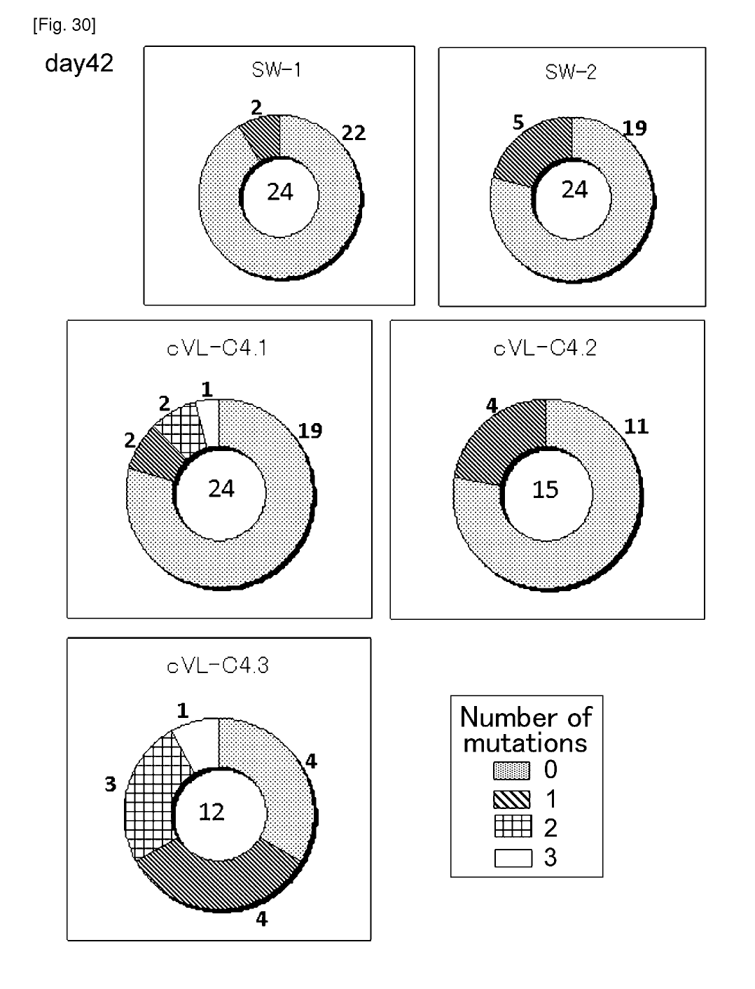

Furthermore, from the cells in which the antibody expression was switched on, GFP⁺ cells (i.e., cells whose AID expression which is essential for mutagenesis was switched on) were isolated as single cells by flow cytometry, and cultured for 30 days. Genomic DNA was isolated from the cultured cells. The light chain variable region was amplified by PCR using primers CVLF6 and CVLR3, and this was cloned into the pCR-Blunt vector and sequenced. Mutations were observed in the introduced chicken antibody light-chain variable region gene at a frequency comparable to that in wild-type DT40-SW (FIG. 30). That is, it was demonstrated that mutations are introduced into a chicken light-chain variable region gene introduced into DT40 by the above method at a frequency comparable to that in the wild-type gene.

### INDUSTRIAL APPLICABILITY

The present invention provides targeting vectors for introducing a DNA encoding a desired amino acid sequence into the antibody variable region gene locus of antibody-producing cells.

According to the present invention, desired polypeptides can be produced under specific conditions by introducing a DNA encoding a polypeptide comprising a desired amino acid sequence into the antibody variable region gene locus of antibody-producing cells.

Furthermore, polypeptides into which mutations are introduced can be produced using the ability of antibody-producing cells to introduce mutations.

The present invention is useful for functional modification of polypeptides.

1. A method of homologously recombining a DNA construct and a region comprising a DNA encoding an antibody variable region of an antibody-producing cell, which comprises the step of introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of a polypeptide comprising the desired amino acid sequence, and can be removed from the DNA construct.

2. The method of claim 1, wherein the DNA of (3) inhibits the production of a polypeptide comprising the desired amino acid sequence, and is located between two site-specific recombinase recognition sequences oriented in the same direction, and wherein the DNA of (3) comprises a promoter DNA and a marker gene that function in the cell, and the DNA of (3) can be removed from the DNA construct by a site-specific recombinase.

3. A method for selecting a cell, which comprises the steps of:
(a) allowing homologous recombination between a DNA construct and a region comprising a DNA encoding an antibody variable region of an antibody-producing cell, by introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of a polypeptide comprising the desired amino acid sequence, and can be removed from the DNA construct; and
(b) selecting a cell in which homologous recombination has occurred between the DNA construct and the region comprising a DNA encoding an antibody variable region of the antibody-producing cell.

4. The method of claim 3, wherein the DNA of (3) inhibits the production of a polypeptide comprising the desired amino acid sequence, and is located between two site-specific recombinase recognition sequences oriented in the same direction, and wherein the DNA of (3) comprises a promoter DNA and a marker gene that function in the cell, and the DNA of (3) can be removed from the DNA construct by a site-specific recombinase.

5. A method for producing an antibody-producing cell that produces a polypeptide, which comprises the steps of:
(a) allowing homologous recombination between a DNA construct and a region comprising a DNA encoding an antibody variable region of an antibody-producing cell, by introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of a polypeptide comprising the desired amino acid sequence, and can be removed from the DNA construct;
(b) selecting a cell in which homologous recombination has occurred between the DNA construct and the region comprising a DNA encoding an antibody variable region of the antibody-producing cell; and
(c) removing the DNA of (3) from the genomic DNA of the cell selected in step (b).

6. The method of claim 5, wherein the DNA of (3) inhibits the production of the polypeptide comprising the desired amino acid sequence and is located between two site-specific recombinase recognition sequences oriented in the same direction, and wherein the DNA of (3) comprises a promoter DNA and a marker gene that function in the cell, and the DNA of (3) can be removed from the DNA construct by a site-specific recombinase.

7. The method of claim 5, wherein the steps of (a) to (c) are defined as follows:
(a) allowing homologous recombination between a DNA construct and a region comprising a DNA encoding an antibody variable region of an antibody-producing cell, by introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;
(b) selecting a cell in which homologous recombination has occurred between the DNA construct and the region comprising a DNA encoding an antibody variable region of the antibody-producing cell; and
(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell.

8. The method of claim 5, wherein the produced polypeptide is displayed on the surface of the cell, and/or secreted to the outside of the cell.

9. The method of any one of claims 4, 6, and 7, wherein the cell is selected using the expression of the marker gene as an indicator.

10. The method of any one of claims 3, 4, 6, and 7, wherein the cell is selected using the absence of endogenous antibody expression in the antibody-producing cell as an indicator.

11. A method for producing an antibody-producing cell that produces a polypeptide into which a mutation is introduced, which comprises the steps of:
(a) allowing homologous recombination between a DNA construct and a region comprising a DNA encoding an antibody variable region of an antibody-producing cell expressing the AID gene, by introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of a polypeptide comprising the desired amino acid sequence, and can be removed from the DNA construct;
(b) selecting a cell in which homologous recombination has occurred between the DNA construct and the region comprising a DNA encoding an antibody variable region of the antibody-producing cell; and
(c) removing the DNA of (3) from the genomic DNA of the cell selected in step (b).

12. The method of claim 11, wherein the steps of (a) to (c) are defined as follows:
(a) allowing homologous recombination between a DNA construct and a region comprising a DNA encoding an antibody variable region of an antibody-producing cell expressing the AID gene, by introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of the polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase;
(b) selecting a cell in which homologous recombination has occurred between the DNA construct and the region comprising a DNA encoding an antibody variable region of the antibody-producing cell; and
(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell.

13. A method for producing an antibody-producing cell that produces a polypeptide into which a mutation is introduced, which comprises the steps of:
(a) allowing homologous recombination between a DNA construct and a region comprising a DNA encoding an antibody variable region of an antibody-producing cell in which the AID gene expression can be artificially switched on and off, by introducing into the antibody-producing cell a targeting vector comprising a DNA construct that comprises:
(1) a promoter DNA that functions in the cell;
(2) a DNA that encodes a desired amino acid sequence; and
(3) a DNA that inhibits the production of a polypeptide comprising the desired amino acid sequence, and can be removed from the DNA construct;
(b) selecting a cell in which homologous recombination has occurred between the DNA construct and the region comprising a DNA encoding an antibody variable region of the antibody-producing cell;
(c) removing the DNA of (3) from the genomic DNA of the cell selected in step (b);
(d) switching the AID gene expression on and off; and
(e) selecting a cell expressing the AID gene.

14. The method of claim 13, wherein the steps of (a) to (e) are defined as follows:
(a) allowing homologous recombination between a DNA construct and a region comprising a DNA encoding an antibody variable region of an antibody-producing cell, by introducing a targeting vector into the antibody-producing cell, wherein the targeting vector comprises a DNA construct comprising:
(1) a promoter DNA that functions in the cell;
(2) a DNA that encodes a desired amino acid sequence; and
(3) a DNA that inhibits the production of the polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase, wherein the endogenous AID gene in the antibody-producing cell is functionally destroyed, and the antibody-producing cell comprises a DNA construct comprising:
a promoter DNA that functions in the cell,
a DNA that is located between two site-specific recombinase recognition sequences oriented in opposite directions, and which can be inverted by a site-specific recombinase, and comprises an exogenous AID gene;
(b) selecting a cell in which homologous recombination has occurred between the DNA construct and the region comprising a DNA encoding an antibody variable region of the antibody-producing cell;
(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell;
(d) inverting the DNA located between the two site-specific recombinase recognition sequences oriented in opposite directions by reacting a site-specific recombinase at the site-specific recombinase recognition sequences in the genome of the cell selected in step (b), to switch the AID gene expression on and off; and
(e) selecting a cell expressing the AID gene.

15. The method of claim 13, wherein the steps of (a) to (e) are defined as follows:
(a) allowing homologous recombination between a DNA construct and a region comprising a DNA encoding an antibody variable region of an antibody-producing cell, by introducing a targeting vector into the antibody-producing cell,
wherein the endogenous AID gene in the antibody-producing cell is functionally destroyed and the antibody-producing cell comprises:
a DNA construct comprising
a promoter DNA that functions in the cell, and
a DNA that is located between two site-specific recombinase recognition sequences oriented in the opposite directions, and which can be inverted by a site-specific recombinase, and comprises an exogenous AID gene, and
a DNA construct comprising a promoter DNA that functions in the cell and a DNA encoding the site-specific recombinase,
wherein in the antibody-producing cell, the site-specific recombinase is activated in the presence of an extracellular stimulus and is not activated in the absence of the extracellular stimulus,
wherein the targeting vector comprises a DNA construct comprising:
(1) a promoter DNA that functions in the cell;
(2) a DNA that encodes a desired amino acid sequence; and
(3) a DNA that inhibits the production of a polypeptide comprising the desired amino acid sequence, and is located between two site-specific recombinase recognition sequences oriented in the same orientation, and which comprises a promoter DNA and a marker gene that function in the cell, and can be removed from the DNA construct by the site-specific recombinase;
(b) selecting a cell in which homologous recombination has occurred between the DNA construct and the region comprising a DNA encoding an antibody variable region of the antibody-producing cell;
(c) removing the DNA located between two site-specific recombinase recognition sequences oriented in the same direction from the genomic DNA of the cell selected in step (b), by reacting a site-specific recombinase at the site-specific recombinase recognition sequences in the genome of the cell via activation of the site-specific recombinase activity by an extracellular stimulus to the cell;
(d) inverting the DNA located between the two site-specific recombinase recognition sequences oriented in opposite directions, by reacting a site-specific recombinase at the site-specific recombinase recognition sequences in the genome of the cell selected in step (b), via activation of the site-specific recombinase activity by an extracellular stimulus to the cell, to switch the AID gene expression on and off; and
(e) selecting a cell expressing the AID gene.

16. The method of claim 11 or claim 13, wherein the produced polypeptide into which a mutation is introduced is displayed on the surface of the cell and/or secreted to the outside of the cell.

17. The method of claim 11 or claim 13, wherein the cell is selected in step (b) using the expression of the marker gene as an indicator.

18. The method of claim 11 or claim 13, wherein the cell is selected in step (b) using the absence of endogenous antibody expression in the antibody-producing cell as an indicator.

19. The method of claim 15, wherein the site-specific recombinase is a fusion protein of a site-specific recombinase with an estrogen receptor or a protein comprising an estrogen-binding domain thereof; and wherein a ligand capable of binding to the estrogen-binding domain serves as the extracellular stimulus.

20. The method of claim 19, wherein the estrogen receptor or estrogen-binding domain thereof is a mouse mutant estrogen receptor in which glycine is substituted with arginine at amino acid position 525, or a mouse mutant estrogen-binding domain thereof; and wherein the ligand capable of binding to the estrogen-binding domain is 4-hydroxytamoxifen.

21. (canceled)

22. (canceled)

23. The method of claim 11 or claim 13, wherein the antibody-producing cell is a B cell.

24. The method of claim 23, wherein the B cell is derived from chicken.

25. The method of any one of claims 2, 4, 6, 7, 12, 14, and 15, wherein the combination of site-specific recombinase and site-specific recombinase recognition sequence is (i) or (ii) below:
(i) the site-specific recombinase is a Cre recombinase, and the site-specific recombinase recognition sequence is a loxP sequence;
(ii) the site-specific recombinase is an FLP recombinase, and the site-specific recombinase recognition sequence is an FRT sequence.

26. The method of claim 1, wherein the polypeptide comprising a desired amino acid sequence comprises an amino acid sequence of an antibody constant region and a desired amino acid sequence.

27. The method of claim 26, wherein the desired amino acid sequence is an amino acid sequence of an antibody variable region.

28. The method of claim 1, wherein the polypeptide comprising a desired amino acid sequence is an antibody heavy or light chain.

29. A method for producing a DNA encoding a polypeptide or a polypeptide into which a mutation is introduced, which comprises the steps of:
(a) producing an antibody-producing cell that produces a polypeptide or a polypeptide into which a mutation is introduced, by the method of claim 5 or claim 11; and
(b) isolating a DNA encoding the polypeptide or polypeptide into which a mutation is introduced from the antibody-producing cell produced in step (a).

30. A method for producing a polypeptide or a polypeptide into which a mutation is introduced, which comprises the steps of:
(a) producing an antibody-producing cell that produces a polypeptide or a polypeptide into which a mutation is introduced, by the method of claim 5 or claim 11; and
(b) isolating the polypeptide or polypeptide into which a mutation is introduced from the antibody-producing cell produced in step (a) or a secreted product from the cell.

31. A method for producing a polypeptide or a polypeptide into which a mutation is introduced, which comprises the steps of:
(a) producing a DNA encoding a polypeptide or a polypeptide into which a mutation is introduced by the method of claim 29; and
(b) isolating the polypeptide encoded by the DNA produced in step (a).

32. An antibody-producing cell in which a region comprising a DNA encoding an antibody variable region is homologously recombined with a DNA construct comprising:
(1) a promoter DNA that functions in the cell;
(2) a DNA that encodes a desired amino acid sequence; and
(3) a DNA that inhibits the production of a polypeptide comprising the desired amino acid sequence and can be removed from the DNA construct.

33. The cell of claim 32, wherein the DNA of (3) is a DNA that inhibits the production of a polypeptide comprising the desired amino acid sequence and is located between two site-specific recombinase recognition sequences oriented in the same direction, and which comprises a promoter DNA and a marker gene that function in the cell, and can be removed from the DNA construct by a site-specific recombinase.

34. The cell of claim 32, wherein the antibody-producing cell expresses the AID gene.

35. The cell of claim 32, wherein the antibody-producing cell is a cell in which the AID gene expression can be artificially switched on and off.

36. The cell of claim 35, wherein the endogenous AID gene is functionally destroyed, and the cell comprises a DNA construct that comprises:
a promoter DNA that functions in the cell; and
a DNA that is located between two site-specific recombinase recognition sequences oriented in opposite directions, and which can be inverted by a site-specific recombinase, and comprises an exogenous AID gene.

37. The cell of claim 36, wherein the antibody-producing cell further comprises a DNA construct comprising a promoter DNA that functions in the cell and a DNA encoding a site-specific recombinase, wherein the site-specific recombinase is activated in the presence of an extracellular stimulus and is not activated in the absence of the extracellular stimulus.

38. The cell of claim 37, wherein the site-specific recombinase is a fusion protein of a site-specific recombinase with an estrogen receptor or a protein comprising an estrogen-binding domain thereof.

39. The cell of claim 38, wherein the estrogen receptor or estrogen-binding domain thereof is a mouse mutant estrogen receptor in which glycine is substituted with arginine at amino acid position 525, or a mouse mutant estrogen-binding domain thereof.

40. (canceled)

41. (canceled)

42. The cell of claim 32, wherein the antibody-producing cell is a B cell.

43. The cell of claim 42, wherein the B cell is derived from chicken.

44. The cell of claim 33, wherein the combination of site-specific recombinase and site-specific recombinase recognition sequence is (i) or (ii) below:
(i) the site-specific recombinase is a Cre recombinase, and the site-specific recombinase recognition sequence is a loxP sequence;
(ii) the site-specific recombinase is an FLP recombinase, and the site-specific recombinase recognition sequence is an FRT sequence.

45. The cell of claim 32, wherein the polypeptide comprising a desired amino acid sequence comprises an amino acid sequence of an antibody constant region and a desired amino acid sequence.

46. The cell of claim 45, wherein the desired amino acid sequence is an amino acid sequence of an antibody variable region.

47. The cell of claim 32, wherein the polypeptide comprising a desired amino acid sequence is an antibody heavy or light chain.

48. A kit comprising the cell of claim 32.

49. A gene targeting vector comprising a DNA construct that comprises:
(1) a promoter DNA that functions in the cell;
(2) a DNA that comprises a cloning site; and
(3) a DNA that can be removed from the DNA construct and inhibits the production of a polypeptide comprising a desired amino acid sequence encoded by a DNA inserted into the DNA of (2);
wherein the gene targeting vector is used for homologously recombining the DNA construct and a region comprising a DNA encoding an antibody variable region of an antibody-producing cell.

50. The vector of claim 49, wherein the DNA of (3) is a DNA that can be removed from the DNA construct by a site-specific recombinase, and inhibits the production of a polypeptide comprising the desired amino acid sequence encoded by the DNA inserted into the DNA of (2), and which is located between two site-specific recombinase recognition sequences oriented in the same orientation, and comprises a promoter DNA and a marker gene that function in the cell.

51. The vector of claim 49, in which a DNA encoding a desired amino acid sequence is inserted into the cloning site.

52. The vector of claim 49, wherein the polypeptide comprising a desired amino acid sequence comprises an amino acid sequence of an antibody constant region and a desired amino acid sequence.

53. The vector of claim 52, wherein the desired amino acid sequence is an amino acid sequence of an antibody variable region.

54. The vector of claim 49, wherein the polypeptide comprising a desired amino acid sequence is an antibody heavy or light chain.

55. A cell comprising the targeting vector of claim 49.

56. A kit comprising the targeting vector of claim 49.

57. A DNA comprising the nucleotide sequence of SEQ ID NO: 7.

58. A vector comprising the DNA of claim 57.

59. A cell comprising the DNA of claim 57 or the vector of claim 58.


SEQUENCE LISTING (53 sequences)

SEQ ID NO: 1 - DNA, 29 bp, Artificial
  an artificially synthesized sequence
ggggtacccggaggagacgatgacttcgg

SEQ ID NO: 2 - DNA, 28 bp, Artificial
  an artificially synthesized sequence
cttcggtcccgtggccccatgcgtcgat

SEQ ID NO: 3 - DNA, 20 bp, Artificial
  an artificially synthesized sequence
ggttcaatgtagtccagtcc

SEQ ID NO: 4 - DNA, 464 bp, Gallus gallus
gaagaagcacaaagtggccattaaataattaattgcacccaattaattcctgatggggattactgcagaggaactcagcgaaggattcaagcagaaatgacacagttaggttgctcaattgcgttaattaattaaaatcgctaattaggtgagcttcattaaggcctgatggggttcatcccaacgctgggatcaaagaactcagcgcagagtggataacgataattagaaggggacaaattaatgaggaaacacgactttgggcctagacggccattaaatcattaatcaacccgacgggcgcccaattaatcataaagttattaagcaattaagtcgaggctgacgagaaaccgcaacgagagccctccacgctcctttgagttggcgcaacaacaacaacgcgggctcgttaagcagcgacgacgccgagcgtaacgaagcatccccgcgcccgacaatta

SEQ ID NO: 5 - DNA, 26 bp, Artificial
  an artificially synthesized primer sequence
gtggccattctagaattaattgcacc

SEQ ID NO: 6 - DNA, 22 bp, Artificial
  an artificially synthesized primer sequence
ggagggatccggcttcgttagc

SEQ ID NO: 7 - DNA, 7931 bp, Gallus gallus
tctagatgacagctctactgtcactgcttcaggcttcataagcacagggatgaaagtcttacctagattggtgggagagcttgtacaattattaactgatacacaggtacattacatcctatatatagtcttgtacattcatagtaagtaaagactgaactaaatttagaaaggtacccaggatacaatttaagcaggggaaaataatttaaagaaaaaccaacaacaacaacactccagatagatgttagtcttgtcttaaaataccaaaagcagtaaggttttcaaaattcgtcatgttttcagaagcgacatttcattctggtcccaatactcattttaaaatactctgtactcacaacagtaacaggatgttccatatggaagtcaatagcttaattgctactgtcaatgaggtacctatgataccacctcaaaaataggagcaaccaccgtgtcctgaggtcctgtgcagcagaatctgccacaattctgactgaagtcaccacacctcagctgtgcagccagacccccagaatatctgttaggaaaaatatcactagaaataataatctctttagtcaaagtggagcataaggggaagaaatgtaatctatcacagtgagagcttgatcagagaaatatggaaatatgcagactggtatgaatggagcatggatggaaatatctgtttgggaaagcaccgaaacgtactgcacagaaatcacttggattacatgtattgatatccaaggctatgactgacatcatccttctatcagttgtgtaaataatgtgttaggagagatatgatcttgttaaaatcttgaattaaggaagcagatgccaaaattaaactttgcataaagacatctgcctagttgtactaaacattcttccattttctggaggaatgaaataaaaaatcaaccaggaggaaagttcacttcttgcaaagactgacactcgagtgtctcaggtactaatgaaacattaaagaaattccatttacattcattacaacacagttttcctgcactgagctcagacacagctaagtcctctcttgcatgaaatccaggtttatctgcaaaaaagattacatagtgaaagtcaaagtctttgtcttgtatataaggtcatggaccaaaaaggagcatgttgagctatactgaaggtgacaatatactgaatgccacagatcactaagaggtcccagaatcacagatcccaagaagtcacagaaatccaaccaagaccatatgatgttctacaggaagaaagtaggatagacatagtgaaaaagcatatttgtctataaatgatatataatttctgatttatagtttccaatttatggaacagtgtaagacttcagtatcaaagatgacactaactttttttttttaaaaaaaaaaagggcaaggaaagtaagggaatgtatacaaagccacttagtgtccttccactgacaggtgatgatgtatggttgtaggtgtaggaccagacatgcttgatggctctttccagatgtagaagacagtgacttcatgggataacataaagattaaagcaaaaatggggtgattttccctaaagtactgtcttattttctgagaagcatgcatcagggtcttcctgaggtacatattgcatccatggcattattttcaacagccatcaattgatctcttcttccattggcaggtagtaacttttgaaagctctctgtcttgattacttacaattatctaaaatgtttgtgtcatttagtcttctatgatcatctgcatatatttccctttgcacattcaccgacatttaagtgggatccctaattaatgttggcgcagaagggattcatatcaattaattccctgtcctcaattaatgcataattgggagtgcgatggaatcattcaataagtaatgaggacttggtgtcaaaatgccaattaagtaattaatgacacccaattaatacacaatggggattacagtggaataactcagtgaagttcatatctaaattgcaattgaataattaattacagctaattaatggatgggagactggcagtggtataacttaatgaaggaacacagtgaattcctgtctgaattgaaattaattgatttattagggctaattaatgaatggtggggttaaaaatagcattactcagcgaaggaacatgagttcatggctaaattgagattgaattattaattacaactaattgattaatgactgatgggagtaagaatggaataactcagtgaaggaacacaatggattcatggcctaattgaaattaaatggttaattagagctaattaatgaatgatggggggtggcaatggaatgatacaagaaagaaacacaatgggttcgtgtctacattgaaattaaataattaattacagctaattaataaacggcgaggttgacaatggtataactgtgaagagacagaatgagttcgtgactaaattggtattaaatgattaattagagctaattaacaggtggtggggctgagaatggcataactcaatgggggatgatgagctgatggcttaattggaattaataattaatcacaaccaattaacaggtgctgttaatggggttacaggggcatgacttcaggaagaagcacaaagtggccattaaataattaattgcacccaattaattcctgatggggattactgcagaggaactcagcgaaggattcaagcagaaatgacacagttaggttgctcaattgcgttaattaattaaaatcgctaattaggtgagcttcattaaggcctgatggggttcatcccaacgctgggatcaaagaactcagcgcagagtggataacgataattagaaggggacaaattaatgaggaaacacgactttgggcctagacggccattaaatcattaatcaacccgacgggcgcccaattaatcataaagttattaagcaattaagtcgaggctgacgagaaaccgcaacgagagccctccacgctcctttgagttggcgcaaacaacaaacaacgcgggctcgttaagcagcgacgacgccgagcgtaacgaagcaatccccgcgcccgacaattaacgcgctaacgaagccggagccctccttatgcaaattagcccctccagagggccataaaagcgccggctctccgacggaggagcaccagtcggctccgcaaccatgagcccactcgtctcctccctcctgctcctggccgccctgccaggtgagggcgctgtggggctctatggggctctatggggtctcagcggggctctgcgggctcaatgggggccaaagggggggtctgcgggctctatgggggggtcaacggggggtctcacggggggccggctccgcgaggccgtgtggcggcggctccgtcagcgctctctgcccttccccacagggctgatggcggccgtgacgttggacgagtccgggggcggcctccagacgcccgggggagcgctcagcctcgtctgcaaggcctccgggttcaccttcagcagtaacgccatgggttgggtgcgacaggcgcccggcaaggggctggagtgggtcgctggtattgatgatgatggtagtggcacaagatacgcgccggcggtgaagggccgtgccaccatctcgagggacaacgggcagagcacactgaggctgcagctgaacaacctcagggctgaggacaccggcatctactactgcacgaaatgtgcttacagtagtggttgtgattatgaagctggttatatcgacgcatggggccacgggaccgaagtcatcgtctcctccggtgagtcttcaaccccccccaaaattgccgcggcgattttggggcaaaatcgggcgattttgggtcagtcgaagggggcggtcggtccatcatttgcggccgggtgatttttggggccgaaaagtgggaatttggggcccaatttggggcccaatttggggcccaatttgggttttcgaggggggatttttttagggggagattttgggtccccggaaaatggtgattttgggtcaaaaatggggattttggggtcgttttgaggtggggttttttggggtagaaatggcggtctgaggggaaaatgtcgttttggggccattttgggccatttgaggggaaatttgggtcaaaaaatgacgattttgggtcgttttagggaggaaaatgaatttagggcgatttgagccaaatttgggtcaaaaatggcgattttgagtcaaaaaatgattattttgggtcattttagggaggaaaatgaatttagggagattggacggcaaatttgggtcaaaaaatggtgattttgggtcaaaaaatgattattttgggtcattttagggagaaaaatgaatttagggagatttgagggcaaatttgggtcaaaaaatggtgattttgggtcaaaaaatgattattttgggtcattttagggagaaaaatgaatttagggagatttgagggcaaatttgggtcgaaaaatggcgattttgggtgaaaaatggacaattttgggtcattttagggttaaaaatgaatttagggagattggacggcaaatttgggtcaaaaaatggtgatttggggtcaaaaaataattattttgggtcattttagggataaaaaatgaatttagggagatttgagggcaaatttgggtcgaaaaatggtgatttggggtgaaaaatggacaattttgggtcattttagggataaaaaatgaatttagggcgatttgagccaaatttgggtcaaaaatggcgattttgagtcaaaaaatgattattttgggtcattttagggttaaaaatgaatttagggagattggacggcaaatttgggtcaaaaaatggtgattttcggtcaaaaaatgattattttgggtcattttagggagaaaaatgaatttagggagattggacggcaaatttgggtcgaaaaatggtgattttgggtcaaaaatggacaatttggggtcattttagggataaaaatgaatttagggagatttgagagcaaatttgggtctaaaaatgtcaatttggggtcaaaaatggtgattttgggtcattttagggatataaatgaattttgggagatttgagggcaaatttgggtcgaaaaatggtgatttggggtcaatttagggaggaaaatgaatttaaggtgatttgacggcaaatttgggttgaaaaatgacaatttggggtcattttaaagataaaatgaatttagggcgatttgagggcaaatttgggtcgaagaatggtgatttgggtcaaaaaatgacaattttgggtcattttagggatataaatggacttagagcgatttgagggcaaatttggttgaaaaaatggacaatttgggtcattttagggatataaatgaatttagggagatttgatggcaaatttgggtcgaaaaatggtgatttgggtcaaaaatggtgattttggttgaaaaacagtcatttggggtcattttagggattaaaatgaatttcgggagatttgagggcaaatttgggtcaaaaaatggtgattttgggtcaattagtgataaaaaaatgaatttagggtgatttgagggcaaatttgggtcaaaaaatgacaatttggggtttttttagggaggagaatgaatttagggagttttgagggcaaatttgggtcaaaaaatagtgatttgggtcaaaagtggtgattttggttgaaaaacagtcattttgggtcattttagggagaaaaatgaatttcgggagatttgagggcaaatttgggtcaaaaaatgacaattttgggtcactttacgaattaaaatgaatttagggagatttgagggcaaatttgggtcaaaatatggtgattttgggtcaatttagggaggaaaatgaatttagggcgatttgaaggcaaatttgggtcgaaaatggtgattttgggtcaaaaaatgatgattttgggtcgttttagggagaaaatgaatttagggagatttgagggcaaatttgggtcaaaaaatggtgattttgggtcattgtagggataaaaatgaatttggggcgatttgagggcaaatttgggtggaaaatggacaattttgggtcattttagggagaaaaatgaatttagggagatttgagggcaaatttgggtcaaaaaatggtgatttggggtcattttcagaagaaaaaatgattattttccccactaaaaatgtatattttggggccaaatggtgaaaaatggtgatttttaatcaaacgtccccaaaattggggaaatttcatcgatttgacccaaaattgagttttttttccctgttaaaaatgtacattttggggtcaatcgctgaaatgttcccacttttcacttctttgcccccaaattttgctgtccggtgagaaattaattaattaatgatcctaaaggcagagtgctgtcaacattcctaattggttattacccaataacacacaccctcgagtgtcttgggactacaaaacactttttacgtagccttcctacatgaataatgaaaacttcaaatattcatccaattatattcagcttctctcttgactaagggttgccaaatgcaattggacatacttctgacatagaatttcaggctgtatctgtgttggaagggcttagtcataattcttgacttctggggagagattagataacaccgtgatggtaatttctccagcctgaaaacctattgtttattcacttttgctattaaattctggtttagtagcagtataattgcagaatttaaaaagtctattttttaattttatactaggagcagcacaggttccatttgaagcaagaagacaatttgcttgttctccaagtgctgctgatacagctgcatgactgaagtcaccggcaagcatttgaaagccagatggttattgttcatagagctattttgtgttggttttcctctctaatcattttgctgtcaatcatgattggctcccaatgtcttattttaaaacgaattttttttaagtcttgctaacatgctgttataggtggaaataggctgagcaatggtagcaggaaagatgcctccctgcttgctttcactgtgtaaggccgaactcatggaaaaggatgaagagaaaagctgctgcttttcagtagctaatgcctatttggaggctctgctttttgtcaggcattctcatttttccagcttctaatattgttgtctcataaagccgggccctgggccatgcctttcatactggcctttccctgcttagtagctaacccagataaactcatcaacagagctacaattgcgctttcccctttcccctttggattaatggcgcagctccccgtgactgagtggatgctgctctctgagctgcagaggtctggttccaggtgagcctctaattcttcactgcagacacagaaataacagcccaaatcaggtgtgtttcatgtgtctctttggtgggcttaacaaaaggagaggtgaaaaggatgtgtgtcaagcgttggtttgaaaatagaagcagctggtagggctgcaactttccattgcagttagcctccatctaggtcaccttattacctatgaaaatgtgaagctatgtaagaggtgtgagtccttccttcttttttctaatagagtcctgaaaacatgaaaggtcataggaaatgatagagaacacattccaggcacctgtaccgtgccaaaatcacaaagctggtgggacggtttccatttgtagaatcaaattggccgtgttgtttccagtcccatcccaagctgcgggagggccgcagcactgtgtgatccgaattcttcgccctatagtgagtcgtattacgcggccgc

SEQ ID NO: 8 - DNA, 25 bp, Artificial
  an artificially synthesized primer sequence
gtgggatccctaattaatgttggcg

SEQ ID NO: 9 - DNA, 52 bp, Artificial
  an artificially synthesized primer sequence
tattccgcggactagtacgtcagctgaacctccgccatcagccctgtgggga

SEQ ID NO: 10 - DNA, 7282 bp, Artificial
  an artificially synthesized sequence
ggatccctaattaatgttggcgcagaagggattcatatcaattaattccctgtcctcaattaatgcataattgggagtgcgatggaatcattcaataagtaatgaggacttggtgtcaaaatgccaattaagtaattaatgacacccaattaatacacaatggggattacagtggaataactcagtgaagttcatatctaaattgcaattgaataattaattacagctaattaatggatgggagactggcagtggtataacttaatgaaggaacacagtgaattcctgtctgaattgaaattaattgatttattagggctaattaatgaatggtggggttaaaaatagcattactcagcgaaggaacatgagttcatggctaaattgagattgaattattaattacaactaattgattaatgactgatgggagtaagaatggaataactcagtgaaggaacacaatggattcatggcctaattgaaattaaatggttaattagagctaattaatgaatgatggggggtggcaatggaatgatacaagaaagaaacacaatgggttcgtgtctacattgaaattaaataattaattacagctaattaataaacggcgaggttgacaatggtataactgtgaagagacagaatgagttcgtgactaaattggtattaaatgattaattagagctaattaacaggtggtggggctgagaatggcataactcaatgggggatgatgagctgatggcttaattggaattaataattaatcacaaccaattaacaggtgctgttaatggggttacaggggcatgacttcaggaagaagcacaaagtggccattaaataattaattgcacccaattaattcctgatggggattactgcagaggaactcagcgaaggattcaagcagaaatgacacagttaggttgctcaattgcgttaattaattaaaatcgctaattaggtgagcttcattaaggcctgatggggttcatcccaacgctgggatcaaagaactcagcgcagagtggataacgataattagaaggggacaaattaatgaggaaacacgactttgggcctagacggccattaaatcattaatcaacccgacgggcgcccaattaatcataaagttattaagcaattaagtcgaggctgacgagaaaccgcaacgagagccctccacgctcctttgagttggcgcaaacaacaaacaacgcgggctcgttaagcagcgacgacgccgagcgtaacgaagcaatccccgcgcccgacaattaacgcgctaacgaagccggagccctccttatgcaaattagcccctccagagggccataaaagcgccggctctccgacggaggagcaccagtcggctccgcaaccatgagcccactcgtctcctccctcctgctcctggccgccctgccaggtgagggcgctgtggggctctatggggctctatggggtctcagcggggctctgcgggctcaatgggggccaaagggggggtctgcgggctctatgggggggtcaacggggggtctcacggggggccggctccgcgaggccgtgtggcggcggctccgtcagcgctctctgcccttccccacagggctgatggcggccgtgcagctgacgtactagtccgccagatcccctaccgttcgtataatgtatgctatacgaagttatagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtcacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagccaagcttggtatggtaaaacttttaatttcgggtatatttgagtggaatgagttcttcaatcgtagttttgactaacttgccattcatttctattaacacaaaacaatctggtgcatagtctgaaatcaactccctacacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcttgttgagaaatgttaaatgttttcatatattcttcttcctccgatttctgcaaaaatgaatccaagcttgaattaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgaggatctgggctagccctgatcaataacttcgtataatgtatgctatacgaacggtaggggatctggcggcgattttggggcaaaatcgggcgattttgggtcagtcgaagggggcggtcggtccatcatttgcggccgggtgatttttggggccgaaaagtgggaatttggggcccaatttggggcccaatttggggcccaatttgggttttcgaggggggatttttttagggggagattttgggtccccggaaaatggtgattttgggtcaaaaatggggattttggggtcgttttgaggtggggttttttggggtagaaatggcggtctgaggggaaaatgtcgttttggggccattttgggccatttgaggggaaatttgggtcaaaaaatgacgattttgggtcgttttagggaggaaaatgaatttagggcgatttgagccaaatttgggtcaaaaatggcgattttgagtcaaaaaatgattattttgggtcattttagggaggaaaatgaatttagggagattggacggcaaatttgggtcaaaaaatggtgattttgggtcaaaaaatgattattttgggtcattttagggagaaaaatgaatttagggagatttgagggcaaatttgggtcaaaaaatggtgattttgggtcaaaaaatgattattttgggtcattttagggagaaaaatgaatttagggagatttgagggcaaatttgggtcgaaaaatggcgattttgggtgaaaaatggacaattttgggtcattttagggttaaaaatgaatttagggagattggacggcaaatttgggtcaaaaaatggtgatttggggtcaaaaaataattattttgggtcattttagggataaaaaatgaatttagggagatttgagggcaaatttgggtcgaaaaatggtgatttggggtgaaaaatggacaattttgggtcattttagggataaaaaatgaatttagggcgatttgagccaaatttgggtcaaaaatggcgattttgagtcaaaaaatgattattttgggtcattttagggttaaaaatgaatttagggagattggacggcaaatttgggtcaaaaaatggtgattttcggtcaaaaaatgattattttgggtcattttagggagaaaaatgaatttagggagattggacggcaaatttgggtcgaaaaatggtgattttgggtcaaaaatggacaatttggggtcattttagggataaaaatgaatttagggagatttgagagcaaatttgggtctaaaaatgtcaatttggggtcaaaaatggtgattttgggtcattttagggatataaatgaattttgggagatttgagggcaaatttgggtcgaaaaatggtgatttggggtcaatttagggaggaaaatgaatttaaggtgatttgacggcaaatttgggttgaaaaatgacaatttggggtcattttaaagataaaatgaatttagggcgatttgagggcaaatttgggtcgaagaatggtgatttgggtcaaaaaatgacaattttgggtcattttagggatataaatggacttagagcgatttgagggcaaatttggttgaaaaaatggacaatttgggtcattttagggatataaatgaatttagggagatttgatggcaaatttgggtcgaaaaatggtgatttgggtcaaaaatggtgattttggttgaaaaacagtcatttggggtcattttagggattaaaatgaatttcgggagatttgagggcaaatttgggtcaaaaaatggtgattttgggtcaattagtgataaaaaaatgaatttagggtgatttgagggcaaatttgggtcaaaaaatgacaatttggggtttttttagggaggagaatgaatttagggagttttgagggcaaatttgggtcaaaaaatagtgatttgggtcaaaagtggtgattttggttgaaaaacagtcattttgggtcattttagggagaaaaatgaatttcgggagatttgagggcaaatttgggtcaaaaaatgacaattttgggtcactttacgaattaaaatgaatttagggagatttgagggcaaatttgggtcaaaatatggtgattttgggtcaatttagggaggaaaatgaatttagggcgatttgaaggcaaatttgggtcgaaaatggtgattttgggtcaaaaaatgatgattttgggtcgttttagggagaaaatgaatttagggagatttgagggcaaatttgggtcaaaaaatggtgattttgggtcattgtagggataaaaatgaatttggggcgatttgagggcaaatttgggtggaaaatggacaattttgggtcattttagggagaaaaatgaatttagggagatttgagggcaaatttgggtcaaaaaatggtgatttggggtcattttcagaagaaaaaatgattattttccccactaaaaatgtatattttggggccaaatggtgaaaaatggtgatttttaatcaaacgtccccaaaattggggaaatttcatcgatttgacccaaaattgagttttttttccctgttaaaaatgtacattttggggtcaatcgctgaaatgttcccacttttcacttctttgcccccaaattttgctgtccggtgagaaattaattaattaatgatcctaaaggcagagtgctgtcaacattcctaattggttattacccaataacacacaccctcgaggccgc

SEQ ID NO: 11 - DNA, 38 bp, Artificial
  an artificially synthesized primer sequence
ttagaaggggacaaattaatgaggaaacacgactttgg

SEQ ID NO: 12 - DNA, 47 bp, Artificial
  an artificially synthesized sequence
cgactagtccgcatgcagccctgtggggaagggcagagagcgctgac

SEQ ID NO: 13 - DNA, 7271 bp, Artificial
  an artificially synthesized sequence
tctaggatccctaattaatgttggcgcagaagggattcatatcaattaattccctgtcctcaattaatgcataattgggagtgcgatggaatcattcaataagtaatgaggacttggtgtcaaaatgccaattaagtaattaatgacacccaattaatacacaatggggattacagtggaataactcagtgaagttcatatctaaattgcaattgaataattaattacagctaattaatggatgggagactggcagtggtataacttaatgaaggaacacagtgaattcctgtctgaattgaaattaattgatttattagggctaattaatgaatggtggggttaaaaatagcattactcagcgaaggaacatgagttcatggctaaattgagattgaattattaattacaactaattgattaatgactgatgggagtaagaatggaataactcagtgaaggaacacaatggattcatggcctaattgaaattaaatggttaattagagctaattaatgaatgatggggggtggcaatggaatgatacaagaaagaaacacaatgggttcgtgtctacattgaaattaaataattaattacagctaattaataaacggcgaggttgacaatggtataactgtgaagagacagaatgagttcgtgactaaattggtattaaatgattaattagagctaattaacaggtggtggggctgagaatggcataactcaatgggggatgatgagctgatggcttaattggaattaataattaatcacaaccaattaacaggtgctgttaatggggttacaggggcatgacttcaggaagaagcacaaagtggccattaaataattaattgcacccaattaattcctgatggggattactgcagaggaactcagcgaaggattcaagcagaaatgacacagttaggttgctcaattgcgttaattaattaaaatcgctaattaggtgagcttcattaaggcctgatggggttcatcccaacgctgggatcaaagaactcagcgcagagtggataacgataattagaaggggacaaattaatgaggaaacacgactttgggcctagacggccattaaatcattaatcaacccgacgggcgcccaattaatcataaagttattaagcaattaagtcgaggctgacgagaaaccgcaacgagagccctccacgctcctttgagttggcgcaaacaacaaacaacgcgggctcgttaagcagcgacgacgccgagcgtaacgaagcaatccccgcgcccgacaattaacgcgctaacgaagccggagccctccttatgcaaattagcccctccagagggccataaaagcgccggctctccgacggaggagcaccagtcggctccgcaaccatgagcccactcgtctcctccctcctgctcctggccgccctgccaggtgagggcgctgtggggctctatggggctctatggggtctcagcggggctctgcgggctcaatgggggccaaagggggggtctgcgggctctatgggggggtcaacggggggtctcacggggggccggctccgcgaggccgtgtggcggcggctccgtcagcgctctctgcccttccccacagggctgcatgcggactagtccgccagatcccctaccgttcgtataatgtatgctatacgaagttatagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtcacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagccaagcttggtatggtaaaacttttaatttcgggtatatttgagtggaatgagttcttcaatcgtagttttgactaacttgccattcatttctattaacacaaaacaatctggtgcatagtctgaaatcaactccctacacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcttgttgagaaatgttaaatgttttcatatattcttcttcctccgatttctgcaaaaatgaatccaagcttgaattaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggctcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgaggatctgggctagccctgatcaataacttcgtataatgtatgctatacgaacggtaggggatctggcggcgattttggggcaaaatcgggcgattttgggtcagtcgaagggggcggtcggtccatcatttgcggccgggtgatttttggggccgaaaagtgggaatttggggcccaatttggggcccaatttggggcccaatttgggttttcgaggggggatttttttagggggagattttgggtccccggaaaatggtgattttgggtcaaaaatggggattttggggtcgttttgaggtggggttttttggggtagaaatggcggtctgaggggaaaatgtcgttttggggccattttgggccatttgaggggaaatttgggtcaaaaaatgacgattttgggtcgttttagggaggaaaatgaatttagggcgatttgagccaaatttgggtcaaaaatggcgattttgagtcaaaaaatgattattttgggtcattttagggaggaaaatgaatttagggagattggacggcaaatttgggtcaaaaaatggtgattttgggtcaaaaaatgattattttgggtcattttagggagaaaaatgaatttagggagatttgagggcaaatttgggtcaaaaaatggtgattttgggtcaaaaaatgattattttgggtcattttagggagaaaaatgaatttagggagatttgagggcaaatttgggtcgaaaaatggcgattttgggtgaaaaatggacaattttgggtcattttagggttaaaaatgaatttagggagattggacggcaaatttgggtcaaaaaatggtgatttggggtcaaaaaataattattttgggtcattttagggataaaaaatgaatttagggagatttgagggcaaatttgggtcgaaaaatggtgatttggggtgaaaaatggacaattttgggtcattttagggataaaaaatgaatttagggcgatttgagccaaatttgggtcaaaaatggcgattttgagtcaaaaaatgattattttgggtcattttagggttaaaaatgaatttagggagattggacggcaaatttgggtcaaaaaatggtgattttcggtcaaaaaatgattattttgggtcattttagggagaaaaatgaatttagggagattggacggcaaatttgggtcgaaaaatggtgattttgggtcaaaaatggacaatttggggtcattttagggataaaaatgaatttagggagatttgagagcaaatttgggtctaaaaatgtcaatttggggtcaaaaatggtgattttgggtcattttagggatataaatgaattttgggagatttgagggcaaatttgggtcgaaaaatggtgatttggggtcaatttagggaggaaaatgaatttaaggtgatttgacggcaaatttgggttgaaaaatgacaatttggggtcattttaaagataaaatgaatttagggcgatttgagggcaaatttgggtcgaagaatggtgatttgggtcaaaaaatgacaattttgggtcattttagggatataaatggacttagagcgatttgagggcaaatttggttgaaaaaatggacaatttgggtcattttagggatataaatgaatttagggagatttgatggcaaatttgggtcgaaaaatggtgatttgggtcaaaaatggtgattttggttgaaaaacagtcatttggggtcattttagggattaaaatgaatttcgggagatttgagggcaaatttgggtcaaaaaatggtgattttgggtcaattagtgataaaaaaatgaatttagggtgatttgagggcaaatttgggtcaaaaaatgacaatttggggtttttttagggaggagaatgaatttagggagttttgagggcaaatttgggtcaaaaaatagtgatttgggtcaaaagtggtgattttggttgaaaaacagtcattttgggtcattttagggagaaaaatgaatttcgggagatttgagggcaaatttgggtcaaaaaatgacaattttgggtcactttacgaattaaaatgaatttagggagatttgagggcaaatttgggtcaaaatatggtgattttgggtcaatttagggaggaaaatgaatttagggcgatttgaaggcaaatttgggtcgaaaatggtgattttgggtcaaaaaatgatgattttgggtcgttttagggagaaaatgaatttagggagatttgagggcaaatttgggtcaaaaaatggtgattttgggtcattgtagggataaaaatgaatttggggcgatttgagggcaaatttgggtggaaaatggacaattttgggtcattttagggagaaaaatgaatttagggagatttgagggcaaatttgggtcaaaaaatggtgatttggggtcattttcagaagaaaaaatgattattttccccactaaaaatgtatattttggggccaaatggtgaaaaatggtgatttttaatcaaacgtccccaaaattggggaaatttcatcgatttgacccaaaattgagttttttttccctgttaaaaatgtacattttggggtcaatcgctgaaatgttcccacttttcacttctttgcccccaaattttgctgtccggtgagaaattaattaattaatgatcctaaaggcagagtgctgtcaacattcctaattggttattacccaataacacacaccctcgaggccgc

SEQ ID NO: 14 - DNA, 27 bp, Artificial
  an artificially synthesized primer sequence
acgaccctggcaccaacagagacctgc

SEQ ID NO: 15 - DNA, 40 bp, Artificial
  an artificially synthesized primer sequence
actagttggttaaccgctgcctgcaccagggaacctggag

SEQ ID NO: 16 - DNA, 40 bp, Artificial
  an artificially synthesized primer sequence
actagtctcggatcctcttcccccatcgtgaaattgtgac

SEQ ID NO: 17 - DNA, 36 bp, Artificial
  an artificially synthesized primer sequence
agcgggtggagccatcgatgacccaatccacagtca

SEQ ID NO: 18 - DNA, 6879 bp, Artificial
  an artificially synthesized sequence
gagctcaagacaaggctgtttattactgctctggcaggaaagaagcactggccatggtcatagagagttccagcaacaggaaagtgagagcccaagctgctgaggtaccagggctcctcaggtgcctgctgcagcagcttggacacagtcgaggaacagcaattgtacctgtgtggtggatcaggctgtgctgcctgtgaacctattctagcacatctgtcacctctgtgccactcacagggataccacccctgagacccctaccccatcagcctctgtgtgggatatggtgttgggcccaagggctctgttgcacagggagatagaggcctggggaggagggaaagcattgaggtggtgttgataccagggatgtgagcccaagcaagagatcagcagagcaaggaggaagaattgcaggtgttggggctggggaaagccccagatggctggagctggtggggccactggagatctcctcctcccatcctgctccatgctggggcagctgctgcaggctgaccagggcctgcccgggcacgttgtgaaggtcaccaaggatggagacttcagagctatctgggacacgtgttttaacgtttgatcagccttgcagcaaaacagtgttttgctctattgaacagtgttcaacaaaaacgatcagtggcctggaacacatctcctacgaagaaaggttgcgagagctgggtctggagaagagaaggctctggggcaacctccctgtggcctgtcagtgcttaaaggaggctcaagagaaagatggagagagactttatacacggagctgtagcaatatgacaaggagaaatggtttaaaatggaaagagggtagatttaggttagctatcaagaagaaattcttcactcagaggtgatgaagcactgtagcaggttgcccagagaagtattggatgcttccttcctggcagggcccaaggccagactggatgggttctgggcagcctgttctggtgggagatgtccctgcctgtggcatggcttggaaatgggtagcctttgaggtccctctcagcccaaatcattctatgttcttgtggttccctgcatttcagcctttgcctgttgactctggtgccactgttggacaattctactcagctgcgtttcatccctgccccaaagcagtaatcccatggataagcaatgctctgaggccttcaccaccctcagggctcctgctgaccctagcggcctccctggcagcatgagccctttgttgtccgcactgctgctgctgagggtcctgccagccccccagggccttttctgcccaggcactgccacctgctaaaacccagcctgcccatggcagggcatcgcatgtctgagggatggaacggggcaagggcagtgctgcaggcagtggttcaagaaacgtttagatgttgtactgagggatgtggtttagtgggaaatactggtgataggtggatggttggactggaggatcttagaggtcttttccaaccttggtgattatgtgattctatgaagggagccatagcctgctcccatcatgccccgccagtccggctctcccgagcactttggttccgcagcatccaggcccgctctggggccggtgtgggcctgggtgctgccacgtgtccccgctgcaaggcctggccaaccccgctgtgccacaatgcaggggctgggctaggagtgggcagggaggtgtggggagaggcgtaggacgtggctgggagcgcagggagttatttgcataggggggcgtgctgcggaaggacgcgggtataaaagggcatcgaggtccccggcacagccccatcggcgtggggacacacagctgctgggattccgccatggcctgggctcctctcctcctggcggtgctcgcccacacctcaggtactcgttgcgcctggtcggggactgtgggcacggggctctgtcccattgctgcgcgggcagggctgtgcgtgcggggccgtcactgattgccgttttctcccctctctcctctccctctccaggttccctggtgcaggcagcggttaaccaactagtctcggatcccctaccgttcgtatagcatacattatacgaagttattgatcagggctagcccagatcctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaattcaagcttggattcatttttgcagaaatcggaggaagaagaatatatgaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaaagttttaccataccaagcttggctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctataacttcgtatagcatacattatacgaacggtaggggatcctcttcccccatcgtgaaattgtgacattttgtcgatttttggtgatttgggggtttttcttggacttggcggcaggctggggtctgccaccggcgcagggccgggcactcagcgcggcagcctgggctgagtcttgtccccaccgagccggagggctccggtgtgcgccatggaggacttagggttattttgtcaatggaaagttcttaaaatttgaccagaaaatgtgcccgaggtctgtctctgccacacaatttcagaaattgtgtctaggtcgatgagaagacagtttttgtctttgtcaggaaattagttgtgagttgttagtccttccctcttagtcctaaggactaagacctttgtccccggtctggtctctcactggggactcttggctccagtgccatggggagcccaagtgtcactgacacagtgtccttgggggtgaaattcagtttttcagctgtccaggtaagtgctgtcaaaaaatggaggttattctgctgaaaaagctgagaggaatattttgtcatttttttcggaaatatatatatatatatatatataaatatataaattatttatatattttatatatatatatatataaaatatatgtatttctctctttctttttatatatatatataaatatatatatatttctccctctttctttatatatatttatagggagagaatctatattttggccaatttggccaatttctctctctctctctttatatatatatatatatatatatgtaatttatatatatatatataaatttatatttatttatttatttatttatatagattatttttatatacatatatatatataaatttggccaatttagaagaaattggccaaaaaaacccaatctgaaccaaattacttagggcataagtccaaaaaaaaagtgcagttcaagaattcctcgctggaaagtttttaatgggggcagttcttccccattttctggtgtagatatgagtgaagatatagatatgtatgtagatatagatttggctatagtttgtttgatgaacagtctgaggaagagtaaatcttgccttcctcatggcaatttttaattctacttcttctgagacaagtctgaacatgccttgaggtagaaagagcccgaaaattgagtgctttttctttgaaattttaattaaaattaaaaaatagccttacagcaggaaagaaagggggtgatggcagctaatcggagttccaaagctgaaccacgttcacccaaacacgtggttgaaattttgtgtgtgtatatatataaatatatatatgtatacaaaagaaaatgcctatatataaaaaaattgttatgtatattatatagatttattatagatatgtacataagatataacacgtatacatatatatgtagtctgctgtatgctttgtataaatttttatagatatgatgtatattatatgcatgggttatatatctataatatattttatctgtagaaggaactacagtataaaatgcatatatgcgtatatatacacatatttatatatatgtgtatatatatgtatacgcacacgtataacatccatgaaaatagatatggatgggtggatgtgtttgttttacagaggtgcatgtgtgtctgtacatacacagccatacatacgcgtgtggccgctctgcctctctcttgcaggccagcccaaggtggcccccaccatcaccctcttcccaccgtcaaaggaggagctgaacgaagccaccaaggccaccctggtgtgcctgataaacgacttctaccccagcccagtgactgtggattgggtgatcgatggctccacccgctcctgaattc

SEQ ID NO: 19 - DNA, 39 bp, Artificial
  an artificially synthesized primer sequence
gaactagtgctgcatgcaccagggaacctggagagggag

SEQ ID NO: 20 - DNA, 6871 bp, Artificial
  an artificially synthesized sequence
gagctcaagacaaggctgtttattactgctctggcaggaaagaagcactggccatggtcatagagagttccagcaacaggaaagtgagagcccaagctgctgaggtaccagggctcctcaggtgcctgctgcagcagcttggacacagtcgaggaacagcaattgtacctgtgtggtggatcaggctgtgctgcctgtgaacctattctagcacatctgtcacctctgtgccactcacagggataccacccctgagacccctaccccatcagcctctgtgtgggatatggtgttgggcccaagggctctgttgcacagggagatagaggcctggggaggagggaaagcattgaggtggtgttgataccagggatgtgagcccaagcaagagatcagcagagcaaggaggaagaattgcaggtgttggggctggggaaagccccagatggctggagctggtggggccactggagatctcctcctcccatcctgctccatgctggggcagctgctgcaggctgaccagggcctgcccgggcacgttgtgaaggtcaccaaggatggagacttcagagctatctgggacacgtgttttaacgtttgatcagccttgcagcaaaacagtgttttgctctattgaacagtgttcaacaaaaacgatcagtggcctggaacacatctcctacgaagaaaggttgcgagagctgggtctggagaagagaaggctctggggcaacctccctgtggcctgtcagtgcttaaaggaggctcaagagaaagatggagagagactttatacacggagctgtagcaatatgacaaggagaaatggtttaaaatggaaagagggtagatttaggttagctatcaagaagaaattcttcactcagaggtgatgaagcactgtagcaggttgcccagagaagtattggatgcttccttcctggcagggcccaaggccagactggatgggttctgggcagcctgttctggtgggagatgtccctgcctgtggcatggcttggaaatgggtagcctttgaggtccctctcagcccaaatcattctatgttcttgtggttccctgcatttcagcctttgcctgttgactctggtgccactgttggacaattctactcagctgcgtttcatccctgccccaaagcagtaatcccatggataagcaatgctctgaggccttcaccaccctcagggctcctgctgaccctagcggcctccctggcagcatgagccctttgttgtccgcactgctgctgctgagggtcctgccagccccccagggccttttctgcccaggcactgccacctgctaaaacccagcctgcccatggcagggcatcgcatgtctgagggatggaacggggcaagggcagtgctgcaggcagtggttcaagaaacgtttagatgttgtactgagggatgtggtttagtgggaaatactggtgataggtggatggttggactggaggatcttagaggtcttttccaaccttggtgattatgtgattctatgaagggagccatagcctgctcccatcatgccccgccagtccggctctcccgagcactttggttccgcagcatccaggcccgctctggggccggtgtgggcctgggtgctgccacgtgtccccgctgcaaggcctggccaaccccgctgtgccacaatgcaggggctgggctaggagtgggcagggaggtgtggggagaggcgtaggacgtggctgggagcgcagggagttatttgcataggggggcgtgctgcggaaggacgcgggtataaaagggcatcgaggtccccggcacagccccatcggcgtggggacacacagctgctgggattccgccatggcctgggctcctctcctcctggcggtgctcgcccacacctcaggtactcgttgcgcctggtcggggactgtgggcacggggctctgtcccattgctgcgcgggcagggctgtgcgtgcggggccgtcactgattgccgttttctcccctctctcctctccctctccaggttccctggtgcatgcagcactagtctcggatcccctaccgttcgtatagcatacattatacgaagttattgatcagggctagcccagatcctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaattcaagcttggattcatttttgcagaaatcggaggaagaagaatatatgaaaacatttaacatttctcaacaagatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaaagttttaccataccaagcttggctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctataacttcgtatagcatacattatacgaacggtaggggatcctcttcccccatcgtgaaattgtgacattttgtcgatttttggtgatttgggggtttttcttggacttggcggcaggctggggtctgccaccggcgcagggccgggcactcagcgcggcagcctgggctgagtcttgtccccaccgagccggagggctccggtgtgcgccatggaggacttagggttattttgtcaatggaaagttcttaaaatttgaccagaaaatgtgcccgaggtctgtctctgccacacaatttcagaaattgtgtctaggtcgatgagaagacagtttttgtctttgtcaggaaattagttgtgagttgttagtccttccctcttagtcctaaggactaagacctttgtccccggtctggtctctcactggggactcttggctccagtgccatggggagcccaagtgtcactgacacagtgtccttgggggtgaaattcagtttttcagctgtccaggtaagtgctgtcaaaaaatggaggttattctgctgaaaaagctgagaggaatattttgtcatttttttcggaaatatatatatatatatatatataaatatataaattatttatatattttatatatatatatatataaaatatatgtatttctctctttctttttatatatatatataaatatatatatatttctccctctttctttatatatatttatagggagagaatctatattttggccaatttggccaatttctctctctctctctttatatatatatatatatatatatgtaatttatatatatatatataaatttatatttatttatttatttatttatatagattatttttatatacatatatatatataaatttggccaatttagaagaaattggccaaaaaaacccaatctgaaccaaattacttagggcataagtccaaaaaaaaagtgcagttcaagaattcctcgctggaaagtttttaatgggggcagttcttccccattttctggtgtagatatgagtgaagatatagatatgtatgtagatatagatttggctatagtttgtttgatgaacagtctgaggaagagtaaatcttgccttcctcatggcaatttttaattctacttcttctgagacaagtctgaacatgccttgaggtagaaagagcccgaaaattgagtgctttttctttgaaattttaattaaaattaaaaaatagccttacagcaggaaagaaagggggtgatggcagctaatcggagttccaaagctgaaccacgttcacccaaacacgtggttgaaattttgtgtgtgtatatatataaatatatatatgtatacaaaagaaaatgcctatatataaaaaaattgttatgtatattatatagatttattatagatatgtacataagatataacacgtatacatatatatgtagtctgctgtatgctttgtataaatttttatagatatgatgtatattatatgcatgggttatatatctataatatattttatctgtagaaggaactacagtataaaatgcatatatgcgtatatatacacatatttatatatatgtgtatatatatgtatacgcacacgtataacatccatgaaaatagatatggatgggtggatgtgtttgttttacagaggtgcatgtgtgtctgtacatacacagccatacatacgcgtgtggccgctctgcctctctcttgcaggccagcccaaggtggcccccaccatcaccctcttcccaccgtcaaaggaggagctgaacgaagccaccaaggccaccctggtgtgcctgataaacgacttctaccccagcccagtgactgtggattgggtgatcgatggctccacccgctcctgaattct

SEQ ID NO: 21 - DNA, 24 bp, Artificial
  an artificially synthesized sequence
gaggttcagctgcagcagtctggg

SEQ ID NO: 22 - DNA, 29 bp, Artificial
  an artificially synthesized sequence
actagtactcacctgaggagacggtgact

SEQ ID NO: 23 - DNA, 22 bp, Artificial
  an artificially synthesized sequence
ggcggctccgtcagcgctctct

SEQ ID NO: 24 - DNA, 26 bp, Artificial
  an artificially synthesized sequence
ttcaccgccttgggttgcaacggtgg

SEQ ID NO: 25 - DNA, 22 bp, Artificial
  an artificially synthesized sequence
ggcggctccgtcagcgctctct

SEQ ID NO: 26 - DNA, 20 bp, Artificial
  an artificially synthesized sequence
gccgcaaatgatggaccgac

SEQ ID NO: 27 - DNA, 30 bp, Artificial
  an artificially synthesized sequence
actcaggaatctgcactcaccacatcacct

SEQ ID NO: 28 - DNA, 38 bp, Artificial
  an artificially synthesized sequence
gttctagacactcacctaggacagtcagtttggttcct

SEQ ID NO: 29 - DNA, 28 bp, Artificial
  an artificially synthesized sequence
tgcctggggtaagggtagtactctgtgc

SEQ ID NO: 30 - DNA, 23 bp, Artificial
  an artificially synthesized sequence
aacggtaggggatccgagactag

SEQ ID NO: 31 - DNA, 41 bp, Artificial
  an artificially synthesized sequence
gagaaaggtagaagaccccaaggactttccttcagaattgc

SEQ ID NO: 32 - DNA, 30 bp, Artificial
  an artificially synthesized sequence
gcagagtcagcactagttcagtgtcgtgtt

SEQ ID NO: 33 - DNA, 31 bp, Artificial
  an artificially synthesized sequence
caggagctcgcggggccgtcactgattgccg

SEQ ID NO: 34 - DNA, 34 bp, Artificial
  an artificially synthesized sequence
gcgcaagcttccccagcctgccgccaagtccaag

SEQ ID NO: 35 - DNA, 30 bp, Artificial
  an artificially synthesized sequence
ctgactgaccgcgttactcccacagccagc

SEQ ID NO: 36 - DNA, 30 bp, Artificial
  an artificially synthesized sequence
ttcatgaggtagtccgtcaggtcacggcca

SEQ ID NO: 37 - DNA, 34 bp, Artificial
  an artificially synthesized sequence
taccgttcgtataatgtatgctatacgaagttat

SEQ ID NO: 38 - DNA, 34 bp, Artificial
  an artificially synthesized sequence
ataacttcgtataatgtatgctatacgaacggta

SEQ ID NO: 39 - DNA, 1953 bp, Gallus gallus
gtgatctcgggttgggtcgctcgcgtccgccgcactaagcggttcggagacctgccgggcagacgccggcgtagccacagataatttcagcgagcttaagctctcgtcagaatgccgcgttgaataggtgccgcctagcgataacgctcgtcttgccctccgccctccgcctgccctcagaccccatggcagccgccgtgccccgcgctccacggtgccgtatcaggacatggactgggaccagcttgacctgaacccgaaggtgattgctgcccttaagaaagctgacataaaatcgataaaggaggtgttaaacctttctggagcagacttgcaaaggctgatgaagctgtcgagtgcagatgtggagtgtctgctgaaaacggtctctcgcgctctgaggaaaaactgcatgcttacagcacttcagctctaccaagataaggattgtttctcctcccagtgccagaagctgagtctgggatgttcagtgcttgatgacttgttgaaaggtggcattcctttggtgggaatcacagaacttgctggggaaagttctgctgggaagactcagattggtttgcagctgtgcctttgtgtgcagtatccttacagatacggtggattagagtcaggagctgtctacatttgtacagaagatgcattcccaagtaaacgtttgcagcagctgatagctcaacagcataagctgcgtgctgatgtgccacctgaaatcatacaaaagatcaggtttgggaacagtatctttgttgagcatgcagcagacctggacaccttccacaactgcattacgaagaggctgtccctgctgctcacaagaggcatggtgcggttggtggtcatcgattccattgctgctttgtttcgatgtgaatttggggcttcagattctgttctgaaggctcgctacttgcagacgtttggagcacagctccatggcttaagcaccagattcaggactccaataatgtgcattaatcaggtgaccgatgctgtgagcgagtcagaagctgcacagtgtagctacagcactgctgatagcagagtctctccagcactaggaataacttgggcaaatcagctgctcatgagactgatggtcagcagagtgccacaacctgaaccgtcacctggagctgtgtcacaccaccctgcaagcatgaggactttaagagtagtgtttgctcctcatctacctccatccttctgctgctttacagtaaaattggaaggtgtgaaaggaatgaagtaagttctttctgcagtatagcatcctggtccaaaagtgcattgaacgtactttgcttcaggaaggagcattagtaaaatgaaattcttcctagagcagaggaagggatgctgaaatacatagaaggtgatatgacaacaacattaagcatttttttttttatgttgtgcaaagatgctctttttgtatctggatagaagttcaccagcctctccttgtgagataagtgttttggagaataatccttactgcatcccactaatcccactgatgagtggcacctgcattcatccacacagcccctgctttcctcagaggcttttatcagagctctacatgttttttcttcccatctttgtgctatttattgcatctttttctgctggcctcaagactaactgctctgggtgcttttttgtaatcgtcgctgcattcggggtgaaggacagctacatgaacttattcatccacactttccaaaggccttaaaacttactgtgtggtgggtgggtgttgttatcagggggtgacactcacttgtatagctgttaagctttcacacatttccctctcccatctcaccccttctccccaggttgcccagtctgtatttgtcagtgatgacagtaatctttttaattatttttttaaataaaatccaatgacgaaaaaaaaaaaaaaaa

SEQ ID NO: 40 - DNA, 957 bp, Mus musculus
cgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaacgcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatc

SEQ ID NO: 41 - protein, 319 aa, Mus musculus
RNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKRMEHLYNMKCKNVVPLYDLLLEMLDAHRLHAPASRMGVPPEEPSQTQLATTSSTSAHSLQTYYIPPEAEGFPNTI

SEQ ID NO: 42 - DNA, 13 bp, Artificial
  an artificially synthesized sequence
ggtgagtactagt

SEQ ID NO: 43 - DNA, 10 bp, Artificial
  an artificially synthesized sequence
cagatctggc

SEQ ID NO: 44 - DNA, 1395 bp, Gallus gallus
ggatccctaattaatgttggcgcagaagggattcatatcaattaattccctgtcctcaattaatgcataattgggagtgcgatggaatcattcaataagtaatgaggacttggtgtcaaaatgccaattaagtaattaatgacacccaattaatacacaatggggattacagtggaataactcagtgaagttcatatctaaattgcaattgaataattaattacagctaattaatggatgggagactggcagtggtataacttaatgaaggaacacagtgaattcctgtctgaattgaaattaattgatttattagggctaattaatgaatggtggggttaaaaatagcattactcagcgaaggaacatgagttcatggctaaattgagattgaattattaattacaactaattgattaatgactgatgggagtaagaatggaataactcagtgaaggaacacaatggattcatggcctaattgaaattaaatggttaattagagctaattaatgaatgatggggggtggcaatggaatgatacaagaaagaaacacaatgggttcgtgtctacattgaaattaaataattaattacagctaattaataaacggcgaggttgacaatggtataactgtgaagagacagaatgagttcgtgactaaattggtattaaatgattaattagagctaattaacaggtggtggggctgagaatggcataactcaatgggggatgatgagctgatggcttaattggaattaataattaatcacaaccaattaacaggtgctgttaatggggttacaggggcatgacttcaggaagaagcacaaagtggccattaaataattaattgcacccaattaattcctgatggggattactgcagaggaactcagcgaaggattcaagcagaaatgacacagttaggttgctcaattgcgttaattaattaaaatcgctaattaggtgagcttcattaaggcctgatggggttcatcccaacgctgggatcaaagaactcagcgcagagtggataacgataattagaaggggacaaattaatgaggaaacacgactttgggcctagacggccattaaatcattaatcaacccgacgggcgcccaattaatcataaagttattaagcaattaagtcgaggctgacgagaaaccgcaacgagagccctccacgctcctttgagttggcgcaaacaacaaacaacgcgggctcgttaagcagcgacgacgccgagcgtaacgaagcaatccccgcgcccgacaattaacgcgctaacgaagccggagccctccttatgcaaattagcccctccagagggccataaaagcgccggctctccgacggaggagcaccagtcggctccgcaacc

SEQ ID NO: 45 - DNA, 2680 bp, Gallus gallus
gcggcgattttggggcaaaatcgggcgattttgggtcagtcgaagggggcggtcggtccatcatttgcggccgggtgatttttggggccgaaaagtgggaatttggggcccaatttggggcccaatttggggcccaatttgggttttcgaggggggatttttttagggggagattttgggtccccggaaaatggtgattttgggtcaaaaatggggattttggggtcgttttgaggtggggttttttggggtagaaatggcggtctgaggggaaaatgtcgttttggggccattttgggccatttgaggggaaatttgggtcaaaaaatgacgattttgggtcgttttagggaggaaaatgaatttagggcgatttgagccaaatttgggtcaaaaatggcgattttgagtcaaaaaatgattattttgggtcattttagggaggaaaatgaatttagggagattggacggcaaatttgggtcaaaaaatggtgattttgggtcaaaaaatgattattttgggtcattttagggagaaaaatgaatttagggagatttgagggcaaatttgggtcaaaaaatggtgattttgggtcaaaaaatgattattttgggtcattttagggagaaaaatgaatttagggagatttgagggcaaatttgggtcgaaaaatggcgattttgggtgaaaaatggacaattttgggtcattttagggttaaaaatgaatttagggagattggacggcaaatttgggtcaaaaaatggtgatttggggtcaaaaaataattattttgggtcattttagggataaaaaatgaatttagggagatttgagggcaaatttgggtcgaaaaatggtgatttggggtgaaaaatggacaattttgggtcattttagggataaaaaatgaatttagggcgatttgagccaaatttgggtcaaaaatggcgattttgagtcaaaaaatgattattttgggtcattttagggttaaaaatgaatttagggagattggacggcaaatttgggtcaaaaaatggtgattttcggtcaaaaaatgattattttgggtcattttagggagaaaaatgaatttagggagattggacggcaaatttgggtcgaaaaatggtgattttgggtcaaaaatggacaatttggggtcattttagggataaaaatgaatttagggagatttgagagcaaatttgggtctaaaaatgtcaatttggggtcaaaaatggtgattttgggtcattttagggatataaatgaattttgggagatttgagggcaaatttgggtcgaaaaatggtgatttggggtcaatttagggaggaaaatgaatttaaggtgatttgacggcaaatttgggttgaaaaatgacaatttggggtcattttaaagataaaatgaatttagggcgatttgagggcaaatttgggtcgaagaatggtgatttgggtcaaaaaatgacaattttgggtcattttagggatataaatggacttagagcgatttgagggcaaatttggttgaaaaaatggacaatttgggtcattttagggatataaatgaatttagggagatttgatggcaaatttgggtcgaaaaatggtgatttgggtcaaaaatggtgattttggttgaaaaacagtcatttggggtcattttagggattaaaatgaatttcgggagatttgagggcaaatttgggtcaaaaaatggtgattttgggtcaattagtgataaaaaaatgaatttagggtgatttgagggcaaatttgggtcaaaaaatgacaatttggggtttttttagggaggagaatgaatttagggagttttgagggcaaatttgggtcaaaaaatagtgatttgggtcaaaagtggtgattttggttgaaaaacagtcattttgggtcattttagggagaaaaatgaatttcgggagatttgagggcaaatttgggtcaaaaaatgacaattttgggtcactttacgaattaaaatgaatttagggagatttgagggcaaatttgggtcaaaatatggtgattttgggtcaatttagggaggaaaatgaatttagggcgatttgaaggcaaatttgggtcgaaaatggtgattttgggtcaaaaaatgatgattttgggtcgttttagggagaaaatgaatttagggagatttgagggcaaatttgggtcaaaaaatggtgattttgggtcattgtagggataaaaatgaatttggggcgatttgagggcaaatttgggtggaaaatggacaattttgggtcattttagggagaaaaatgaatttagggagatttgagggcaaatttgggtcaaaaaatggtgatttggggtcattttcagaagaaaaaatgattattttccccactaaaaatgtatattttggggccaaatggtgaaaaatggtgatttttaatcaaacgtccccaaaattggggaaatttcatcgatttgacccaaaattgagttttttttccctgttaaaaatgtacattttggggtcaatcgctgaaatgttcccacttttcacttctttgcccccaaattttgctgtccggtgagaaattaattaattaatgatcctaaaggcagagtgctgtcaacattcctaattggttattacccaataacacacaccctcgag

SEQ ID NO: 46 - DNA, 1868 bp, Gallus gallus
gagctcaagacaaggctgtttattactgctctggcaggaaagaagcactggccatggtcatagagagttccagcaacaggaaagtgagagcccaagctgctgaggtaccagggctcctcaggtgcctgctgcagcagcttggacacagtcgaggaacagcaattgtacctgtgtggtggatcaggctgtgctgcctgtgaacctattctagcacatctgtcacctctgtgccactcacagggataccacccctgagacccctaccccatcagcctctgtgtgggatatggtgttgggcccaagggctctgttgcacagggagatagaggcctggggaggagggaaagcattgaggtggtgttgataccagggatgtgagcccaagcaagagatcagcagagcaaggaggaagaattgcaggtgttggggctggggaaagccccagatggctggagctggtggggccactggagatctcctcctcccatcctgctccatgctggggcagctgctgcaggctgaccagggcctgcccgggcacgttgtgaaggtcaccaaggatggagacttcagagctatctgggacacgtgttttaacgtttgatcagccttgcagcaaaacagtgttttgctctattgaacagtgttcaacaaaaacgatcagtggcctggaacacatctcctacgaagaaaggttgcgagagctgggtctggagaagagaaggctctggggcaacctccctgtggcctgtcagtgcttaaaggaggctcaagagaaagatggagagagactttatacacggagctgtagcaatatgacaaggagaaatggtttaaaatggaaagagggtagatttaggttagctatcaagaagaaattcttcactcagaggtgatgaagcactgtagcaggttgcccagagaagtattggatgcttccttcctggcagggcccaaggccagactggatgggttctgggcagcctgttctggtgggagatgtccctgcctgtggcatggcttggaaatgggtagcctttgaggtccctctcagcccaaatcattctatgttcttgtggttccctgcatttcagcctttgcctgttgactctggtgccactgttggacaattctactcagctgcgtttcatccctgccccaaagcagtaatcccatggataagcaatgctctgaggccttcaccaccctcagggctcctgctgaccctagcggcctccctggcagcatgagccctttgttgtccgcactgctgctgctgagggtcctgccagccccccagggccttttctgcccaggcactgccacctgctaaaacccagcctgcccatggcagggcatcgcatgtctgagggatggaacggggcaagggcagtgctgcaggcagtggttcaagaaacgtttagatgttgtactgagggatgtggtttagtgggaaatactggtgataggtggatggttggactggaggatcttagaggtcttttccaaccttggtgattatgtgattctatgaagggagccatagcctgctcccatcatgccccgccagtccggctctcccgagcactttggttccgcagcatccaggcccgctctggggccggtgtgggcctgggtgctgccacgtgtccccgctgcaaggcctggccaaccccgctgtgccacaatgcaggggctgggctaggagtgggcagggaggtgtggggagaggcgtaggacgtggctgggagcgcagggagttatttgcataggggggcgtgctgcggaaggacgcgggtataaaagggcatcgaggtccccggcacagccccatcggcgtggggacacacagctgctgggattccgcc

SEQ ID NO: 47 - DNA, 1860 bp, Gallus gallus
tcttcccccatcgtgaaattgtgacattttgtcgatttttggtgatttgggggtttttcttggacttggcggcaggctggggtctgccaccggcgcagggccgggcactcagcgcggcagcctgggctgagtcttgtccccaccgagccggagggctccggtgtgcgccatggaggacttagggttattttgtcaatggaaagttcttaaaatttgaccagaaaatgtgcccgaggtctgtctctgccacacaatttcagaaattgtgtctaggtcgatgagaagacagtttttgtctttgtcaggaaattagttgtgagttgttagtccttccctcttagtcctaaggactaagacctttgtccccggtctggtctctcactggggactcttggctccagtgccatggggagcccaagtgtcactgacacagtgtccttgggggtgaaattcagtttttcagctgtccaggtaagtgctgtcaaaaaatggaggttattctgctgaaaaagctgagaggaatattttgtcatttttttcggaaatatatatatatatatatatataaatatataaattatttatatattttatatatatatatatataaaatatatgtatttctctctttctttttatatatatatataaatatatatatatttctccctctttctttatatatatttatagggagagaatctatattttggccaatttggccaatttctctctctctctctttatatatatatatatatatatatgtaatttatatatatatatataaatttatatttatttatttatttatttatatagattatttttatatacatatatatatataaatttggccaatttagaagaaattggccaaaaaaacccaatctgaaccaaattacttagggcataagtccaaaaaaaaagtgcagttcaagaattcctcgctggaaagtttttaatgggggcagttcttccccattttctggtgtagatatgagtgaagatatagatatgtatgtagatatagatttggctatagtttgtttgatgaacagtctgaggaagagtaaatcttgccttcctcatggcaatttttaattctacttcttctgagacaagtctgaacatgccttgaggtagaaagagcccgaaaattgagtgctttttctttgaaattttaattaaaattaaaaaatagccttacagcaggaaagaaagggggtgatggcagctaatcggagttccaaagctgaaccacgttcacccaaacacgtggttgaaattttgtgtgtgtatatatataaatatatatatgtatacaaaagaaaatgcctatatataaaaaaattgttatgtatattatatagatttattatagatatgtacataagatataacacgtatacatatatatgtagtctgctgtatgctttgtataaatttttatagatatgatgtatattatatgcatgggttatatatctataatatattttatctgtagaaggaactacagtataaaatgcatatatgcgtatatatacacatatttatatatatgtgtatatatatgtatacgcacacgtataacatccatgaaaatagatatggatgggtggatgtgtttgttttacagaggtgcatgtgtgtctgtacatacacagccatacatacgcgtgtggccgctctgcctctctcttgcaggccagcccaaggtggcccccaccatcaccctcttcccaccgtcaaaggaggagctgaacgaagccaccaaggccaccctggtgtgcctgataaacgacttctaccccagcccagtgactgtggattgggtgatcgatggctccacccgct

SEQ ID NO: 48 - DNA, 33 bp, Artificial
  an artificially synthesized sequence
actcagccgtcctcggtgtcagcaaacccggga

SEQ ID NO: 49 - DNA, 35 bp, Artificial
  an artificially synthesized sequence
cgagactagttcagcgactcacctaggacggtcag

SEQ ID NO: 50 - DNA, 1112 bp, Gallus gallus
ctgacatggacagcctcttgatgaagaggaagctcttcctctacaatttcaagaacctgcgctgggccaaaggccgtcgtgaaacctacctctgttatgttgtgaagcgccgtgacagtgctacatcatgctccctggactttggatacctgcgtaacaagatgggttgccatgtggaggttctcttcctacgctacatctcagcttgggacctggacccaggccgctgctaccgcatcacatggttcacctcctggagcccctgttatgactgtgcccgacatgtggctgacttccttcgtgcctacccaaacttgaccctccgcattttcactgcccgcctctacttctgtgaagatcgcaaggctgagcctgaggggctgagacgcctgcaccgggctggggcccaaatcgccatcatgactttcaaagatttcttctactgctggaacacgtttgtggagaacagggaaaagacattcaaagcctgggaagggctgcatgaaaactctgtccatctgtccaggaaactccgacggatccttctgccactgtatgaagtagatgatttacgagatgcctttaaaactctgggactttgaagaggaagtcatcagcaataagaagacttcacaagcatttgttctttgatattcaactctctccctcactgtgtatctgtgtcttcttccttctttacatctctctttcttccagacatgtctaaatataagacaattcgtattttttctgtcctaacagccaaattgaaaagctctcgtgagagtactgaactgagtcctggacagctcagtttgaacctcatttgaaaccagcaaatcttctaagtggtttgtgtgcagatctgggccctcttgcacagaatgaagggagcttgaatggcggtgggtccagattttgtttgctatgacaggttgaaactagacagtaaaagcctaaaatgaaagcaaaagattttctgtccaacattttcaagtgtgagctgcagaaataggaaatccactcaatacctactcactacattgagagccatggcagctccacatcagtgctgatcactgtatggacctctcacccagccaagaaaggag

SEQ ID NO: 51 - DNA, 1053 bp, Bacteriophage P1
atgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 52 - DNA, 4319 bp, Mus musculus
acaaggctccctgctcagcagccttgtgatgccaggagaggccaatgccccctcctctgccattgtctagcttaagaaggcagtctctgggcgacattcttctcaagcaggtggcccacgcgctgctgagccctctgcgtgcgcggggagccagtctgtaactcgccggctgccacttaccatgaccatgacccttcacaccaaagcctcgggaatggccttgctgcaccagatccaagggaacgagctggagcccctcaaccgcccgcagctcaagatgcccatggagagggccctgggcgaggtatacgtggacaacagcaagcccactgtgttcaactaccccgagggcgccgcctacgagttcaacgccgccgccgccgccgccgccgccgcctcggcgccggtctacggccagtcgggcatcgcctacggccccgggtcggaggcggccgccttcagtgccaacagcctgggggctttcccccagctcaacagcgtgtcgcctagcccgctgatgctgctgcacccgccgccgcagctgtctcctttcctgcacccgcacggccagcaggtgccctactacctggagaacgagcccagcgcctacgccgtgcgcgacaccggccctcccgccttctacaggtctaattctgacaatcgacgccagaatggccgagagagactgtccagcagtaacgagaaaggaaacatgatcatggagtctgccaaggagactcgctactgtgccgtgtgcaatgactatgcctctggctaccattatggggtctggtcctgcgaaggctgcaaggctttctttaagagaagcattcaaggacacaatgactacatgtgtccagctacaaaccaatgcaccattgacaagaaccggaggaagagttgccaggcctgtcggctgcgcaagtgttacgaagtgggcatgatgaaaggcggcatacggaaagaccgccgaggagggagaatgttgaagcacaagcgtcagagagatgacttggaaggccgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgctggctacgtcaagtcggttccgcatgatgaacctgcagggagaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaaggcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctgagagctcccaggctccccgaaaggttctgagaatccctgcaacattttacccatgtcatgtatgactagcagaattctgtctcctgcgtacactgtggcatgcatccaccaccagtggctttctagtatgagtggccatttatttgcttgctcagttcttagtggcatgtcttctgttgggaacaggcaaaagggattccagggctaaatcttggtaacagctctctttctccctttgctacgtcactaactatgaggattcctatagtcttcagtcttcatgaatgaattgagataactgcattgaagctacttgttgagacccaagcctagagagtaaacatttcacctctgataagcacttcataatggctccaagaggaagcctcagcaaagacttcaaagtggctcctttaattgaagtcgttgacaaagcgatctcatggcttcctaatagcacccgcttgcattcctatagcaacatgtgactttattaaagggaaatcttgagcccctataggaatatgtgatgcacacagaaaaggaagatgcccccagttgtcaagaccatgtcaactcaatacagtcaaccttttcacttcttaaagtgtcagctgccgagtattatatacatggtgggttctagacaattcttgcttcccatgacttgtgggaaacagaaagtctatttcagttaataccctggaatgaattagtctccttcatgagggctgtagggtgtgttgattttaatttgatttcattttataatagtcttctgcccctggggcttgcagtaaaggcagcttgaggacctgttctggtaaactggacattcttcagcacaggatgctagccttgtctcttccctgatgtcaattgccctaaattgttaccatgtagattgatcaagggcagtcacaatgaacctgcaagctggtgacaattttttggtttgtttagttttattcttgggggcagggaacaagtaatgtatgggcaatgactgtgactcggaggagtctaagaaggaaagtaagggagaaatgtggagaatgacctggccagggcagcagcttcccagaggtgcacctcacagatcccaccatgcacagtgaggctgctctgggatccatgggctgacttcacttacatttcaggtatgagcctgattggaagaggtgacatcagatacctgtgttcctgtagatgctccaggaacttggggaaaggcatgggggcaagatcttggagcactcatgactcaagctgttgagaagttgctcagactagactgtgcttgattattctgaactcatccaaaaaccagctagtgagatcttggggagaaaaatttcttcctcaacacagttcatgatcttaaattttgtaaactagaaatgatgaatgaccagaattgggtcatgagagtcctttgaacaaggggataaaaaggaagacttactctatccacagccacccaagacctctggggtcttggtctctctagtacaactaaggattacttccttttccaaaccaaaatgaaagttcagtttccatcccggacttcatgatctataatttgaactccattgagatatgatgggttagccaaaacctaggtgaatggctaggcttctcttggcctgtactttttggaatggtgggttccattaatttctgctctcccagttaactgactatcaaagatgtggaaactgggagagccaatttttcttcttattttttttaaatttctcatacttgttcatcaaattcaaggacaattttatgtatatgctttaagaacatgtttaagaacctaactcttttgtttgtgtttaagaagcaccttatatagtataatatatatttttgttgaagtttaattgcttgtttattggacaattgaatgtaatagttatgttctgagttttaatttgattgggtttaacactgcaaaagccaaggggaaaatatttagtttgttttttttttttttttttgtagacgtttcaaactaccgtgtcatgtatgcagttattaatgcctaaagccttgtgattttcatttaaatgaagatcacatttcgtatcaacttttgtatccacagtagacaaaatagaaggaatccacatgcctattgctgggtgttgaatgacagacaatcttatgtagcagagattatgcctgggaaggaaaattattcagggcagctaattttgcttttaccaaaatatctgtagtaatatttttggacagtagctaatgggtcagtgggttctttttaatgtttatactgagatttttcttttacaaaaataaaaacaaaaatt

SEQ ID NO: 53 - protein, 599 aa, Mus musculus
MTMTLHTKASGMALLHQIQGNELEPLNRPQLKMPMERALGEVYVDNSKPTVFNYPEGAAYEFNAAAAAAAAASAPVYGQSGIAYGPGSEAAAFSANSLGAFPQLNSVSPSPLMLLHPPPQLSPFLHPHGQQVPYYLENEPSAYAVRDTGPPAFYRSNSDNRRQNGRERLSSSNEKGNMIMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDLEGRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI